(19) United States
(12) Patent Application Publication
(10) Pub. No.: US 2005/0245543 A1
(43) Pub. Date: Nov. 3, 2005

(54) Histamine-3 receptor antagonists

(75) Inventors: Harry R. Howard, Bristol, CT (US); Bishop Wlodecki, Preston, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/107,457

(22) Filed: Apr. 15, 2005

Related U.S. Application Data
(60) Provisional application No. 60/566,849, filed on Apr. 30, 2004.

(51) Int. Cl. A61K 31/506; A61K 31/454; A61K 31/4439; A61K 31/40; C07D 43/02; C07D 41/02

(52) U.S. Cl. 514/256; 514/343; 514/326; 514/357; 514/408; 544/333; 546/207; 546/276.4; 546/259; 548/577

(57) ABSTRACT

This invention is directed to a compound of the formula I 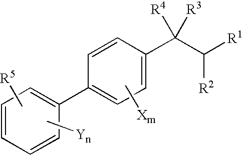 as defined herein, or a pharmaceutically acceptable salt thereof; a pharmaceutical composition containing a compound of formula I, a method of treatment of a disorder or condition that may be treated by antagonizing histamine H3 receptors, the method comprising administering to a mammal in need of such treatment a compound of formula I as described above, and a method of treatment of a disorder or condition selected from the group consisting of depression, mood disorders, schizophrenia, anxiety disorders, Alzheimer's disease, attention-deficit disorder (ADD), attention-deficit hyperactivity disorder (ADHD), psychotic disorders, sleep disorders, obesity, dizziness, epilepsy, motion sickness, respiratory diseases, allergy, allergy-induced airway responses, allergic rhinitis, nasal congestion, allergic congestion, congestion, hypotension, cardiovascular disease, diseases of the GI tract, hyper and hypo motility and acidic secretion of the gastro-intestinal tract, the method comprising administering to a mammal in need of such treatment a compound of formula I as described above.

### BACKGROUND OF THE INVENTION

This invention is directed to compounds of formula I described herein, to a pharmaceutical composition comprising such compounds, and to methods of treatment of disorders or conditions that may be treated by antagonizing histamine-3 (H3) receptors using such compounds. The histamine-3 (H3) receptor antagonists of the invention are useful for treating anxiety disorders, including, for example, generalized anxiety disorder, panic disorder, PTSD, and social anxiety disorder; mood adjustment disorders, including depressed mood, mixed anxiety and depressed mood, disturbance of conduct, and mixed disturbance of conduct and depressed mood; age-associated learning and mental disorders, including Alzheimer's disease; attention adjustment disorders, such as attention-deficit disorders, or other cognitive disorders due to general medical conditions; attention-deficit hyperactivity disorder; psychotic disorders including schizoaffective disorders and schizophrenia; sleep disorders, including narcolepsy and enuresis; obesity; dizziness, epilepsy, and motion sickness. The H3 receptor antagonists of the invention are also useful for treating, for example, allergy, allergy-induced airway (e.g., upper airway) responses, congestion (e.g., nasal congestion), hypotension, cardiovascular disease, diseases of the GI tract, hyper and hypo motility and acidic secretion of the gastrointestinal tract, sleeping disorders (e.g., hypersomnia, somnolence, and narcolepsy), disturbances of the central nervous system, attention deficit hyperactivity disorder (ADHD), hypo and hyperactivity of the central nervous system (for example, agitation and depression), and other CNS disorders (such as schizophrenia and migraine).

Histamine is a well-known mediator in hypersensitive reactions (e.g. allergies, hay fever, and asthma) that are commonly treated with antagonists of histamine or “antihistamines.” It has also been established that histamine receptors exist in at least two distinct types, referred to as H1 and H2 receptors.

A third histamine receptor (H3 receptor) is believed to play a role in neurotransmission in the central nervous system, where the H3 receptor is thought to be disposed presynaptically on histaminergic nerve endings (Nature, 302, S32-837 (1983)). The existence of the H3 receptor has been confirmed by the development of selective H3 receptor agonists and antagonists (Nature, 327, 117-123 (1987)) and has subsequently been shown to regulate the release of the neurotransmitters in both the central nervous system and peripheral organs, particularly the lungs, cardiovascular system and gastrointestinal tract.

A number of diseases or conditions may be treated with histamine-3 receptor ligands wherein the H3 ligand may be an antagonist, agonist or partial agonist, see: (Imamura et al., Circ. Res., (1996) 78, 475-481); (Imamura et. al., Circ. Res., (1996) 78, 863-869); (Lin et al., Brain Res. (1990) 523, 325-330); (Monti et al., Neuropsychopharmacology (1996) 15, 31 35); (Sakai, et al., Life Sci. (1991) 48, 2397-2404); (Mazurkiewiez-Kwilecki and Nsonwah, Can. J. Physiol. Pharmacol. (1989) 67, 75-78); (Panula, P. et al.,

Neuroscience (1998) 44, 465-481); (Wada et al., Trends in Neuroscience (1991) 14, 415); (Monti et al., Eur. J. Pharmacol. (1991) 205, 283); (Mazurkiewicz-Kwilecki and Nsonwah, Can. J. Physiol. Pharmacol. (1989) 67, 75-78); (Haas et al., Behav. Brain Res. (1995) 66, 41-44); (De Almeida and Izquierdo, Arch. Int. Pharmacodyn. (1986) 283, 193-198); (Kamei et al., Psychopharmacology (1990) 102, 312-318); (Kamei and Sakata, Japan. J. Pharmacol. (1991) 57, 437-482); (Schwartz et al., Psychopharmacology; The fourth Generation of Progress, Bloom and Kupfer (eds.), Raven Press, New York, (1995) 3 97); (Shaywitz et al., Psychopharmacology (1984) 82, 73-77); (Dumery and Blozovski, Exp. Brain Res. (1987) 67, 61-69); (Tedford et al., J. Pharmacol. Exp. Ther. (1995) 275, 598-604); (Tedford et al., Soc. Neurosci. Abstr. (1996) 22, 22); (Yokoyama et al., Eur. J. Pharmacol. (1993) 234, 129); (Yokoyama and Iinuma, CNS Drugs (1996) 5, 321); (Onodera et al., Prog. Neurobiol. (1994) 42, 685); (Leurs and Timmerman, Prog. Drug Res. (1992) 39, 127); (The Histamine H3 Receptor, Leurs and Timmerman (ed.), Elsevier Science, Amsterdam, The Netherlands (1998); (Leurs et al., Trends in Pharm. Sci. (1998) 19, 177-183); (Phillips et al., Annual Reports in Medicinal Chemistry (1998) 33, 31-40); (Matsubara et al., Eur. J. Pharmacol. (1992) 224, 145); (Rouleau et al., J. Pharmacol. Exp. Ther. (1997) 281, 1085); (Adam Szelag, “Role of histamine H3-receptors in the proliferation of neoplastic cells in vitro”, Med. Sci. Monit., 4(5): 747-755, (1998)); (Fitzsimons, C., H. Duran, F. Labombarda, B. Molinari and E. Rivera, “Histamine receptors signalling in epidermal tumor cell lines with H-ras gene alterations”, Inflammation Res., 47 (Suppl. 1): S50-S51, (1998)); (R. Leurs, R. C. Vollinga and H. Timmerman, “The medicinal chemistry and therapeutic potentials of ligand of the histamine H3 receptor”, Progress in Drug Research 45: 170-165, (1995)); (R. Levi and N. C. E. Smith, “Histamine H3-receptors: A new frontier in myocardial ischemia”, J. Pharm. Exp. Ther., 292: 825-830, (2000)); (Hatta, E., K Yasuda and R. Levi, “Activation of histamine H3 receptors inhibits carrier-mediated norepinephrine release in a human model of protracted myocardial ischemia”, J. Pharm. Exp. Ther., 283: 494-500, (1997); (H. Yokoyama and K. Iinuma, “Histamine and Seizures: Implications for the treatment of epilepsy”, CNS Drugs, 5(5); 321-330, (1995)); (K. Hurukami, H. Yokoyama, K. Onodera, K. Iinuma and T. Watanabe, AQ-0 145, “A newly developed histamine H3 antagonist, decreased seizure susceptibility of electrically induced convulsions in mice”, Meth. Find. Exp. Clin. Pharmacol., 17(C): 70-73, (1995); (Delaunois A., Gustin P., Garbarg M., and Ansay M., “Modulation of acetylcholine, capsaicin and substance P effects by histamine H3 receptors in isolated perfused rabbit lungs”, European Journal of Pharmacology 277(2-3):243-50, (1995)); and (Dimitriadou, et al., “Functional relationship between mast cells and C— sensitive nerve fibres evidenced by histamine H3-receptor modulation in rat lung and spleen”, Clinical Science 87(2):151-63, (1994). Such diseases or conditions include cardiovascular disorders such as acute myocardial infarction; memory processes, dementia and cognition disorders such as Alzheimer's disease and attention-deficit hyperactivity disorder; neurological disorders such as Parkinson's disease, schizophrenia, depression, epilepsy, and seizures or convulsions; cancer such as cutaneous carcinoma,” medullary thyroid carcinoma and melanoma; respiratory disorders such as asthma; sleep disorders such as narcolepsy; vestibular dysfunction such as Meniere's disease; gastrointestinal disorders, inflammation, migraine, motion sickness, obesity, pain, and septic shock.

H3 receptor antagonists have also been previously described in, for example, WO 03/050099, WO 02/0769252, and WO 02/12224. The histamine H3 receptor (H3R) regulates the release of histamine and other neurotransmitters, including serotonin and acetylcholine. H3R is relatively neuron specific and inhibits the release of certain monoamines such as histamine. Selective antagonism of H3R raises brain histamine levels and inhibits such activities as food consumption while minimizing non-specific peripheral consequences. Antagonists of the receptor increase synthesis and release of cerebral histamine and other monoamines. By this mechanism, they induce a prolonged wakefulness, improved cognitive function, reduction in food intake and normalization of vestibular reflexes. Accordingly, the receptor is an important target for new therapeutics in Alzheimer disease, mood and attention adjustments, including attention deficit hyperactive disorder (ADHD), cognitive deficiencies, obesity, dizziness, schizophrenia, epilepsy, sleeping disorders, narcolepsy and motion sickness, and various forms of anxiety.

The majority of histamine H3 receptor antagonists to date resemble histamine in possessing an imidazole ring that may be substituted, as described, for example, in WO96/38142. Non-imidazole neuroactive compounds such as beta histamines (Arrang, Eur. J. Pharm. 1985, 111:72-84) demonstrated some histamine H3 receptor activity but with poor potency. EP 978512 and EP 0982300A2 disclose non-imidazole alkyamines as histamine H3 receptor antagonists. WO 02/12224 (Ortho McNeil Pharmaceuticals) describes non-imidazole bicyclic derivatives as histamine H3 receptor ligands. Other receptor antagonists have been described in WO02/32893 and WO02/06233.

This invention is directed to histamine-3 (H3) receptor antagonists of the invention useful for treating the conditions listed in the preceding paragraphs. The compounds of this invention are highly selective for the H3 receptor (vs. other histamine receptors), and possess remarkable drug disposition properties (pharmacokinetics). In particular, the compounds of this invention selectively distinguish H3R from the other receptor subtypes H1R, H2R. In view of the increased level of interest in histamine H3 receptor agonists, inverse agonists and antagonists in the art, novel compounds that interact with the histamine H3 receptor would be a highly desirable contribution to the art. The present invention provides such a contribution to the art being based on the finding that a novel class of biaryl amines has a high and specific affinity to the histamine H3 receptor.

### SUMMARY OF THE INVENTION

This invention is directed to a compound of the formula I 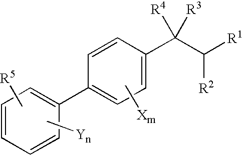
or a pharmaceutically acceptable salt thereof, wherein:
m=1, 2 or 3
n=1, 2, or 3
X and Y are independently selected from H, F, Cl, Br, I, C₁-C₆ alkyl (optionally substituted by F), C₁-C₆ alkoxyl (optionally substituted by F), (C₁-C₆ alkyl)-S(O)ₚ (optionally substituted by F, NO₂, COOH, COOR⁹, CONR¹⁰R¹¹;
wherein R⁹ is hydrogen, C₁-C₆ alkyl (optionally substituted by F), aryl, heteroaryl, C₁-C₆ alkyl-aryl, C₁-C₆alkyl-heteroaryl;
R¹⁰ and R¹¹ are chosen from the group consisting of hydrogen, C₁-C₆ alkyl, aryl, heteroaryl, C₁-C₆ alkyl-(aryl), or R¹⁰ and R¹¹ taken together with the nitrogen to which they are attached form a ring of 4-8 atoms with up to 3 additional heteroatoms including N, O, S; and
p=0, 1 or 2.
R¹ and R² are independently selected from the group consisting of
   hydrogen;
   C₁-C₈ alkyl optionally substituted with 1 to 4 halogens or OH;
   C₃-C₇ cycloalkyl;
   C₆-C₁₄ aryl;
   3-8-membered heterocycloalkyl optionally substituted with a C₁-C₄ alkyl-carbonyl group;
   C₆-C₁₀ arylsulfonyl optionally substituted with C₁-C₂ alkyl; and
   5-10-membered heteroaryl;
R³ is selected from the group consisting of
   C₁-C₈ alkyl optionally substituted with 1 to 4 halogens;
   C₃-C₇ cycloalkyl;
   C₆-C₁₄ aryl; or
R¹ and R² together with the nitrogen of the NR¹R² group form a 4-7 member ring, wherein one of the carbons in the ring is optionally replaced by O, S, NR⁶, or CO, and the ring is optionally fused to a C₆-C₁₀ arylene and is optionally substituted at a ring carbon with one or two C₁-C₄ alkyl groups, wherein R⁶ is
hydrogen;
C₁-C₈ alkyl optionally substituted with 1 to 4 halogens;
5-10-membered heteroaryl optionally substituted with a substituent selected from the group consisting of halogen, C₁-C₄ alkyl, C₁-C₂ alkoxy, C₆-C₁₀ aryl, C₁-C₄ alkylaminocarbonyl, cyano;
C₆-C₁₀ aryl optionally substituted with one or two C₁-C₂ alkyl; or
C₁-C₄ alkyl-carbonyl; or
R¹ and R³ together with the nitrogen of the NR¹R³ group form a 4-7 member ring, wherein one of the carbons in the ring is optionally replaced by O, S, NR⁶, or CO, and the ring is optionally fused to a C₆-C₁₀ arylene and is optionally substituted at a ring carbon with one or two C₁-C₄ alkyl groups, wherein R^{6′} is
hydrogen;
C₁-C₈ alkyl optionally substituted with 1 to 4 halogens;
5-10-membered heteroaryl optionally substituted with a substituent selected from the group consisting of halogen, C₁-C₄ alkyl, C₁-C₂ alkoxy, C₆-C₁₀ aryl, C₁-C₄ alkylaminocarbonyl, cyano;
C₆-C₁₀ aryl optionally substituted with one or two C₁-C₂ alkyl; or
C₁-C₄ alkyl-carbonyl;
R⁴ is
hydrogen, or
C₁-C₈ alkyl optionally substituted with 1 to 4 halogens;
R⁵ is (CH)ₜ—W, wherein W is a 5-7 member heteroaryl or heterocycloalkyl ring, optionally substituted by one or more substituents R⁷ and optionally fused to an aryl, 5-10-membered heteroaryl or C₄-C₈ cycloalkyl ring and wherein R⁷ is selected from the group consisting of
hydrogen;
F, Cl, Br or I;
C₁-C₆ alkyl (optionally substituted by F);
C₁-C₆alkoxyl (optionally substituted by F);
(C₁-C₆ alkyl)-S(O)ₚ (optionally substituted by F);
NO₂; NH2, NHR1′, NR1′R2′, wherein R¹ and R² are independently as defined in R¹ and R² above;
COOH, COOR^{9′}, CONR^{10′}R^{11′}, wherein R^{9′}, R^{10′} and R^{11′} are as independently defined in R⁹, R¹⁰ and R¹¹ above, and
t is 0, 1 or 2.

Where cis and trans isomers are possible for an embodiment of the inventive compound of formula I, both cis and trans isomers are within the scope of the invention.

The term “alkyl” refers to straight or branched chains of carbon atoms. Exemplary alkyl groups are C₁-C₆ alkyl groups which include methyl, ethyl, propyl, isopropyl, butyl, isobutyl, pentyl, isopentyl, hexyl, and the like, including all regioisomeric forms thereof, and straight and branched chain forms thereof. The term “alkyl” is also used to denote straight or branched chains of carbon atoms having one or more carbon-carbon double bonds, such as vinyl, allyl, butenyl, and the like, as well as straight or branched chains of carbon atoms having one or more carbon-carbon triple bonds, such as ethynyl, propargyl, butynyl, and the like. The term “aryl” denotes a cyclic, aromatic hydrocarbon. Examples of aryl groups include phenyl, naphthyl, anthracenyl, phenanthrenyl, and the like. The terms “alkoxy” and “aryloxy” denote “O-alkyl” and “O-aryl”, respectively. The term “cycloalkyl” denotes a cyclic group of carbon atoms, where the ring formed by the carbon atoms may be saturated or may comprise one or more carbon-carbon double bonds in the ring. Examples of cycloalkyl groups include cyclopropyl, cyclobutyl, cyclopentyl, cyclohexyl, cycloheptyl, and the like, as well as cyclopentenyl, cyclopentadienyl, cyclohexenyl, cyclohexadienyl, cyclobutadienyl, and the like. As used herein, the term “cycloalkyl” is also intended to denote a cyclic group comprising at least two fused rings, such as adamantanyl, decahydronaphthalinyl, norbornanyl, where the cyclic group may also have one or more carbon-carbon double bonds in one or both rings, such as in bicyclo[4.3.0]nona-3,6(1)-dienyl, dicyclopentadienyl, 1,2,3,4-tetrahydronaphthalinyl (tetralinyl), indenyl, and the like. The term “halogen” represents chloro, fluoro, bromo, and iodo. The term “heteroaryl” denotes a monocyclic or bicyclic aromatic group wherein one or more carbon atoms are replaced with heteroatoms selected from the group consisting of nitrogen, oxygen, and sulfur. If the heteroaryl group contains more than one heteroatom, the heteroatoms may be the same or different. Preferred heteroaryl groups are five- and six-member rings that contain from one to three heteroatoms independently selected from oxygen, nitrogen, and sulfur. Examples of preferred five- and six-member heteroaryl groups include benzo[b]thienyl, chromenyl, furyl, imidazolyl, indazolyl, indolizinyl, indolyl, isobenzofuranyl, isoindolyl, isoquinolyl, isothiazolyl, isoxazolyl, naphthyridinyl, oxadiazolyl, oxazinyl, oxazolyl, phthalazinyl, pteridinyl, purinyl, pyranyl, pyrazinyl, pyrazolyl, pyridazinyl, pyridyl, pyrimidyl, pyrrolyl, quinolizinyl, quinolyl, quinoxalinyl, thiazolyl, thienyl, triazinyl, triazolyl, and xanthenyl.

The term “heterocycloalkyl” denotes a cycloalkyl system, wherein “cycloalkyl” is defined above, in which one or more of the ring carbon atoms are replaced with a heteroatom selected from the group consisting of nitrogen, oxygen, and sulfur. Examples of such heterocycloalkyl groups include azabicycloheptanyl, azetidinyl, benzazepinyl, 1,3-dihydroisoindolyl, indolinyl, tetrahydrofuryl, tetrahydroquinolinyl, tetrahydroisoquinolinyl, morpholinyl, piperazinyl, piperidyl, pyrrolidinyl, and, tetrahydro-2H-1,4-thiazinyl.

A cyclic group may be bonded to another group in more than one way. If no particular bonding arrangement is specified, then all possible arrangements are intended. For example, the term “pyridyl” includes 2-, 3-, or 4-pyridyl, and the term “thienyl” includes 2- or 3-thienyl.

The term “C₀-C₄” includes the embodiment where there are no carbons in a chain. Thus, for example, the groups “C₃-C₇ cycloalkyl-C₀-C₄ alkyl,” “C₆-C₁₄ aryl-C₀-C₄ alkyl,” “5-10-membered heteroaryl-C₀-C₄ alkyl,” and “C₆-C₁₄ aryl-C₀-C₄ alkylene-O—C₀-C₄ alkyl” include C₃-C₇ cycloalkyl, C₆-C₁₄ aryl, 5-10-membered heteroaryl, and C₆-C₁₄ aryl- O—C₀-C₄ alkyl, respectively.

The term “C₁-C₄ dialkylamino” refers to a dialkylamino group in which each alkyl group is independently a C₁-C₄ alkyl group.

This invention is also directed to:
a pharmaceutical composition for treating, for example, a disorder or condition that may be treated by antagonizing histamine-3 receptors, the composition comprising a compound of formula I as described above, and optionally a pharmaceutically acceptable carrier;
a method of treatment of a disorder or condition that may be treated by antagonizing histamine-3 receptors, the method comprising administering to a mammal in need of such treatment a compound of formula I as described above; and
a pharmaceutical composition for treating, for example, a disorder or condition selected from the group consisting of depression, mood disorders, schizophrenia, anxiety disorders, Alzheimer's disease, attention-deficit disorder (ADD), attention-deficit hyperactivity disorder (ADHD), psychotic disorders, sleep disorders, obesity, dizziness, epilepsy, motion sickness, respiratory diseases, allergy, allergy-induced airway responses, allergic rhinitis, nasal congestion, allergic congestion, congestion, hypotension, cardiovascular disease, diseases of the GI tract, hyper and hypo motility and acidic secretion of the gastro-intestinal tract, the composition comprising a compound of formula I as described above, and optionally a pharmaceutically acceptable carrier.

This invention is also directed to a method of treatment of a disorder or condition selected from the group consisting of the disorders or conditions listed in the preceding paragraph, the method comprising administering to a mammal in need of such treatment a compound of formula I as described above.

The histamine-3 (H3) receptor antagonists of the invention are useful for treating, in particular, ADD, ADHD, obesity, anxiety disorders and respiratory diseases. Respiratory diseases that may be treated by the present invention include adult respiratory distress syndrome, acute respiratory distress syndrome, bronchitis, chronic bronchitis, chronic obstructive pulmonary disease, cystic fibrosis, asthma, emphysema, rhinitis and chronic sinusitis.

The pharmaceutical composition and method of this invention may also be used for preventing a relapse in a disorder or condition described in the previous paragraphs. Preventing such relapse is accomplished by administering to a mammal in need of such prevention a compound of formula I as described above.

The disclosed compounds may also be used as part of a combination therapy, including their administration as separate entities or combined in a single delivery system, which employs an effective dose of a histamine H3 antagonist compound of general formula I and an effective dose of a histamine H1 antagonist, such as cetirizine (Zyrtec™), for the treatment of allergic rhinitis, nasal congestion and allergic congestion.

The disclosed compounds may also be used as part of a combination therapy, including their administration as a separate entities or combined in a single delivery system, which employs an effective dose of a histamine H3 antagonist compound of general formula I and an effective dose of a neurotransmitter reuptake blocker. Examples of neurotransmitter reuptake blockers will include the serotonin-selective reuptake inhibitors (SSRI's) like sertraline (Zoloft™), fluoxetine (Prozac™), and paroxetine (Paxil™), or non-selective serotonin, dopamine or norepinephrine reuptake inhibitors for treating depression and mood disorders.

The compounds of the present invention may have optical centers and therefore may occur in different enantiomeric configurations. Formula I, as depicted above, includes all enantiomers, diastereomers, and other stereoisomers of the compounds depicted in structural formula I, as well as racemic and other mixtures thereof. Individual isomers can be obtained by known methods, such as optical resolution, optically selective reaction, or chromatographic separation in the preparation of the final product or its intermediate.

The present invention also includes isotopically labeled compounds, which are identical to those recited in formula I, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14; i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of formula I of this invention and prodrugs thereof can generally be prepared by carrying out the procedures disclosed in the Schemes and/or in the Examples and Preparations below, by substituting a readily available isotopically labeled reagent for a non-isotopically labeled reagent.

“Antagonizing histamine-3 (H3) receptors,” as used herein, refers to acting as a histamine-3 receptor antagonist.

A “unit dosage form” as used herein is any form that contains a unit dose of the compound of formula I. A unit dosage form may be, for example, in the form of a tablet or a capsule. The unit dosage form may also be in liquid form, such as a solution or suspension.

The compositions of the present invention may be formulated in a conventional manner using one or more pharmaceutically acceptable carriers. Thus, the active compounds of the invention may be formulated for oral, buccal, intranasal, parenteral (e.g., intravenous, intramuscular or subcutaneous) or rectal administration or in a form suitable for administration by inhalation or insufflation.

For oral administration, the pharmaceutical compositions may take the form of, for example, tablets or capsules prepared by conventional means with pharmaceutically acceptable excipients such as binding agents (e.g., pre-gelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

For buccal administration, the composition may take the form of tablets or lozenges formulated in conventional manner.

The active compounds of the invention may be formulated for parenteral administration by injection, including using conventional catheterization techniques or infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g., sterile pyrogen-free water, before use.

The active compounds of the invention may also be formulated in rectal compositions such as suppositories or retention enemas, e.g., containing conventional suppository bases such as cocoa butter or other glycerides.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch.

A proposed dose of the active compounds of the invention for oral, parenteral or buccal administration to the average adult human for the treatment of the conditions referred to above (e.g., depression) is 0.1 to 200 mg of the active ingredient per unit dose which could be administered, for example, 1 to 4 times per day.

Aerosol formulations for treatment of the conditions referred to above (e.g., attention deficit hyperactivity disorder) in the average human are preferably arranged so that each metered dose or “puff” of aerosol contains 20 μg to 1000 μg of the compound of the invention. The overall daily dose with an aerosol will be within the range 100 μg to 10 mg. Administration may be several times daily, for example 2, 3, 4 or 8 times, giving for example, 1, 2 or 3 doses each time.

In connection with the use of an active compound of this invention with a histamine H1 antagonist, preferably cetirizine, for the treatment of subjects possessing any of the above conditions, it is to be noted that these compounds may be administered either alone or in combination with pharmaceutically acceptable carriers by either of the routes previously indicated, and that such administration can be carried out in both single and multiple dosages. More particularly, the active combination can be administered in a wide variety of different dosage forms, i.e., they may be combined with various pharmaceutically-acceptable inert carriers in the form of tablets, capsules, lozenges, troches, hard candies, powders, sprays, aqueous suspension, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes. In general, the compounds of formula I are present in such dosage forms at concentration levels ranging from about 0.5% to about 95% by weight of the total composition, i.e., in amounts which are sufficient to provide the desired unit dosage and a histamine H1 antagonist, preferably cetirizine, is present in such dosage forms at concentration levels ranging from about 0.5% to about 95% by weight of the total composition, i.e., in amounts which are sufficient to provide the desired unit dosage.

A proposed daily dose of an active compound of this invention in the combination formulation (a formulation containing an active compound of this invention and a histamine H1 antagonist) for oral, parenteral, rectal or buccal administration to the average adult human for the treatment of the conditions referred to above is from about 0.01 mg to about 2000 mg, preferably from about 0.1 mg to about 200 mg of the active ingredient of formula I per unit dose which could be administered, for example, 1 to 4 times per day.

A proposed daily dose of a histamine H1 antagonist, preferably cetirizine, in the combination formulation for oral, parenteral or buccal administration to the average adult human for the treatment of the conditions referred to above is from about 0.1 mg to about 2000 mg, preferably from about 1 mg to about 200 mg of the histamine H1 antagonist per unit dose which could be administered, for example, 1 to 4 times per day.

A preferred dose ratio of cetirizine to an active compound of this invention in the combination formulation for oral, parenteral or buccal administration to the average adult human for the treatment of the conditions referred to above is from about 0.00005 to about 20,000, preferably from about 0.25 to about 2,000.

Aerosol combination formulations for treatment of the conditions referred to above in the average adult human are preferably arranged so that each metered dose or “puff” of aerosol contains from about 0.01 μg to about 100 mg of the active compound of this invention, preferably from about 1 μg to about 10 mg of such compound. Administration may be several times daily, for example 2, 3, 4 or 8 times, giving for example, 1, 2 or 3 doses each time.

Aerosol formulations for treatment of the conditions referred to above in the average adult human are preferably arranged so that each metered dose or “puff” of aerosol contains from about 0.01 mg to about 2000 mg of a histamine H1 antagonist, preferably cetirizine, preferably from about 1 mg to about 200 mg of cetirizine. Administration may be several times daily, for example 2, 3, 4 or 8 times, giving for example, 1, 2 or 3 doses each time.

As previously indicated, a histamine H1 antagonist, preferably cetirizine, in combination with compounds of formula I are readily adapted to therapeutic use as antidepressant agents. In general, these antidepressant compositions containing a histamine H1 antagonist, preferably cetirizine, and a compound of formula I are normally administered in dosages ranging from about 0.01 mg to about 100 mg per kg of body weight per day of a histamine H1 antagonist, preferably cetirizine, preferably from about 0.1 mg. to about 10 mg per kg of body weight per day of cetirizine; with from about 0.001 mg. to about 100 mg per kg of body weight per day of a compound of formula I, preferably from about 0.01 mg to about 10 mg per kg of body weight per day of a compound of formula I, although variations will necessarily occur depending upon the conditions of the subject being treated and the particular route of administration chosen.

In connection with the use of an active compound of this invention with a neurotransmitter re-uptake blocker, preferably sertraline, for the treatment of subjects possessing any of the above conditions, it is to be noted that these compounds may be administered either alone or in combination with pharmaceutically acceptable carriers by either of the routes previously indicated, and that such administration can be carried out in both single and multiple dosages. More particularly, the active combination can be administered in a wide variety of different dosage forms, i.e., they may be combined with various pharmaceutically-acceptable inert carriers in the form of tablets, capsules, lozenges, troches, hard candies, powders, sprays, aqueous suspension, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes. In general, the compounds of formula I are present in such dosage forms at concentration levels ranging from about 0.5% to about 95% by weight of the total composition, i.e., in amounts which are sufficient to provide the desired unit dosage and a neurotransmitter re-uptake blocker, preferably sertraline, is present in such dosage forms at concentration levels ranging from about 0.5% to about 95% by weight of the total composition, i.e., in amounts which are sufficient to provide the desired unit dosage.

A proposed daily dose of an active compound of this invention in the combination formulation (a formulation containing an active compound of this invention and a SSRI re-uptake inhibitor) for oral, parenteral, rectal or buccal administration to the average adult human for the treatment of the conditions referred to above is from about 0.01 mg to about 2000 mg, preferably from about 0.1 mg to about 200 mg of the active ingredient of formula I per unit dose which could be administered, for example, 1 to 4 times per day.

A proposed daily dose of a neurotransmitter re-uptake blocker, preferably sertraline, in the combination formulation for oral, parenteral or buccal administration to the average adult human for the treatment of the conditions referred to above is from about 0.1 mg to about 2000 mg, preferably from about 1 mg to about 200 mg of the neurotransmitter re-uptake blocker per unit dose which could be administered, for example, 1 to 4 times per day.

A preferred dose ratio of sertraline to an active compound of this invention in the combination formulation for oral, parenteral or buccal administration to the average adult human for the treatment of the conditions referred to above is from about 0.00005 to about 20,000, preferably from about 0.25 to about 2,000.

Aerosol combination formulations for treatment of the conditions referred to above in the average adult human are preferably arranged so that each metered dose or “puff” of aerosol contains from about 0.01 μg to about 100 mg of the active compound of this invention, preferably from about 1 μg to about 10 mg of such compound. Administration may be several times daily, for example 2, 3, 4 or 8 times, giving for example, 1, 2 or 3 doses each time.

Aerosol formulations for treatment of the conditions referred to above in the average adult human are preferably arranged so that each metered dose or “puff” of aerosol contains from about 0.01 mg to about 2000 mg of a neurotransmitter re-uptake blocker, preferably sertraline, preferably from about 1 mg to about 200 mg of sertraline. Administration may be several times daily, for example 2, 3, 4 or 8 times, giving for example, 1, 2 or 3 doses each time.

As previously indicated, a neurotransmitter re-uptake blocker, preferably sertraline, in combination with compounds of formula I are readily adapted to therapeutic use as antidepressant agents. In general, these antidepressant compositions containing a neurotransmitter re-uptake blocker, preferably sertraline, and a compound of formula I are normally administered in dosages ranging from about 0.01 mg to about 100 mg per kg of body weight per day of a neurotransmitter re-uptake blocker, preferably sertraline, preferably from about 0.1 mg. to about 10 mg per kg of body weight per day of sertraline; with from about 0.001 mg. to about 100 mg per kg of body weight per day of a compound of formula I, preferably from about 0.01 mg to about 10 mg per kg of body weight per day of a compound of formula I, although variations will necessarily occur depending upon the conditions of the subject being treated and the particular route of administration chosen.

Anxiety disorders include, for example, generalized anxiety disorder, panic disorder, PTSD, and social anxiety disorder. Mood adjustment disorders include, for example, depressed mood, mixed anxiety and depressed mood, disturbance of conduct, and mixed disturbance of conduct and depressed mood. Attention adjustment disorders include, for example, in addition to ADHD, attention-deficit disorders or other cognitive disorders due to general medical conditions. Psychotic disorders include, for example, schizoaffective disorders and schizophrenia; sleep disorders include, for example, narcolepsy and enuresis.

Examples of the disorders or conditions which may be treated by the compound, composition and method of this invention are also as follows: depression, including, for example, depression in cancer patients, depression in Parkinson's patients, post-myocardial Infarction depression, depression in patients with human immunodeficiency virus (HIV), Subsyndromal Symptomatic depression, depression in infertile women, pediatric depression, major depression, single episode depression, recurrent depression, child abuse induced depression, post partum depression, DSM-IV major depression, treatment-refractory major depression, severe depression, psychotic depression, post-stroke depression, neuropathic pain, manic depressive illness, including manic depressive illness with mixed episodes and manic depressive illness with depressive episodes, seasonal affective disorder, bipolar depression BP I, bipolar depression BP II, or major depression with dysthymia; dysthymia; phobias, including, for example, agoraphobia, social phobia or simple phobias; eating disorders, including, for example, anorexia nervosa or bulimia nervosa; chemical dependencies, including, for example, addictions to alcohol, cocaine, amphetamine and other psychostimulants, morphine, heroin and other opioid agonists, phenobarbital and other barbiturates, nicotine, diazepam, benzodiazepines and other psychoactive substances; Parkinson's diseases, including, for example, dementia in Parkinson's disease, neuroleptic-induced parkinsonism or tardive dyskinesias; headache, including, for example, headache associated with vascular disorders; withdrawal syndrome; age-associated learning and mental disorders; apathy; bipolar disorder; chronic fatigue syndrome; chronic or acute stress; conduct disorder; cyclothymic disorder; somatoform disorders such as somatization disorder, conversion disorder, pain disorder, hypochondriasis, body dysmorphic disorder, undifferentiated disorder, and somatoform NOS; incontinence; inhalation disorders; intoxication disorders; mania; oppositional defiant disorder; peripheral neuropathy; post-traumatic stress disorder; late luteal phase dysphoric disorder; specific developmental disorders; SSRI “poop out” syndrome, or a patient's failure to maintain a satisfactory response to SSRI therapy after an initial period of satisfactory response; and tic disorders including Tourette's disease.

As an example, the mammal in need of the treatment or prevention may be a human. As another example, the mammal in need of the treatment or prevention may be a mammal other than a human.

A compound of formula I that is basic in nature is capable of forming a wide variety of different salts with various inorganic and organic acids. The acid addition salts are readily prepared by treating the base compounds with a substantially equivalent amount of the chosen mineral or organic acid in an aqueous solvent medium or in a suitable organic solvent such as methanol or ethanol. Upon careful evaporation of the solvent, the desired solid salt is obtained.

The acids which are used to prepare the pharmaceutically acceptable acid salts of the active compound used in formulating the pharmaceutical composition of this invention that are basic in nature are those which form non-toxic acid addition salts, i.e., salts containing pharmacologically acceptable anions. Non-limiting examples of the salts include the acetate, benzoate, beta-hydroxybutyrate, bisulfate, bisulfite, bromide, butyne-1,4-dioate, caproate, chloride, chlorobenzoate, citrate, dihydrogenphosphate, dinitrobenzoate, fumarate, glycollate, heptanoate, hexyne-1,6-dioate, hydroxybenzoate, iodide, lactate, maleate, malonate, mandelate, metaphosphate, methanesulfonate, methoxybenzoate, methylbenzoate, monohydrogen phosphate, naphthalene-1-sulfonate, naphthalene-2-sulfonate, oxalate, phenylbutyrate, phenylpropionate, phosphate, phthalate, phenylacetate, propanesulfonate, propiolate, propionate, pyrophosphate, pyrosulfate, sebacate, suberate, succinate, sulfate, sulfite, sulfonate, tartrate, xylenesulfonate, acid phosphate, acid citrate, bitartrate, succinate, gluconate, saccharate, nitrate, methanesulfonate and pamoate [i.e., 1,1′-methylene-bis-(2-hydroxy-3-naphthoate)] salts.

Preferred embodiments of the present invention include the compounds of formula I in which
(A) R¹ is methyl, R² is methyl and R³ is methyl; or
(B) R¹ and R² together with the nitrogen to which they are attached form the 5-membered pyrrolidine ring, and R is methyl; or
(C)R¹ and R³ together with the nitrogen to which they are attached form a 5-membered pyrrolidine ring, and R is methyl; or
(D) R¹ and R² together with the nitrogen to which they are attached form the 6-membered piperidine ring, and R³ is methyl; or
(E) R¹ and R³ together with the nitrogen to which they are attached form the 6-membered piperidine ring, and R² is methyl.

The most preferred embodiment of the present invention include the compounds of formula I in which R¹ and R² together with nitrogen to which they are attached form the 5-membered pyrrolidine ring and R³ is methyl.

Preferred embodiments of the present invention also include any combination of the foregoing embodiments (A)-(E).

Preferred compounds of formula I in accordance with the present invention are the following:
(R)-3-[4′-(1-Pyrrolidin-1-ylethyl)-biphenyl-4-yl]-piperidine,
(±)-5-[4′-(1-Pyrrolidin-1-ylethyl)-biphenyl-4-yl]-pyrimidine,
(R)-4-[4′-(1-Pyrrolidin-1-ylethyl)-biphenyl-4-yl]-piperidine,
(S)-4-[4′-(1-Pyrrolidin-1-ylethyl)-biphenyl-4-yl]-piperidine,
(±)-4-[4′-(1-Pyrrolidin-1-ylethyl)-biphenyl-4-yl]-piperidine,
(±)-3-[4′-(1-Pyrrolidin-1-ylethyl)-biphenyl-4-yl]-pyridine,
(R)-2-[4′-(1-Pyrrolidin-1-ylethyl)-biphenyl-4-yl]-piperidine,
(±)-Dimethyl-[1-(4′-pyridin-4-yl-biphenyl-4-yl)-ethyl]-amine,
(R)-5-[4′-(1-Pyrrolidin-1-ylethyl)-biphenyl-4-yl]-pyrimidine,
(S)-5-[4′-(1-Pyrrolidin-1-ylethyl)-biphenyl-4-yl]-pyrimidine,
(R)-4-[4′-(1-Pyrrolidin-1-ylethyl)-biphenyl-4-yl]-pyridine,
(R)-3-[4′-(1-Pyrrolidin-1-ylethyl)-biphenyl-4-yl]-pyridine,
(±)-1-[1-(4′-Benzo[b]thiophen-2-ylbiphenyl-4-yl)-ethyl]-pyrrolidine,
(±)-4-(1-Pyrrolidin-1-ylethyl)-[1,1′;4′,1″]terphenyl-3″-carbonitrile,
(±)-3,5-Dimethyl-4-[4′-(1-pyrrolidin-1-ylethyl)-biphenyl-4-yl]-isoxazole,
(±)-4″-(1-Pyrrolidin-1-ylethyl)-[1,1′;4′,1″]terphenyl-3-carboxylic acid dimethylamide,
(±)-1-{1-[4′-(2-Phenylcyclopropyl)-biphenyl-4-yl]-ethyl}-pyrrolidine,
(±)-3-Chloro-4-[4′-(1-pyrrolidin-1-ylethyl)-biphenyl-4-yl]-pyridine,
(±)-1-[1-(3″-Methylsulfanyl-[1,1′;4′,1″]terphenyl-4-yl)-ethyl]-pyrrolidine,
(±)-1-{1-[4′-(2,3-Dihydro-benzo[1,4]dioxin-6-yl)-biphenyl-4-yl]-ethyl}-pyrrolidine,
(±)-4″-(1-Pyrrolidin-1-ylethyl)-[1,1′;4′,1″]terphenyl-3-carboxylic acid amide,
(±)-3-Fluoro-4-[4′-(1-pyrrolidin-1-ylethyl)-biphenyl-4-yl]-pyridine,
(±)-5-[4′-(1-Pyrrolidin-1-ylethyl)-biphenyl-4-yl]-pyrimidine,
(±)-1-Methyl-5-[4′-(1-pyrrolidin-1-ylethyl)-biphenyl-4-yl]-1H-indole,
(±)-1-[1-(4′-Benzo[b]thiophen-3-ylbiphenyl-4-yl)-ethyl]-pyrrolidine,
(±)-4″-(1-Pyrrolidin-1-ylethyl)-[1,1′;4′,1″]terphenyl-2-sulfonic acid tert-butyl-amide,
(S)-4-[4′-(1-Pyrrolidin-1-ylethyl)-biphenyl-4-yl]-pyridine,
(±)-1-[1-(4′-Furan-2-ylbiphenyl-4-yl)-ethyl]-pyrrolidine,
(±)-1-[1-(4′-Benzo[1,3]dioxol-5-ylbiphenyl-4-yl)-ethyl]-pyrrolidine,
(±)-5-[4′-(1-Pyrrolidin-1-ylethyl)-biphenyl-4-yl]-isoquinoline,
(±)-4″-(1-Pyrrolidin-1-ylethyl)-[1,1′;4′,1″]terphenyl-2-carboxylic acid diisopropylamide,
(±)-[4-(1-Pyrrolidin-1-ylethyl)-[1,1′;4′,1″]terphenyl-4″-yl]-methanol,
(±)-[4-(1-Pyrrolidin-1-ylethyl)-[1,1′;4′,1″]terphenyl-3″-yl]-methanol, (±)-[4″-(1-Pyrrolidin-1-ylethyl)-[1,1′;4′,1″]terphenyl-2-yl]-methanol,
(±)-1-[4″-(1-Pyrrolidin-1-ylethyl)-[1,1′;4′,1″]terphenyl-3-yl]-1H-pyrazole,
(±)-N-[4″-(1-Pyrrolidin-1-ylethyl)-[1,1′;4′,1″]terphenyl-3-yl]-acetamide,
(±)-4-(1-Pyrrolidin-1-ylethyl)-[1,1′;4′,1″]terphenyl-4″-carbonitrile,
(±)-1-[1-(4-Methanesulfonyl-[1,1′;4′,1″]terphenyl-4″-yl)-ethyl]-pyrrolidine,
(±)-1-[1-(3,5-Dichloro-[1,1′;4′,1″]terphenyl-4″-yl)-ethyl]-pyrrolidine,
(±)-1-[1-(3″,4″-Dichloro-[1,1′;4′,1″]terphenyl-4-yl)-ethyl]-pyrrolidine,
(±)-1-[1-(4′-Thiophen-3-ylbiphenyl-4-yl)-ethyl]-pyrrolidine,
(±)-{1-[4′-(3-Fluoro-pyridin-4-yl)-biphenyl-4-yl]-ethyl}-dimethylamine,
(±)-{1-[4′-(2,3-Dihydro-benzo[1,4]dioxin-6-yl)-biphenyl-4-yl]-ethyl}-dimethylamine,
(±)-Dimethyl-{1-[4′-(1-methyl-1H-indol-5-yl)-biphenyl-4-yl]-ethyl}-amine,
(±)-[1-(4′-Benzo[b]thiophen-3-ylbiphenyl-4-yl)-ethyl]-dimethylamine,
(±)-4″-(1-Dimethylaminoethyl)-[1,1′;4′,1″]terphenyl-2-sulfonic acid tert-butyl-amide,
(±)-4″-(1-Dimethylaminoethyl)-[1,1′;4′,1″]terphenyl-3-carbonitrile,
(±)-4″-(1-Dimethylaminoethyl)-3-methoxy-[1,1′;4′,1″]terphenyl-2-carboxylic acid diisopropylamide,
(±)-{1-[4′-(3,5-Dimethylisoxazol-4-yl)-biphenyl-4-yl]-ethyl}-dimethylamine,
(±)-4″-(1-Dimethylaminoethyl)-[1,1′;4′,1″]terphenyl-2-carboxylic acid diisopropylamide,
(±)-Dimethyl-[1-(4′-thiophen-2-ylbiphenyl-4-yl)-ethyl]-amine,
(±)-Dimethyl-[1-(4′-thiophen-3-ylbiphenyl-4-yl)-ethyl]-amine,
(±)-[1-(4′-Benzofuran-2-ylbiphenyl-4-yl)-ethyl]-dimethylamine,
(±)-[4-(1-Dimethylaminoethyl)-[1,1′;4′,1″]terphenyl-4″-yl]-methanol,
(±)-[1-(4′-Furan-2-ylbiphenyl-4-yl)-ethyl]-dimethylamine,
(±)-[1-(4′-Benzo[1,3]dioxol-5-ylbiphenyl-4-yl)-ethyl]-dimethylamine,
(±)-[4″-(1-Dimethylaminoethyl)-[1,1′;4′,1″]terphenyl-3-yl]-methanol,
(±)-[4″-(1-Dimethylaminoethyl)-[1,1′;4′,1″]terphenyl-2-yl]-methanol,
(±)-Dimethyl-[1-(4′-pyridin-4-ylbiphenyl-4-yl)-ethyl]-amine,
(±)-[1-(4′-Furan-3-ylbiphenyl-4-yl)-ethyl]-dimethylamine,
(±)-N-[4″-(1-Dimethylaminoethyl)-[1,1′;4′,1″]terphenyl-3-yl]-acetamide,
(±)-Dimethyl-[1-(2-methylsulfanyl-[1,1′;4′,1″]terphenyl-4″-yl)-ethyl]-amine,
(±)-4-(1-Dimethylaminoethyl)-[1,1′;4′,1″]terphenyl-4″-carbonitrile,
(±)-[1-(4-Methanesulfonyl-[1,1′;4′,1″]terphenyl-4″-yl)-ethyl]-dimethylamine,
(±)-[1-(4-Ethanesulfonyl-[1,1′;4′,1″]terphenyl-4″-yl)-ethyl]-dimethylamine,
(±)-[1-(4′-Isoquinolin-5-ylbiphenyl-4-yl)-ethyl]-dimethylamine,
(±)-Dimethyl-[1-(3-pyrazol-1-yl-[1,1′;4′,1″]terphenyl-4″-yl)-ethyl]-amine,
(±)-Dimethyl-[1-(3-methylsulfanyl-[1,1′;4′,1″]terphenyl-4″-yl)-ethyl]-amine,
(±)-{1-[4′-(3-Chloropyridin-4-yl)-biphenyl-4-yl]-ethyl}-dimethylamine,
(±)-Dimethyl-[1-(4′-pyrimidin-5-ylbiphenyl-4-yl)-ethyl]-amine,
(±)-{1-[4′-(2,4-Dimethoxypyrimidin-5-yl)-biphenyl-4-yl]-ethyl}-dimethylamine,
(R)-2-[4′-(1-Pyrrolidin-1-ylethyl)-biphenyl-4-yl]-pyridine,
(±)-2-[4′-(1-Pyrrolidin-1-ylethyl)-biphenyl-4-yl]-piperidine,
(R)-1-Methyl-4-[4′-(1-pyrrolidin-1-ylethyl)-biphenyl-4-yl]-piperidine,
(R)-1-Ethyl-4-[4′-(1-pyrrolidin-1-ylethyl)-biphenyl-4-yl]-piperidine,
(±)-1-[2′-Methyl-4′-(1-methylpyrrolidin-2-yl)-biphenyl-4-ylmethyl]-piperidine, and
(R)-2,4-Dimethoxy-5-[4′-(1-pyrrolidin-1-ylethyl)-biphenyl-4-yl]-pyrimidine,
1-Methanesulfonyl-4-[4′-(1-pyrrolidin-1-ylethyl)-biphenyl-4-yl]-piperidine,
5-[3,5-Difluoro-4′-(1-pyrrolidin-1-ylethyl)-biphenyl-4-yl]-pyrimidin-2-ol,
5-[2,5-Difluoro-4′-(1-pyrrolidin-1-ylethyl)-biphenyl-4-yl]-pyrimidin-2-ol,
5-[3-Fluoro-4′-(1-pyrrolidin-1-ylethyl)-biphenyl-4-yl]-pyrimidin-2-ol,
5-[3,5-Difluoro-4′-(1-pyrrolidin-1-ylethyl)-biphenyl-4-yl]-pyrimidine,
5-[3,5-Difluoro-4′-(1-pyrrolidin-1-ylethyl)-biphenyl-4-yl]-2-methoxypyrimidine,
5-[2,5-Difluoro-4′-(1-pyrrolidin-1-ylethyl)-biphenyl-4-yl]-pyrimidine,
5-[2,5-Difluoro-4′-(1-pyrrolidin-1-ylethyl)-biphenyl-4-yl]-2-methoxy-pyrimidine,
5-[3-Fluoro-4′-(1-pyrrolidin-1-ylethyl)-biphenyl-4-yl]-pyrimidine,
5-[3-Fluoro-4′-(1-pyrrolidin-1-ylethyl)-biphenyl-4-yl]-2-methoxypyrimidine,
5-[4′-(1-Pyrrolidin-1-ylethyl)-biphenyl-4-yl]-pyrimidin-2-ol,
2-Chloro-5-[4′-(1-pyrrolidin-1-ylethyl)-biphenyl-4-yl]-pyrimidine,
2-Methoxy-5-[4′-(1-pyrrolidin-1-ylethyl)-biphenyl-4-yl]-pyrimidine,
5-[4′-(1-Pyrrolidin-1-ylethyl)-biphenyl-4-yl]-pyrimidin-2-ylamine,
5-[2-Fluoro-4′-(1-pyrrolidin-1-ylethyl)-biphenyl-4-yl]-pyrimidine,
2-[4′-(1-Pyrrolidin-1-ylethyl)-biphenyl-4-yl]-pyrimidine,
(4-Chlorobenzyl)-[2′-methyl-4′-(1-methylpyrrolidin-2-yl)-biphenyl-4-ylmethyl]-amine, and
[2′-Methyl-4′-(1-methylpyrrolidin-2-yl)-biphenyl-4-ylmethyl]-(1-methyl-2-morpholin-4-ylethyl)-amine.

The most preferred examples of compounds according to the present invention include:
1-[4′-(1-Pyrrolidin-1-ylethyl)-biphenyl-4-yl]-1H-pyrazole,
2-[4′-(1-Pyrrolidin-1-ylethyl)-biphenyl-4-yl]-pyrazine,
1-[4′-(1-Pyrrolidin-1-ylethyl)-biphenyl-4-yl]-1H-[1,2,4]triazole,
4-[4′-(1-Pyrrolidin-1-ylethyl)-biphenyl-4-yl]-4H-[1,2,4]triazole,
2,4-Dimethyl-1-[4′-(1-pyrrolidin-1-ylethyl)-biphenyl-4-yl]-1H-imidazole,
2-Methyl-5-[4′-(1-pyrrolidin-1-ylethyl)-biphenyl-4-yl]-pyrimidine,
2-Fluoro-5-[4′-(1-pyrrolidin-1-ylethyl)-biphenyl-4-yl]-pyrimidine,
2-Fluoro-4-methyl-5-[4′-(1-pyrrolidin-1-ylethyl)-biphenyl-4-yl]-pyrimidine,
5-[3-Methyl-4′-(1-pyrrolidin-1-ylethyl)-biphenyl-4-yl]-pyrimidine,
5-[3,5-Dimethyl-4′-(1-pyrrolidin-1-ylethyl)-biphenyl-4-yl]-pyrimidine,
2,6-Dimethyl-3-[4′-(1-pyrrolidin-1-ylethyl)-biphenyl-4-yl]-pyridine,
2-Methyl-5-[4′-(1-pyrrolidin-1-ylethyl)-biphenyl-4-yl]-pyridine,
5-{4′-[1-(2-Methyl-pyrrolidin-1-yl)-ethyl]-biphenyl-4-yl}-pyrimidine,
5-{4′-[1-(2,5-Dimethyl-pyrrolidin-1-yl)-ethyl]-biphenyl-4-yl}-pyrimidine,
5-{4′-[1-(2,2-Dimethyl-pyrrolidin-1-yl)-ethyl]-biphenyl-4-yl}-pyrimidine,
5-{4′-[1-(3,3-Dimethyl-pyrrolidin-1-yl)-ethyl]-biphenyl-4-yl}-pyrimidine,
5-[4′-(1-Piperidin-1-ylethyl)-biphenyl-4-yl]-pyrimidine,
4-[1-(4′-Pyrimidin-5-yl-biphenyl-4-yl)-ethyl]-morpholine,
5-[4′-(1-Methyl-piperidin-2-yl)-biphenyl-4-yl]-pyrimidine,
4-Methyl-3-(4′-pyrimidin-5-yl-biphenyl-4-yl)-morpholine,
5-[4′-(1,4-Dimethyl-piperazin-2-yl)-biphenyl-4-yl]-pyrimidine,
5-[4′-(1,5-Dimethyl-pyrrolidin-2-yl)-biphenyl-4-yl]-pyrimidine,
3-(4′-Pyrimidin-5-yl-biphenyl-4-yl)-octahydro-indolizine,
5-[4′-(1-Isopropyl-pyrrolidin-2-yl)-biphenyl-4-yl]-pyrimidine,
5-[4′-(1-Benzyl-pyrrolidin-2-yl)-biphenyl-4-yl]-pyrimidine,
5-[2′-Fluoro-4′-(1-methyl-pyrrolidin-2-yl)-biphenyl-4-yl]-pyrimidine,
5-[2′,6′-Difluoro-4′-(1-methyl-pyrrolidin-2-yl)-biphenyl-4-yl]-pyrimidine,
5-[2-Methyl-4′-(1-methyl-pyrrolidin-2-yl)-biphenyl-4-yl]-pyrimidine,
5-[4′-(1-Methyl-1-pyrrolidin-1-yl-ethyl)-biphenyl-4-yl]-pyrimidine,
2-Methyl-4-[4′-(1-pyrrolidin-1-yl-ethyl)-biphenyl-4-yl]-piperidine,
2,6-Dimethyl-4-[4′-(1-pyrrolidin-1-yl-ethyl)-biphenyl-4-yl]-piperidine,
1,2,6-Trimethyl-4-[4′-(1-pyrrolidin-1-yl-ethyl)-biphenyl-4-yl]-piperidine,
2-Methyl-6-[4′-(1-pyrrolidin-1-yl-ethyl)-biphenyl-4-yl]-piperidine,
3,6-Dimethyl-2-[4′-(1-pyrrolidin-1-yl-ethyl)-biphenyl-4-yl]-piperidine,
1,2-Dimethyl-6-[4′-(1-pyrrolidin-1-yl-ethyl)-biphenyl-4-yl]-piperidine,
1-[4′-(1-Methyl-pyrrolidin-2-yl)-biphenyl-4-ylmethyl]-pyrrolidine,
1-[4′-(1-Methyl-pyrrolidin-2-yl)-biphenyl-4-ylmethyl]-2methyl-pyrrolidine,
2-[4′-(1-Methyl-pyrrolidin-2-yl)-biphenyl-4-ylmethyl]-2,3-dihydro-1H-isoindole,
2-[4′-(1-Methyl-pyrrolidin-2-yl)-biphenyl-4-ylmethyl]-octahydro-isoindole,
1-[4′-(1-Methyl-pyrrolidin-2-yl)-biphenyl-4-ylmethyl]-1-aza-spiro[4.5]decane,
8-[4′-(1-Methyl-pyrrolidin-2-yl)-biphenyl-4-ylmethyl]-8-aza-bicyclo[3.2.1]octane,
2-[4′-(1-Methyl-pyrrolidin-2-yl)-biphenyl-4-ylmethyl]-2-aza-bicyclo[2.2.2]octane,
4-[4′-(1-Methyl-pyrrolidin-2-yl)-biphenyl-4-ylmethyl]-morpholine,
4-[4′-(1-Methyl-pyrrolidin-2-yl)-biphenyl-4-ylmethyl]-thiomorpholine,
4-[4′-(1-Methyl-pyrrolidin-2-yl)-biphenyl-4-ylmethyl]-thiomorpholine 1-oxide,
4-[4′-(1-Methyl-pyrrolidin-2-yl)-biphenyl-4-ylmethyl]-thiomorpholine 1,1-dioxide,
1-[4′-(1-Methyl-pyrrolidin-2-yl)-biphenyl-4-ylmethyl]-azepine,
Dicyclopropyl-[4′-(1-methyl-pyrrolidin-2-yl)-biphenyl-4-ylmethyl]-amine,
Methyl-[4′-(1-methyl-pyrrolidin-2-yl)-biphenyl-4-ylmethyl]-phenyl-amine,
1-[4′-(1-Methyl-pyrrolidin-2-yl)-biphenyl-4-ylmethyl]-2,3-dihydro-1H-indole,
3-[4′-(1-Methyl-pyrrolidin-2-yl)-biphenyl-4-ylmethyl]-2,3-dihydro-benzothiazole,
Cyclohexyl-methyl-[4′-(1-methyl-pyrrolidin-2-yl)-biphenyl-4-ylmethyl]-amine,
Methyl-[4′-(1-methyl-pyrrolidin-2-yl)-biphenyl-4-ylmethyl]-(tetrahydropyran-4-yl)-amine,
4-[4′-(1-Pyrrolidin-1-yl-propyl)-biphenyl-4-yl]-pyridine,
2-Methyl-5-[1-(4′-pyridin-4-yl biphenyl-4-yl)-ethyl]-octahydro-pyrrolo[3,4-c]pyrrole,
2-[1-(4′-Pyridin-4-ylbiphenyl-4-yl)-ethyl]-octahydro-isoindole,
(1-Azabicyclo[2.2.2]oct-3-yl)-[1-(4′-pyridin-4-ylbiphenyl-4-yl)-ethyl]-amine,
Dimethyl-[phenyl-(4′-pyridin-4-ylbiphenyl-4-yl)-methyl]-amine,
tert-Butyl-[1-(4′-pyridin-4-ylbiphenyl-4-yl)-ethyl]-amine,
tert-Butyl-methyl-[1-(4′-pyridin-4-ylbiphenyl-4-yl)-ethyl]-amine,
4-[4′-(1-Pyrrolidin-1-ylethyl)-biphenyl-4-yl]-4H-[1,2,4]triazole, and
1-[4′-(1-Pyrrolidin-1-ylethyl)-biphenyl-4-yl]-1H-imidazole.
5-[4′-(1-Piperidin-1-ylethyl)-biphenyl-4-yl]-pyrimidine,
2-Methyl-5-[4′-(1-piperidin-1-ylethyl)-biphenyl-4-yl]-pyrimidine,
5-{4′-[1-(2,6-Dimethylpiperidin-1-yl)-ethyl]-biphenyl-4-yl}-pyrimidine,
5-[2′-Methyl-4′-(1-piperidin-1-ylethyl)-biphenyl-4-yl]-pyrimidine,
2-[4′-(1-Piperidin-1-ylethyl)-biphenyl-4-yl]-pyrimidine,
2-[4′-(1-Methylpiperidin-2-yl)-biphenyl-4-yl]-pyrimidine,
5-[4′-(1-Methylpiperidin-2-yl)-biphenyl-4-yl]-pyrimidine,
3-[4′-(1-Methylpiperidin-2-yl)-biphenyl-4-yl]-pyridine,
2,6-Dimethyl-3-[4′-(1-methylpiperidin-2-yl)-biphenyl-4-yl]-pyridine,
2-Fluoro-5-[4′-(1-methylpiperidin-2-yl)-biphenyl-4-yl]-pyrimidine,
4-[4′-(1-Methylpiperidin-2-yl)-biphenyl-4-yl]-pyridine,
1-Methyl-2-(2′-methyl-4′-pyrrol-1-ylbiphenyl-4-yl)-piperidine,
1-Methyl-2-[4′-(2-methylimidazol-1-yl)-biphenyl-4-yl]-piperidine,
4-[4′-(1-Methylpiperidin-2-yl)-biphenyl-4-yl]-1H-pyrido[1,2-c]pyrimidine,
4-[4′-(1-Methylpiperidin-2-yl)-biphenyl-4-yl]-isoquinoline,
5-{4′-[1-(2,6-Dimethylpiperidin-1-yl)-ethyl]-biphenyl-4-yl}-2,4-dimethylpyrimidine,
2-Methyl-5-[3′-methyl-4′-(1-piperidin-1-ylethyl)-biphenyl-4-yl]-pyrimidine and
[1-(2′,6′-Dimethyl-4′-thiazol-2-ylbiphenyl-4-yl)-ethyl]-dimethylamine.

### DETAILED DESCRIPTION OF THE INVENTION

The compound of formula (I) according to the invention may be prepared by the general procedure shown in Scheme 1.

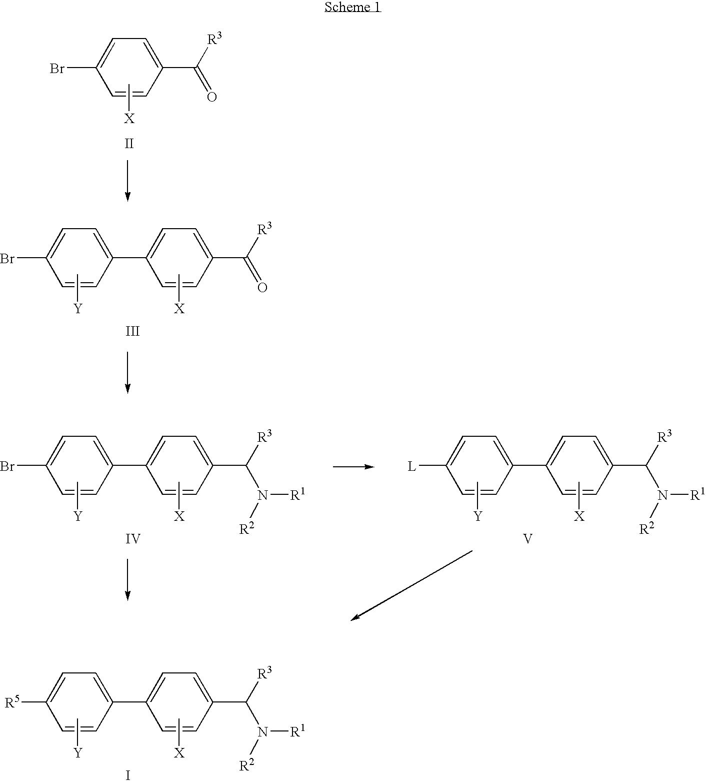

In Scheme 1, compounds of the formula (I) are prepared as follows.

A ketone (R³≠H, as previously defined) of the general formula II may be reacted with a compound of the general formula IX: 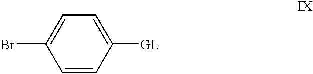
wherein the group GL is defined as a leaving group, to provide an aldehyde or ketone of the general formula III. One such variation on this procedure is the Suzuki reaction, which has been described in numerous publications in the scientific literature, including Stanforth, S. P., “Catalytic Cross-coupling Reactions in Biaryl Synthesis.” Tetrahedron, 1998, 54:263-303; Watanabe, T. et al “Synthesis of Sterically Hindered Biaryls via the Palladium-catalyzed Cross-coupling Reaction of Arylboronic Acids or Their Esters with Haloarenes.” Synlett, 1992, 3:207-210; Ali, N. M. et al “Palladium-catalyzed Cross-coupling Reactions of Arylboronic Acids with π-Deficient Heteroaryl Chlorides.” Tetrahedron, 48(37):8117-8126; Saito, S. et al “Synthesis of Biarlys via a Nickel(0)-catalyzed Cross-coupling Reaction of Chloroarenes with Arylboronic Acids.” Journal of Organic Chemistry, 1997, 62(23):8024-8030; Indolese, A. F. “Suzuki-type Coupling of Chloroarenes with Arylboronic Acids Catalyzed by Nickel Complexes.” Tetrahedron Letters, 1997, 38(20):3513-3516; Zhang, H. et al, “Base and Cation Effects on the Suzuki Cross-coupling of Bulky Arylboronic Acid with Halopyridines. Synthesis of Pyridylphenols.” Journal of Organic Chemistr, 1988, 63(20):6886-6890; Wustrow, D. J. and Wise, L. D. “Coupling of Arylboronic Acid with a Partially Reduced Pyridine Derivative.” Synthesis, 1991, 11:993-995; and many others. Using such conditions, reaction of a 4-bromophenyl ketone with 4-bromophenylboronic acid, in the presence of a metal catalyst and a base will generate a biphenylyl ketone of the formula III. The ketones of formula II used in this process can be obtained from commercial sources or readily prepared by methods known to one skilled in the art. The boronic acids used in this process can also be obtained commercially, or prepared, as described in the chemical literature. The base used in the reaction can be selected from, but is not limited to, cesium carbonate, sodium carbonate, potassium carbonate, sodium bicarbonate, potassium bicarbonate, sodium hydroxide, potassium hydroxide and the like, preferably sodium carbonate. The catalyst can also be selected from one of the many palladium catalysts that have been described in the literature, several of which are commercially available, including but not limited to Pd₂(dba)₃ with triphenylphoshine or tri-tert-butylphosphine, tetrakis(triphenylphoshine)palladium(0), dichloro-bis(triphenylphoshine) palladium(0), and the like. The choices for solvent used in this reaction step include aqueous methanol or aqueous ethanol, or ethers like 1,4-dioxane, THF and dimethoxyethane (DME). The reaction is most effective when run at room temperature, but at least in the range of about 0-100° C. and preferentially at atmospheric pressure.

Intermediates of general formula III may then be reacted with primary or secondary amines of general formula HNR¹R² (X), where R¹ and R² are as defined in the specification. This can be accomplished, for example, using a procedure referred to as reductive amination which is a method well known to those skilled in the art. This method may be conducted in a single, concerted process (e.g., see A. F. Abdel-Magid, C. A. Maryanoff and K. G. Carson in Tetrahedron Letters, 1990, 39:5595-5598). In such conversions, the carbonyl compound of formula III and the appropriate amine of formula X are combined in a reaction inert solvent and treated with reagents like sodium cyanoborohydride or sodium triacetoxyborohydride. Suitable solvents include, among others, tetrahydrofuran (THF) and 1,2-dichloroethane (DCE) and the reactions may be conducted with or without the addition of an organic acid (e.g., acetic acid).

Alternatively, the conversion of compounds of formula III to compounds of formula IV can be completed using two or more individual steps, involving the initial formation of an imine intermediate such as Xi, followed by reduction of the C═N double bond to generate IV.

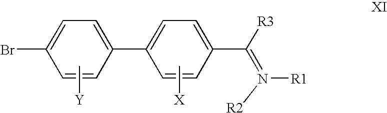

For example, the intermediate of formula III and the amine X of formula HNR¹R² can be combined in the presence of a dehydrating reagent in a reaction neutral solvent like benzene, toluene, methanol or ethanol and stirred for a prescribed amount of time until the reaction is judged to be completed. Such dehydrating reagents include, for example, p-toluenesulfonic acid, titanium(IV)chloride, titanium(IV) isopropoxide or molecular sieves. The reaction can be conducted within the range of about 0° C. to about the boiling point of the solvent employed and at pressures of about one to about three atmospheres. The intermediate imine XI so obtained can then be reduced with a variety of reagents and under a variety of conditions familiar to one skilled in the art, including the use of hydrogen gas in the presence of a catalyst like palladium on carbon (Pd/C) or platinum on carbon (Pt/C), as well as with sodium borohydride, sodium (triacetoxy)borohydride, sodium cyanoborohydride and the like. The use of hydrogen as the reducing agent is often conducted in a reaction inert solvent such as methanol, ethanol, THF, 1,4-dioxane and similar solvents at a pressure of about one atmosphere to a pressure of about 5 atmospheres of hydrogen and typically at a temperature from about room temperature to a temperature that is below the boiling point of the solvent employed. When using the hydride reagents, the choice of solvent can be made from, but not limited to, methanol, ethanol, isopropanol, 1,4-dioxane, THF and the like. The reaction can generally be carried out at atmospheric pressure and at temperatures ranging from about −40° C. to about the boiling temperature of the solvent employed, typically at 0-40° C. and most preferably at room temperature.

Finally, the compounds of formula I can be prepared by reacting the intermediate compounds of general formula IV with a compound of general formula R⁵-GL²(XII), where R⁵ is as defined in the specification section of this application and GL² is a leaving group. For example, when GL² is —B(OH)₂, the compounds of formula IV and formula XII can be reacted under the Suzuki coupling conditions described above (for the conversion of compounds of general formula II to those of general formula II) to prepare the compounds of general formula I. Alternatively, the intermediate of formula IV can be converted into an intermediate of formula V, wherein the group L is a suitable leaving group that can then be reacted with a compound of general formula R⁵-GL³ (XIII). This route of synthesis may be preferable when preparing a variety of analogs wherein the availability of intermediates XII is not as good as for intermediates of formula XIII, for example in the synthesis of compounds using the Suzuki coupling reaction where R⁵ bromides and iodides are more accessible than R⁵ boronic acids.

The compound of formula (I) wherein NR²R³ is a heterocyclic ring system of 4-8 atoms, according to the invention, may be prepared by the general procedure shown in Scheme 2.

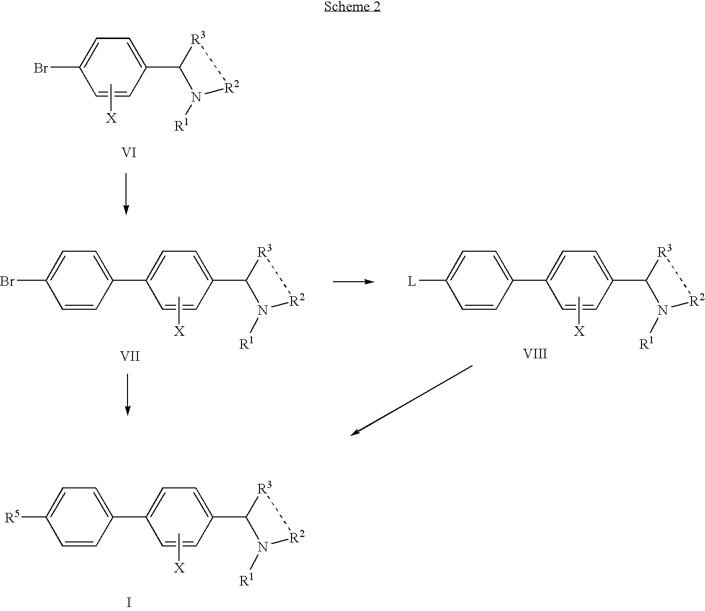

Thus, compounds of intermediate formula VI, which are either known or readily prepared using methods and procedures described in the scientific literature, are reacted under Suzuki coupling conditions as previously described for the conversion of II to III to generate the intermediate bromides of general formula VII. Such intermediates so obtained can then be converted directly into the desired compounds of general formula I using Suzuki conditions as described above. Alternatively, the intermediates of general formula VII can be first converted to the intermediate of formula VIII in the manner described above for the conversion of compounds IV to V, and then reacted with a compound of general formula R⁵GL³ to give the desired product of general formula 1.

In the examples below the following terms are intended to have the following, general meaning:
bs: broad singlet
d.e.: diatomaceous earth, filter agent
DMF: dimethylormamide
LRMS: low resolution mass spectrometry
calcd; calculated
d; doublet (spectral)
EtOAc: ethyl acetate
J: coupling constant (in NMR)
LAH: lithium aluminum hydride
m: multiplet (in NMR)
Min: minute(s)
m/z: mass to charge ratio (in mass spectrometry)
obsd: observed
Rf: retention factor (in chromatography)
Rt: retention time (in chromatography)
rt: room temperature
s: singlet (NMR), second(s)
t: triplet
TFA: trifluoroacetic acid
TFAA: trifluoroacetic anhydride
THF: tetrahydrofuran
tlc: thin layer chromatography

Solvents were purchased and used without purification. Yields were calculated for material judged homogenous by thin layer chromatography and NMR. Thin layer chromatography was performed on Merck Kieselgel 60 F 254 plates eluting with the solvents indicated, visualized by a 254 nm UV lamp, and stained with either an aqueous KMnO₄ solution or an ethanolic solution of 12-molybdophosphoric acid. Flash column chromatography was performed with using either pre-packed Biotage® or ISCO® columns using the size indicated. Nuclear magnetic resonance (NMR) spectra were acquired on a Unity 400 or 500 at 400 MHz or 500 MHz for ¹H, respectively, and 100 MHz or 125 MHz for ¹³C NMR, respectively. Chemical shifts for proton ¹H NMR spectra are reported in parts per million relative to the singlet of CDCl₃ at 7.24 ppm. Chemical shifts for ¹³C NMR spectra are reported in parts per million downfield relative to the centerline of the triplet of CDCl₃ at 77.0 ppm. Mass spectra analyses were performed on a APCI Gilson 215, micromass ZMD (50% Acetonitrile/50% water) spectrometer.

Reactions under microwave conditions were done using 2-5 mL round bottom vials, fitted with septa. The vials containing the reactants were inserted into the reaction chamber of a EMRYS™ Creator microwave apparatus (maximum power of 300 W) from Personal Chemistry Inc., 25 Birch St., Bldg C, Suite 304, Milford, Mass. 01757 and heated to the appropriate temperature for a the prescribed period of time. HPLC was performed according to the following methods:

Method A: Preparative conditions (Waters 600 & Waters 2767 Sample Manager); Column: Waters Symmetry C₁₈, 5 μm, 30×150 mm steel column, part # WAT248000, serial # M12921A01; solvent A—0.1% Trifluoroacetic acid/water; solvent B—Acetonitrile; volume of injection: 850 μL; time 0.0, 100% solvent A, 0% solvent B, flow 20; time 2.0, 100% solvent A, 0% solvent B, flow 20; time 12.0, 0% solvent A, 100% solvent B, flow 20; time 15.0, 0% solvent A, 100% solvent B, flow 20; time 15.1, 100% solvent A, 0% solvent B, flow 20; time 20.0, 100% solvent A, 0% solvent B, flow 20.

Mass spectral (micromassZO) conditions; Capillary(kV): 3.0; Cone (V): 20; Extractor (V): 3.0; RF Lens (V): 0.5; Source temp. (° C.): 120; Desolvation temp. (° C.): 360; Desolvation gas flow (L/hr): 450; Cone gas flow (L/hr): 150; LM Resolution: 15; HM Resolution: 15; Ion Energy: 0.2; Multiplier: 550.

Splitter; Acurate by LC Packings, 1/10,000; Upchurch needle valve setting: 14; Make up pump (Waters 515) Flow (ml/min.): 1. PDA (Waters 996) Settings; Start/End wavelength (nm): 200/600; Resolution: 1.2; Sample Rate: 1; Channels: TIC, 254 nm and 220 nm.

Method B: Preparative conditions (Waters 600 & Waters 2767 Sample Manager); Column: Waters Xterra PrepMS C₁₈ column, 5 μm, 30×150 mm steel column, part # 186001120, serial # T22881T 09; solvent A—0.1% Trifluoroacetic acid/water; solvent B—Acetonitrile; volume of injection: 1050 μL; time 0.0, 100% solvent A, 0% solvent B, flow 20; time 2.0, 100% solvent A, 0% solvent B, flow 20; time 12.0, 0% solvent A, 100% solvent B, flow 20; time 14.0, 0% solvent A, 100% solvent B, flow 20; time 14.1, 100% solvent A, 0% solvent B, flow 20; time 19.1, 100% solvent A, 0% solvent B, flow 20.

Mass spectral (micromassZO) conditions; Capillary(kV): 3.0; Cone (V): 20; Extractor (V): 3.0; RF Lens (V): 0.5; Source temp. (° C.): 120; Desolvation temp. (° C.): 360; Desolvation gas flow (L/hr): 450; Cone gas flow (L/hr): 150; LM Resolution: 15; HM Resolution: 15; Ion Energy: 0.2; Multiplier: 550.

Splitter; Acurate by LC Packings, 1/10,000; Upchurch needle valve setting: 14; Make up pump (Waters 515) Flow (ml/min.): 1. PDA (Waters 996) Settings; Start/End wavelength (nm): 200/600; Resolution: 1.2; Sample Rate: 1; Channels: TIC, 254 nm and 220 nm.

Method C: Preparative conditions (Waters 600 & Waters 2767 Sample Manager); Column: Waters Symmetry C₁₈, 5 μm, 30×150 mm steel column, part # WAT248000, serial # M12921A01; solvent A —0.1% Trifluoroacetic acid/water; solvent B—Acetonitrile; volume of injection: 850 μL; time 0.0, 90% solvent A, 10% solvent B, flow 20; time 10.0, 0% solvent A, 100% solvent B, flow 20; time 12.0, 0% solvent A, 100% solvent B, flow 20.

Mass spectral (micromassZO) conditions; Capillary(kV): 3.0; Cone (V): 20; Extractor (V): 3.0; RF Lens (V): 0.5; Source temp. (° C.): 120; Desolvation temp. (° C.): 360; Desolvation gas flow (L/hr): 450; Cone gas flow (L/hr): 150; LM Resolution: 15; HM Resolution: 15; Ion Energy: 0.2; Multiplier: 550. Splitter; Acurate by LC Packings, 1/10,000; Upchurch needle valve setting: 14; Make up pump (Waters 515) Flow (ml/min.): 1. PDA (Waters 996) Settings; Start/End wavelength (nm): 200/600; Resolution: 1.2; Sample Rate: 1; Channels: TIC, 254 nm and 220 nm.

The following intermediates may be prepared by the procedures described above:

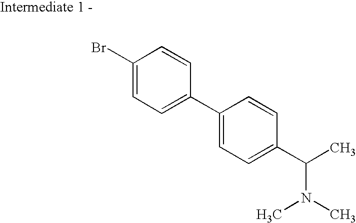

### [1-(4′-Bromobiphenyl-4-yl)-ethyl]-dimethylamine

A stirred solution of 4-(4-bromophenyl)-acetophenone (6.6 g, 24 mmol, Aldrich Chemical Co.) in 240 mL of a 2.0 M solution of dimethylamine in methanol at 0° C. (ice/water bath) was treated dropwise with titanium (IV) isopropoxide (12.0 mL, 480 mmol). After the addition was complete the reaction was stirred at room temperature for 72 hours. Solid sodium borohydride (1.86 g, 24.0 mmol) was added portionwise over thirty minutes, with stirring continued for another 4 hours. The solvent was then removed in vacuo and the residue partitioned between 100 mL water and 75 mL methylene chloride. The emulsion was treated with 1N HCl to a pH of 3.0-3.5, stirred another 2 hours, then readjusted to pH 9.0 with 2N NaOH. After another hour of stirring, the mixture was extracted with additional methylene chloride. These extracts were washed with water, dried over MgSO₄, filtered and concentrated to a white solid. Flash chromatography using a gradient of 0-4% methanol in methylene chloride gave, after removal of the solvent, 0.80 g of off-white solid.

Mass spectrum (m/z) calcd for C₁₆H₁₈BrN: 304.23; obsd. 307, 305 (M+1), 290, 288.

¹H-nmr (CDCl₃, 400 MHz) δ 1.39 (d, 3H), 2.20 (s, 6H), 3.28 (q, 1H), 7.24 (s, 1H), 7.37-7.55 (m, 7H).

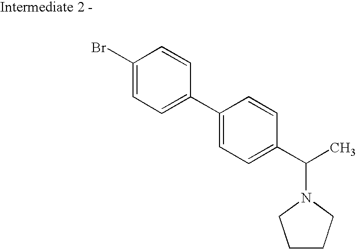

### 1-[1-(4′-Bromobiphenyl-4-yl)-ethyl]-pyrrolidine

This was prepared in the same manner as intermediate 1, replacing the dimethylamine with pyrrolidine to give a pale yellow solid.

Mass spectrum (m/z) calcd for C₁₈H₂₀BrN: 330.27; obsd: 332, 330 (100%, M+1).

¹H-nmr (CDCl₃, 400 MHz) δ 1.39 (d, 3H), 1.75 (m, 4H), 2.38 (m, 2H), 2.54 (m, 2H), 3.19 (q, 1H), 7.23 (s, 1H), 7.36-7.53 (m, 7H).

A 6.5 g sample of the racemic 1-[1-(4′-bromobiphenyl-4-yl)-ethyl]-pyrrolidine was separated into the respective enantiomers using flash chromatography.

The first enantiomer to elute from the column was obtained as a white fluffy solid, 2.94 g.
[α]²⁵_{D}=+36.8° (c=1, CH₃OH).

This compound was assigned the (R) configuration based upon X-ray crystallography data.

The second, more polar, S-enantiomer was isolated as a light yellow crystalline solid, 2.82 g.
[α]²⁵_{D}=−36.8° (c=1, CH₃OH).

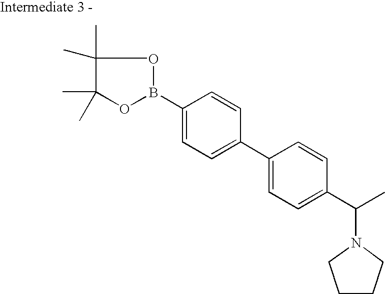

### (±)1-{1-[4′-(4,4,5,5-Tetramethyl-[1,3,2]dioxaborolan-2-yl)-biphenyl-4-yl]-ethyl}-pyrrolidine

This was prepared according to the method of Murata et al, Journal of Organic Chemistry, 1997, 62:6458-6459. A mixture of racemic 1-[1-(4′-bromobiphenyl-4-yl)-ethyl]-pyrrolidine (0.330 g, 1.0 mmol, intermediate 2), triethylamine (0.42 mL, 3.0 mmol), 4,4,5,5-tetramethyl-1,3,2-dioxaborolane (1.5 mL, 1.5 mmol, 1 M in THF from Aldrich Chemical Company) and 1,1-bis-(diphenylphosphino)ferrocene palladium (II) chloride (22 mg, 0.03 mmol) in 4.0 mL of acetonitrile was heated to 80° C. until the reaction was determined to be complete by tlc. It was used without further purification to prepare compounds listed below.

Mass spectrum (m/z) calcd for C₂₄H₃₂BNO₂: 377.34; obsd: 379, 378 (M+1, 100%), 377, 307.

¹H-nmr (CDCl₃, 400 MHz) δ 1.20 (m, 2H), 1.33 (m, 3H), 1.64 (bs, 1H), 1.88 (bs, 4H), 2.01 (bs, 1H), 2.17 (bs, 1H), 2.33 (bs, 1H), 2.68 (bs, 1H), 2.88 (bs, 1H), 3.31 (bs, 1H), 4.00 (bs, 2H), 7.23 (s, 1H), 7.53-7.71 (m, 6H), 7.86 (d, 1H).

The (S)- and (R)-enantiomers were prepared in a similar manner from the corresponding (S)- and (R)-bromides described in intermediate 2.

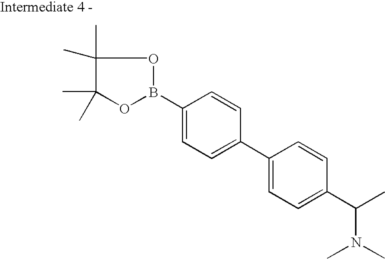

### (±)-Dimethyl-{1-[4′-(4,4,5,5-tetramethyl-[1,3,2]dioxaborolan-2-yl)-biphenyl-4-yl]-ethyl}-amine

This was prepared in the same manner as intermediate 3 above, beginning with 120 mg of [1-(4′-bromobiphenyl-4-yl)-ethyl]-dimethylamine (intermediate 1), to provide a dark amber gum.

Mass spectrum (m/z) calcd for C₂₂H₃₀BNO₂: 351.30; obsd: 352 (M+1, 100%), 307, 267.

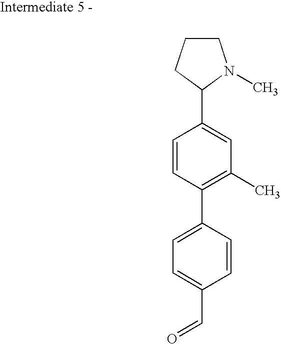

### 2′-Methyl-4′-(1-methylpyrrolidin-2-yl)-biphenyl-4-carboxaldehyde

In a 5 mL microwave tube, a mixture of 2-(4-bromo-3-methylphenyl)-1-methylpyrrolidine (159 mg, 0.63 mmol), 4-formylphenylboronic acid (188 mg, 1.25 mmol, Aldrich Chemical Company), sodium carbonate (331 mg, 3.15 mmol) and tetrakis(triphenylphosphine)palladium(0) in 4.0 mL of ethanol containing 1.0 mL water were heated to 150° C. for 10 min. After cooling to room temperature, the mixture was diluted with water and methylene chloride, made basic with saturated aqueous sodium carbonate and filtered through a pad of d.e. The organic layer was combined with two additional CH₂Cl₂ extractions of the aqueous layer and washed with saturated aqueous NaCl. Removal of the solvent in vacuo gave a light brown oil, 163 mg. Chromatography on silica gel, eluting with chloroform gave 104 mg of tan oil.

Mass spectrum (m/z) calcd for C₁₈H₁₉NO: 265.36; obsd: 266 (M+1, 100%),

¹H-nmr (CDCl₃, 400 MHz) δ 1.81 (m, 1H), 1.96 (m, 1H), 2.16 (m, 1H), 2.20 (s, 3H), 2.22 (m, 1H), 2.25 (s, 3H), 3.04 (t, 1H), 3.23 (t, 1H), 7.25 (m, 3H), 7.51 (d, 2H), 7.89 (d, 2H), 10.03 (s, 1H).

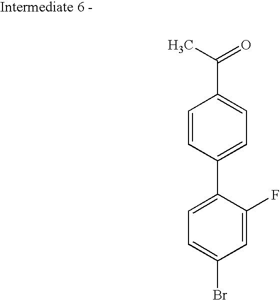

### 1-(4′-Bromo-2′-fluorobiphenyl-4-yl)-ethanone

To a mixture of aluminum chloride (2.93 g, 22 mmol) in 20 mL of 1,1,2,2-tetrachloroethane, cooled in an ice water bath, was added 4-bromo-2-fluorobiphenyl (2.51 g, 10 mmol). Acetyl chloride (0.942 mg, 12 mmol) was added slowly via syringe and the mixture stirred for 20 hours, allowing it to warm gradually to room temperature. The mixture was then poured over 20 mL of ice cold 6 N HCl, stirred 1 hr and extracted with chloroform. The organic extracts were washed with water, dilute aqueous NaHCO₃ and water. After drying with MgSO₄, the solvent was removed in vacuo to give an amber oil.

Mass spectrum (m/z) calcd for C₁₄H₁₀BrFO: 292; obsd 292 (M+), 294 (M+2)

¹H-nmr (CDCl₃, 400 MHz) δ 2.62 (s, 3H), 7.33 (m, 2H), 7.36 (s, 1H), 7.59 (dd, 2H).

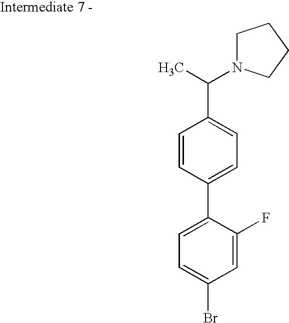

### 1-[1-(4′-Bromo-2′-fluorobiphenyl-4-yl)-ethyl]-pyrrolidine

A solution of 1-(4′-bromo-2′-fluorobiphenyl-4-yl)-ethanone, from the preceding step, in 100 mL of methanol at rt was treated with pyrrolidine (1.75 g, 24.5 mmol) followed by titanium isopropoxide (6.98 g, 7.3 mL, 24.5 mmol, Aldrich Chemical Co.) over a 5-min period via syringe. After stirring at rt overnight, the reaction was cooled with an ice bath and sodium borohydride (0.696 g, 18.4 mmol) was added in small portions (foaming) and the mixture was allowed to stir at rt for another 24 hr. The mixture was then quenched with 6N HCl and stirred for another 30 min, at which time it was diluted with water and EtOAc, filtered to remove some insolubles, and the organic layer was combined with additional EtOAc extractions of the aqueous layer. The combined organic extracts were washed with water and saturated aqueous NaCl, dried with MgSO₄ and concentrated to a brown tarry residue, 2.35 g. This residue was flash chromatographed on silica gel, eluting with 100% EtOAc followed by 95% EtOAc with 5% CH₃OH. The polar fraction containing the purified product was concentrated to a light brown oil, 0.123 g.

Mass spectrum (m/z) calcd for C₁₈H₁₇BrFN: 348.25; obsd 348 (M+), 350 (M+2)

¹H-nmr (CDCl₃, 400 MHz) δ 1.40 (d, 3H), 1.74 (m, 4H), 2.38 (bm, 2H), 2.54 (bt, 2H), 3.21 (q, 1H), 7.30 (m, 3H), 7.41 (m, 4H).

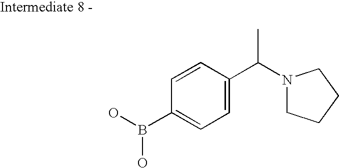

### 4-(1-(Pyrrolidin-1-yl)ethyl)-phenylboronic acid

A mixture of 4-acetylphenylboronic acid (61.4 g, 0.1 mol) in 400 mL methanol containing activated 4 Å molecular sieves was stirred under N2 while pyrrolidine (84 mL, 1.0 mol) was added via syringe—slight exotherm. After stirring at rt overnight, the reactants were filtered and the filtrate was hydrogenated in the presence of 7 g of 10% Pd-on-carbon at an initial pressure of 45 psi for 3 hours. The reaction was filtered through diatomaceous earth (d.e.) and concentrated in vacuo to an amorphous yellow solid, 20 g.

Mass spectrum (m/z) calcd for C₁₂H₁₈BNO₂: 220; obsd 220.2 (M+).

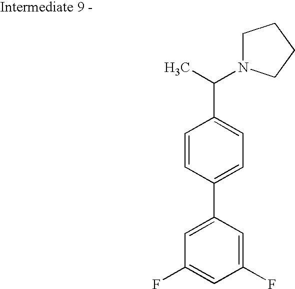

### 1-[1-(3′,5′-Difluoro-biphenyl-4-yl)-ethyl]-pyrrolidine

A mixture of 4-(1-(pyrrolidin-1-yl)ethyl)-phenylboronic acid (440 mg, 2.0 mmol), 1-bromo-3,5-difluorobenzene (580 mg, 3.0 mmol), sodium carbonate (848 mg, 8.0 mmol) and tetrakis(triphenylphosphine)palladium(0) (64 mg, 0.04 mmol) was dissolved in 15 mL ethanol containing 3.0 mL H₂O, degassed and reacted in a microwave apparatus at 150° C. for 5.0 min. After cooling to rt and filtering through a pad of d.e., the filtrate was diluted with methylene chloride, washed with water and saturated NaCl, then dried over Na₂SO₄ and filtered. Removal of the solvent in vacuo gave a viscous light brown syrup. Flash chromatography on silica gel, eluting with a gradient (100% CH₂Cl₂ to 5% CH₃OH:95% CH₂Cl₂) gave a light brown gum, 134 mg.

Mass spectrum (m/z) calcd for C₁₈H₁₉F₂N, 287.35; obsd: 288 (M+).

¹H-nmr (CDCl₃, 400 MHz) δ 1.54 (d, 3H), 1.86 (bs, 4H), 2.59 (bs, 2H), 2.76 (bs, 2H), 3.40 (bs, 1H), 6.76 (m, 1H), 7.07 (m, 2H), 7.50 (m, 4H).

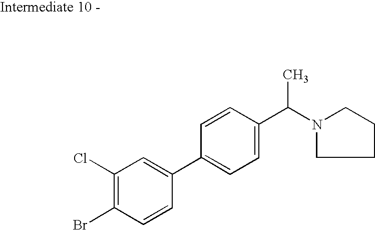

### 1-[1-(4′-Bromo-3′-chloro-biphenyl-4-yl)-ethyl]-pyrrolidine

Prepared in the same manner as intermediate 9, using 1,4-dibromo-2-chlorobenzenzene (810 mg, 3.0 mmol) to give the title product as a viscous yellow syrup, 168 mg.

Mass spectrum (m/z) calcd for C₁₈H₁₉BrClN: 365; obsd: 366 (M+).

¹H-nmr (CDCl₃, 400 MHz) δ 1.40 (d, 3H), 1.76 (d, 3H), 2.40 (bs, 2H), 2.55 (bs, 2H), 3.21 (m, 1H), 7.19-7.66 (m, 7H).

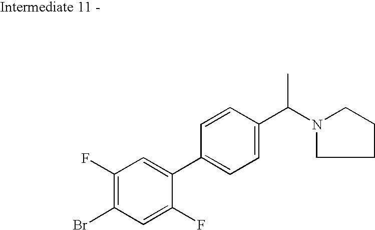

### 1-[1-(4′-Bromo-2′,5′-difluoro-biphenyl-4-yl)-ethyl]-pyrrolidine

Prepared in the same manner as intermediate 9, using 1,4-dibromo-2,5-difluorobenzene (816 mg, 3.0 mmol) to give the title product as pale yellow glue, 188 mg.

Mass spectrum (m/z) calcd for C₁₈H₁₈BrF₂N, 366.25; obsd: 366 (M+), 368 (M+2).

¹H-nmr (CDCl₃, 400 MHz) δ 1.41 (d, 3H), 1.59 (bs, 2H), 1.76 (bs, 4H), 2.39 (bs, 2H), 2.55 (bs, 2H), 3.20 (q, 1H), 7.18-7.66 (m, 6H).

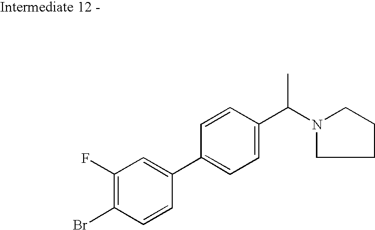

### 1-[1-(4′-Bromo-3′-fluoro-biphenyl-4-yl)-ethyl]-pyrrolidine

Prepared in the same manner as intermediate 9 above, using 1,4-dibromo-2-fluorobenzene (760 mg, 3.0 mmol) to give the title product as a viscous amber colored oil, 250 mg.

Mass spectrum (m/z) calcd for C₁₈H₁₉BrFN: 348.26; obsd: 348 (M+), 350 (M+2).

¹H-nmr (CDCl₃, 400 MHz) δ 1.42 (d, 3H), 1.78 (bs, 4H), 2.40 (bs, 2H), 2.57 (BS, 2H), 3.23 (q, 1H), 7.24-7.60 (m, 7H).

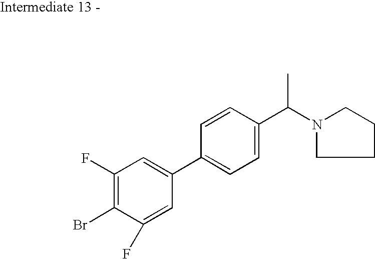

### 1-[1-(4′-Bromo-3′,5′-difluoro-biphenyl-4-yl)-ethyl]-pyrrolidine

1-[1-(3′,5′-Difluoro-biphenyl-4-yl)-ethyl]-pyrrolidine (134 mg, 0.47 mmol)) the title compound of intermediate 9, was dissolved in 10 mL of THF, cooled to −70° C. and treated with 0.4 mL (1.0 mmol) of a 2.5 M n-butyl lithium in THF solution. After stirring a further 25 min at −70° C., bromine (83 mg, 0.52 mmol, dissolved in 1 mL THF) was added. The reaction was then allowed to warm to rt. Removal of the solvent in vacuo gave a gummy rsidue which was redissolved in methylene chloride, washed with water and saturated NaCl, then dried over Na₂SO4. Removal of the solvent and flash chromatography on silica gel using a CH₂Cl₂ to 3% CH₃OH:97% CH₂Cl₂ gradient in 0.5% increments gave purified product as a pale yellow oil, 44 mg.

Mass spectrum (m/z) calcd for C₁₈H₁₈BrF₂N, 366.25; obsd: 366 (M+), 368 (M+2).

1H-nmr (CDCl₃, 400 MHz) δ 1.40 (d, 3H), 1.75 (m, 4H), 2.38 (bs, 2H), 2.55 (bs, 2H), 3.22 (q, 1H), 7.07-7.48 (m, 6H).

The following compounds may be prepared by the procedures below:

### EXAMPLE 1

### General Procedure A

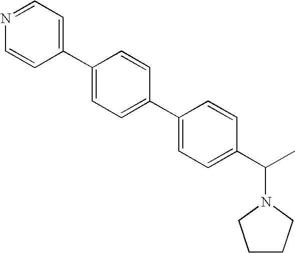

### (R)-(±)-4-[4′-(1-Pyrrolidin-1-ylethyl)-biphenyl-4-yl]-pyridine

A mixture of (R)-(±)-1-[1-(4′-bromobiphenyl-4-yl)-ethyl]-pyrrolidine (165 mg, 0.5 mmol), pyridine-4-boronic acid (74 mg, 0.06 mol) sodium carbonate (212 mg, 2.0 mmol) and tetrakis(triphenylphosphine)-palladium(0) in 3.8 mL ethanol containing 0.8 mL water was added to a 5 mL microwave tube and degassed. The tube was sealed, placed in the microwave apparatus and the contents were irradiated at 150° C. for 300 sec. After cooling to room temperature, the crude product was isolated by extraction into methylene chloride. The extracts were washed with water, dried with MgSO₄ and concentrated in vacuo to produce a yellow viscous oil. The crude product was flash chromatographed using a gradient system of 04% methanol in methylene chloride and the fractions containing pure product were concentrated in vacuo to a white solid, 137 mg. This material was then converted to the hydrochloride salt (134 mg) by dissolving the free base in a minimal amount of ethyl acetate, adding an excess of 1.0 M HCl in diethyl ether (Aldrich Chemical Company) and stirring the resulting white solid at room temperature for 0.5-1.0 hr before filtering, washing with Et₂O and drying under vacuum.

Mass spectrum (m/z) calcd for C₂₃H₂₄N₂: 328.42; obsd: 330, 329 (M+1, 100%), 258.

¹H-nmr (CDCl₃, 400 MHz) δ 1.47 (d, 3H), 1.80 (bs, 4H), 2.55 (bs, 2H), 2.63 (bs, 2H), 3.31 (s, 1H), 7.45 (m, 2H), 7.55 (m, 4H), 7.71 (m, 4H), 8.65 (d, 2H).

### EXAMPLE 2

### General Procedure A

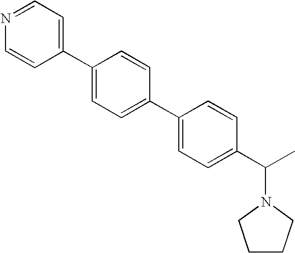

### (S)-(−)-4-[4′-(1-Pyrrolidin-1-ylethyl)-biphenyl-4-yl]-pyridine

Prepared as described in Example 1, as a white solid and converted to the hydrochloride salt, which was isolated as a white powder.

Mass spectrum (m/z) calcd for C₂₃H₂₄N₂: 328.42; obsd: 330, 329 (M+1, 100%), 258.

¹H-nmr (CDCl₃, 400 MHz)—identical to that listed for the (R)-enantiomer in example 1.

### EXAMPLE 3

### General Procedure A

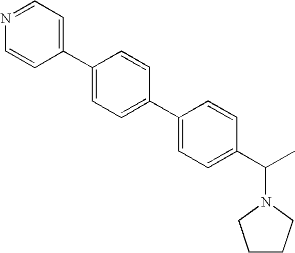

### (±)-4-[4′-(1-Pyrrolidin-1-ylethyl)-biphenyl-4-yl]-pyridine

The racemic mixture of compounds described in Examples 1 and 2 was also prepared starting with racemic bromide (intermediate 2).

Mass spectrum (m/z) calcd for C₂₃H₂₄N₂: 328.42; obsd: 330, 329 (M+1, 100%), 258.

### EXAMPLE 4

### General Procedure A

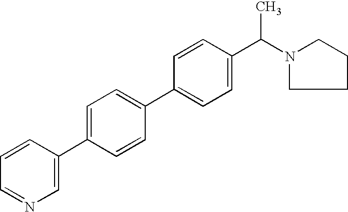

### (R)-(3-[4′-(1-Pyrrolidin-1-ylethyl)-biphenyl-4-yl]-Pyridine

This compound was prepared in the manner described for Example 1. Thus, (R)-(±)-1-[1-(4′-bromobiphenyl-4-yl)-ethyl]-pyrrolidine (165 mg, 0.5 mmol) and diethyl (3-pyridyl)borane (88 mg, 0.6 mmol) gave 113 mg of the free base as a white solid. This was converted to the hydrochloride salt as described in Example 1.

Mass spectrum (m/z) calcd for C₂₃H₂₄N₂: 328.42; obsd: 330, 329 (M+1), 279, 258.

¹H-nmr (CDCl₃, 400 MHz)-δ 1.42 (d, 3H), 1.77 (m, 4H), 2.40 (m, 2H), 2.56 (m, 2H), 7.24 (s, 1H), 7.34-7.42 (m, 4H), 7.55-7.70 (m, 5H), 7.90 (m, 1H), 8.57 (m, 1H), 8.88 (d, 1H).

### EXAMPLE 5

### General Procedure A

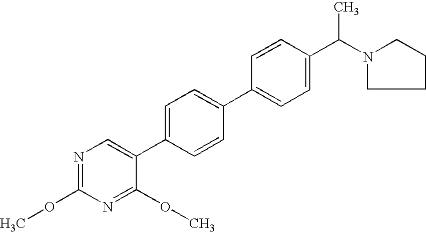

### (R)-2,4-Dimethoxy-5-[4′-(1-pyrrolidin-1-ylethyl)-biphenyl-4-yl]-pyrimidine

(R)-(±)-1-[1-(4′-bromobiphenyl-4-yl)-ethyl]-pyrrolidine (165 mg, 0.5 mmol) and 2,4-dimethoxypyrimidine-5-boronic acid (138 mg, 0.75 mmol) in 3.8 mL ethanol containing 0.8 mL water were combined with sodium carbonate (212 mg) and tetrakis(triphenylphosphine)palladium(0) in a 5 mL microwave tube. The reactants were heated in a microwave apparatus for 300 min at 150° C., cooled to room temperature and the crude product was purified as described previously. The free base was isolated as a clear oil that was converted to the hydrochloride salt as a white solid, 73 mg.

Mass spectrum (m/z) calcd for C₂₄H₂₇N₃O₂: 389.50; obsd: 391, 390 (M+1, 100%), 319, 279.

¹H-nmr (CDCl₃, 400 MHz)-δ 1.43 (d, 3H), 1.77 (bs, 4H), 2.42 (bs, 2H), 2.63 (bs, 2H), 3.22 (q, 1H), 4.03 (s, 6H), 7.24 (s, 1H), 7.40 (m, 2H), 7.55 (m, 3H), 7.62 (m, 2H), 8.30 (s, 1H).

### EXAMPLE 6

### General Procedure A

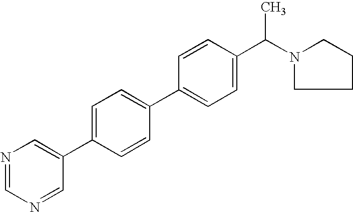

### (±)-5-[4′-(1-Pyrrolidin-1-ylethyl)-biphenyl-4-yl]-pyrimidine

This compound was prepared from the racemic bromide (intermediate 2). Thus, (±)-1-[1-(4′-bromobiphenyl-4-yl)-ethyl]-pyrrolidine (83 mg, 0.25 mmol) and pyrimidine-5-boronic acid (47 mg, 0.38 mmol) were reacted to give the crude product, isolated as a light brown oil. The oil was converted to the hydrochloride salt in the manner previously described.

Mass spectrum (m/z) calcd for C₂₂H₂₃N₃: 329.44; obsd: 331, 330 (M+1, 100%), 259.

¹H-nmr (CDCl₃, 400 MHz)-δ 1.44 (d, 3H), 1.79 (m, 4H), 2.44 (bs, 2H), 2.60 (bs, 2H), 3.27 (q, 1H), 7.24 (s, 1H), 7.45 (m, 2H), 7.64 (m, 2H), 7.72 (m, 2H), 8.98 (s, 2H), 9.19 (s, 1H).

### EXAMPLE 7

### General Procedure A

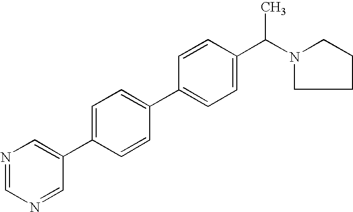

### (R)-(±)-5-[4′-(1-Pyrrolidin-1-ylethyl)-biphenyl-4-yl]-pyrimidine

This enantiomer was prepared according to the procedure of Example 6, replacing racemic (±)-1-[1-(4′-bromobiphenyl-4-yl)-ethyl]-pyrrolidine with the (R)-(+)- isomer. The hydrochloride salt was isolated as a white powder. Mass spectrum and 1H-nmr were identical to those of the racemate described in Example 6 above.

### EXAMPLE 8

### General Procedure A

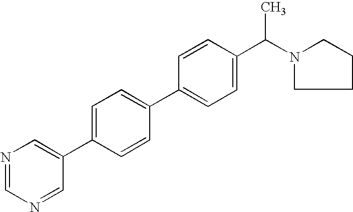

### (S)-(−)-5-[4′-(1-Pyrrolidin-1-ylethyl)-biphenyl-4-yl]-pyrimidine

This enantiomer was prepared according to the procedure of Example 6, replacing racemic (±)-1-[1-(4′-bromobiphenyl-4-yl)-ethyl]-pyrrolidine with the (S)-(+)- isomer. The hydrochloride salt was isolated as a white powder. Mass spectrum and ¹H-nmr were identical to those of the racemate described in Example 6 above.

Using the general procedure A, as described for Example 1 (with the exception of their conversion to a trifluoracetate salt), the following compounds were also prepared:

### EXAMPLE 9

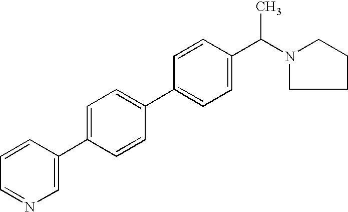

### 3-[4′-(1-Pyrrolidin-1-ylethyl)-biphenyl-4-yl]-pyridine

Mass spectrum (m/z) calcd for C₂₃H₂₄N, 328.19; obsd: 328 (M+).

### EXAMPLE 10

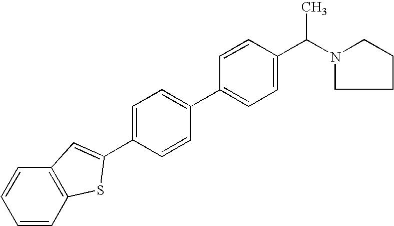

### 1-[1-(4′-Benzo[b]thiophen-2-ylbiphenyl-4-yl)-ethyl]-pyrrolidine

Mass spectrum (m/z) calcd for C₂₆H₂₅NS: 383.17; obsd: 383 (M+).

### EXAMPLE 11

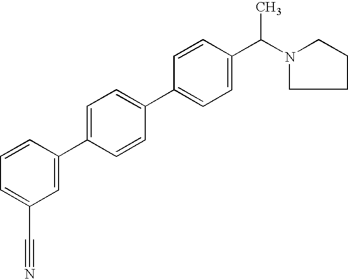

### 4-(1-Pyrrolidin-1-ylethyl)-[1,1′:4′,1″]terphenyl-3″-carbonitrile

Mass spectrum (m/z) calcd for C₂₅H₂₄N₂: 352.19; obsd: 352 (M+).

### EXAMPLE 12

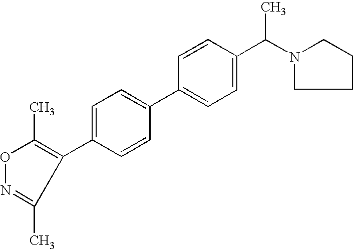

### 3,5-Dimethyl-4-[4′-(1-pyrrolidin-1-ylethyl)-biphenyl-4-yl]-isoxazole

Mass spectrum (m/z) calcd for C₂₃H₂₆N₂O: 346.20; obsd: 346 (M+).

### EXAMPLE 13

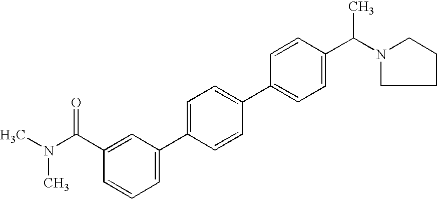

### 4″-(1-Pyrrolidin-1-ylethyl)-[1,1′;4′,1″]terphenyl-3-carboxylic acid dimethylamide

Mass spectrum (m/z) calcd for C₂₇H₃₀N₂O: 398.24; obsd: 399 (M+1).

### EXAMPLE 14

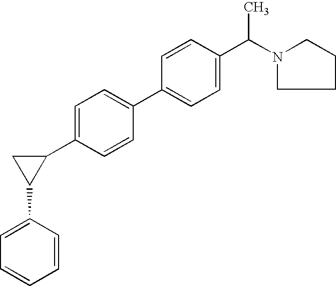

### 1-{1-[4′-(2-Phenylcyclopropyl)-biphenyl-4-yl]-ethyl}-pyrrolidine

Mass spectrum (m/z) calcd for C₂₇H₂₉N, 367.23; obsd: 368 (M+1).

### EXAMPLE 15

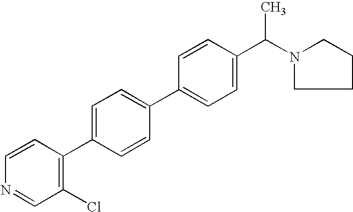

### 3-Chloro-4-[4′-(1-pyrrolidin-1-ylethyl)-biphenyl-4-yl]-pyridine

Mass spectrum (m/z) calcd for C₂₃H₂₃ClN₂: 362.15; obsd: 363 (M+1).

### EXAMPLE 16

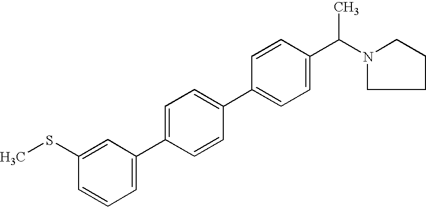

### 1-[1-(3″-Methylsulfanyl-[1,1′;4′,1″]terphenyl-4-yl)-ethyl]-pyrrolidine

Mass spectrum (m/z) calcd for C₂₅H₂₇NS: 373.19; obsd: 374 (M+1).

### EXAMPLE 17

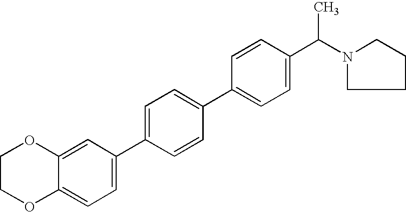

### 1-[1-[4′-(2,3-Dihydro-benzo[1,4]dioxin-6-yl)-biphenyl-4-yl]-ethyl]-pyrrolidine

Mass spectrum (m/z) calcd for C₂₆H₂₇NO₂: 385.20; obsd: 386 (M+1).

### EXAMPLE 18

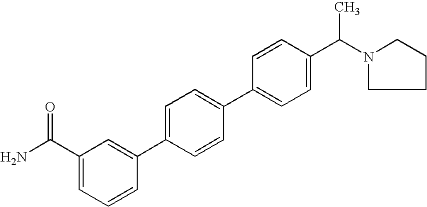

### 4″-(1-Pyrrolidin-1-ylethyl)-[1,1′;4′,1″]terphenyl-3-carboxylic acid amide

Mass spectrum (m/z) calcd for C₂₅H₂₆N₂O: 370.20; obsd: 371 (M+1).

### EXAMPLE 19

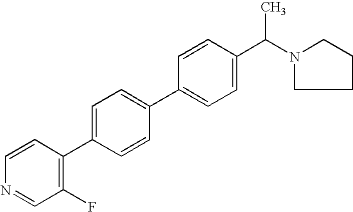

### 3-Fluoro-4-[4′-(1-pyrrolidin-1-ylethyl)-biphenyl-4-yl]-pyridine

Mass spectrum (m/z) calcd for C₂₃H₂₃FN₂: 346.18; obsd: 347 (M+1).

### EXAMPLE 20

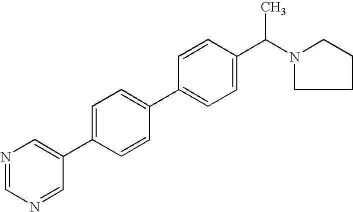

### 5-[4′-(1-Pyrrolidin-1-ylethyl)-biphenyl-4-yl]-pyrimidine

Mass spectrum (m/z) calcd for C₂₂H₂₃N₃: 329.19; obsd: 330 (M+1).

### EXAMPLE 21

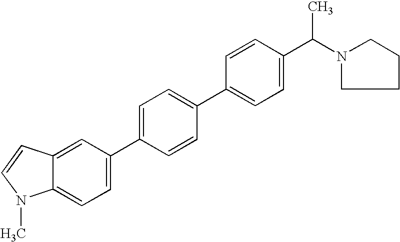

### 1-Methyl-5-[4′-(1-pyrrolidin-1-ylethyl)-biphenyl-4-yl]-1H-indole

Mass spectrum (m/z) calcd for C₂₇H₂₈N₂: 380.23; obsd: 381 (M+1).

### EXAMPLE 22

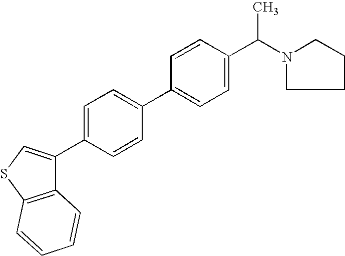

### 1-[1-(4′-Benzo[b]thiophen-3-ylbiphenyl-4-yl)-ethyl]-pyrrolidine

Mass spectrum (m/z) calcd for C₂₆H₂₅NS: 383.17; obsd: 384 (M+1).

### EXAMPLE 23

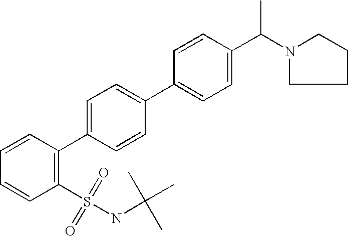

### 4″-(1-Pyrrolidin-1-ylethyl)-[1,1′;4′,1″]terphenyl-2-sulfonic acid tert-butylamide

Mass spectrum (m/z) calcd for C₂₈H₃₄N₂O₂S: 462.23; obsd: 463 (M+1).

### EXAMPLE 24

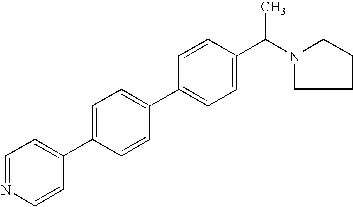

### 4-[4′-(1-Pyrrolidin-1-ylethyl)-biphenyl-4-yl]-pyridine

Mass spectrum (m/z) calcd for C₂₃H₂₄N₂: 328.19; obsd: 329 (M+1).

### EXAMPLE 25

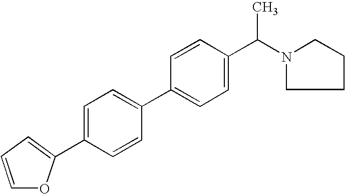

### 1-[1-(4′-Furan-2-ylbiphenyl-4-yl)-ethyl]-pyrrolidine

Mass spectrum (m/z) calcd for C₂₂H₂₃NO: 317.18; obsd: 318 (M+1).

### EXAMPLE 26

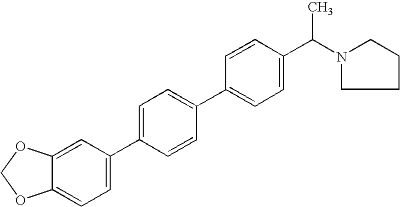

### 1-[1-(4′-Benzo[1,3]dioxol-5-ylbiphenyl-4-yl)-ethyl]-pyrrolidine

Mass spectrum (m/z) calcd for C₂₅H₂₅NO₂: 371.19; obsd: 372 (M+1).

### EXAMPLE 27

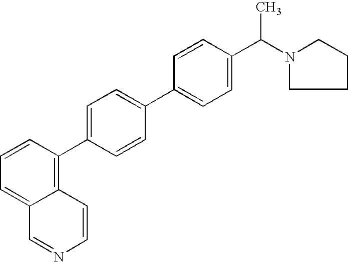

### 5-[4′-(1-Pyrrolidin-1-ylethyl)-biphenyl-4-yl]-isoquinoline

Mass spectrum (m/z) calcd for C₂₇H₂₆N₂: 378.21; obsd: 379 (M+1).

### EXAMPLE 28

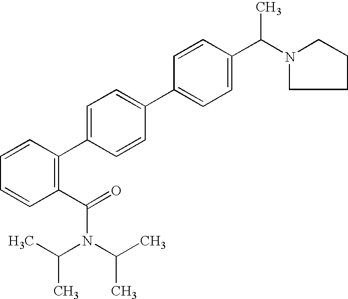

### 4″-(1-Pyrrolidin-1-ylethyl)-[1,1′;4′,1″]terphenyl-2-carboxylic acid diisopropylamide

Mass spectrum (m/z) calcd for C₃₁H₃₈N₂O: 454.30; obsd: 455 (M+1).

### EXAMPLE 29

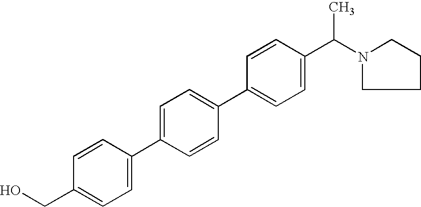

### [4-(1-Pyrrolidin-1-ylethyl)-[1,1′;4′,1″]terphenyl-4″-yl]-methanol

Mass spectrum (m/z) calcd for C₂₅H₂₇NO: 357.21; obsd: 358 (M+1).

### EXAMPLE 30

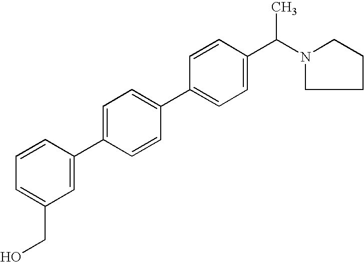

### [4-(1-Pyrrolidin-1-ylethyl)-[1,1′:4′,1″]terphenyl-3″-yl]-methanol

Mass spectrum (m/z) calcd for C₂₅H₂₇NO: 357.21; obsd: 358 (M+1).

### EXAMPLE 31

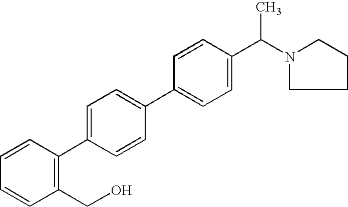

### [4-(1-Pyrrolidin-1-ylethyl)-[1,1′;4′,1″]terphenyl-2-yl]-methanol

Mass spectrum (m/z) calcd for C₂₅H₂₇NO: 357.21; obsd: 358 (M+1).

### EXAMPLE 32

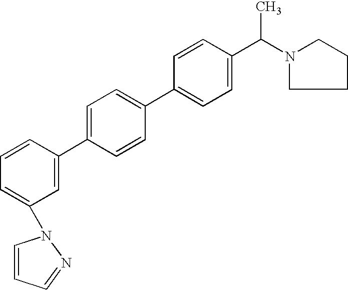

### 1-[4″-(1-Pyrrolidin-1-ylethyl)-[1,1′;4′,1″]terphenyl-3-yl]-1H-pyrazole

Mass spectrum (m/z) calcd for C₂₇H₂₇N₃: 393.22; obsd: 394 (M+1).

### EXAMPLE 33

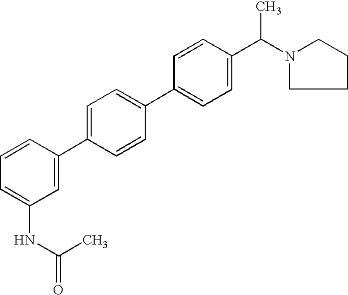

### N-[4″-(1-Pyrrolidin-1-ylethyl)-[1,1′;4′,1″]terphenyl-3-yl]-acetamide

Mass spectrum (m/z) calcd for C₂₆H₂₈N₂O: 384.22; obsd: 385 (M+1).

### EXAMPLE 34

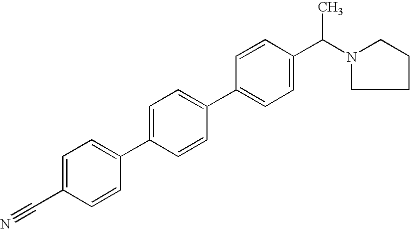

### 4-(1-Pyrrolidin-1-ylethyl)-[1,1′;4′,1″]terphenyl-4″-carbonitrile

Mass spectrum (m/z) calcd for C₂₅H₂₄N₂: 352.19; obsd: 353 (M+1).

### EXAMPLE 35

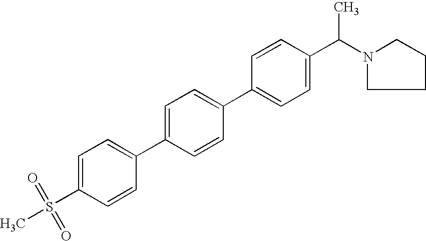

### 1-[1-(4-Methanesulfonyl-[1,1′;4′,1″]terphenyl-4″-yl)-ethyl]-pyrrolidine

Mass spectrum (m/z) calcd for C₂₅H₂₇NO₂S: 405.18; obsd: 406 (M+1).

### EXAMPLE 36

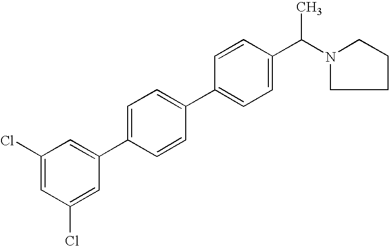

### 1-[1-(3,5-Dichloro-[1,1′;4′,1″]terphenyl-4″-yl)-ethyl]-pyrrolidine

Mass spectrum (m/z) calcd for C₂₄H₂₃Cl₂N, 395.12; obsd: 396 (M+1).

### EXAMPLE 37

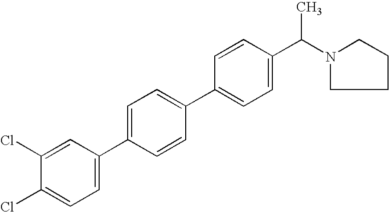

### 1-[1-(3″,4″-Dichloro-[1,1′;4′,1″]terphenyl-4-yl)-ethyl]-pyrrolidine

Mass spectrum (m/z) calcd for C₂₄H₂₃Cl₂N, 395.12; obsd: 396 (M+1).

### EXAMPLE 38

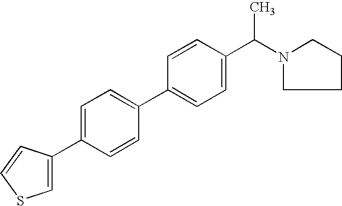

### 1-[1-(4′-Thiophen-3-ylbiphenyl-4-yl)-ethyl]-pyrrolidine

Mass spectrum (m/z) calcd for C₂₂H₂₃NS: 333.16; obsd: 396 (M+1).

### EXAMPLE 39

### General Procedure A

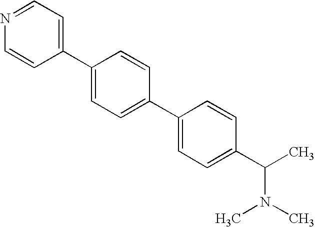

### Dimethyl-[1-(4′-pyridin-4-ylbiphenyl-4-yl)-ethyl]-amine

A mixture of [1-(4′-bromobiphenyl-4-yl)-ethyl]-dimethylamine (152 mg, 0.5 mmol, Intermediate 1) and pyridine-4-boronic acid (74 mg, 0.6 mmol) in 4.0 mL ethanol containing 0.8 mL water was combined with sodium carbonate (212 mg, 2.0 mmol) and tetrakis-(triphenylphosphine)palladium(0) in a 5 mL microwave tube. The mixture was heated in the microwave apparatus for 300 sec at 150° C. After cooling, the mixture was filtered and concentrated in vacuo. The residue was flash chromatographed on silica gel using 1% ammonium hydroxide in a mixture of 5% methanol:95% methylene chloride and the combined product fractions were concentrated in vacuo to a white solid, 62 mg.

Mass spectrum (m/z) calcd for C₂₁H₂₂N₂: 302.42; obsd: 304, 303 (M+1, 100%), 263, 258.

¹H-nmr (CDCl₃, 400 MHz)-δ 1.39 (d, 3H), 2.24 (s, 6H), 3.29 (q, 1H), 7.24 (s, 1H), 7.38 (m, 2H), 7.53-7.59 (m, 3H), 7.70 (s, 4H), 8.65 (dd, 2H).

Using the general procedure A, as described for Example 39 (with the exception of their conversion to a trifluoracetate salt), the following compounds were also prepared:

### EXAMPLE 40

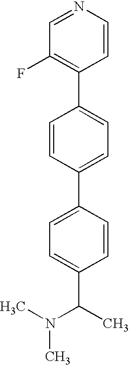

### {1-[4′-(3-Fluoropyridin-4-yl)-biphenyl-4-yl]-ethyl}-dimethylamine

Mass spectrum (m/z) calcd for C₂₁H₂₁FN₂: 320.17; obsd: 321 (M+1).

### EXAMPLE 41

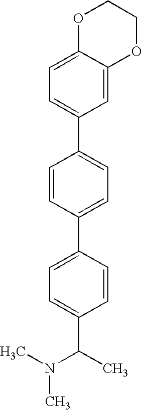

### {1-[4′-(2,3-Dihydro-benzo[1,4]dioxin-6-yl)-biphenyl-4-yl]-ethyl}-dimethylamine

Mass spectrum (m/z) calcd for C₂₄H₂₅NO₂: 359.20; obsd: 360 (M+1).

### EXAMPLE 42

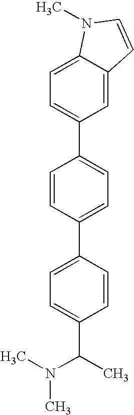

### Dimethyl-{1-[4′-(1-methyl-1H-indol-5-yl)-biphenyl-4-yl]-ethyl}-amine

Mass spectrum (m/z) calcd for C₂₅H₂₆N₂: 354.49. obsd: 355 (M+1).

### EXAMPLE 43

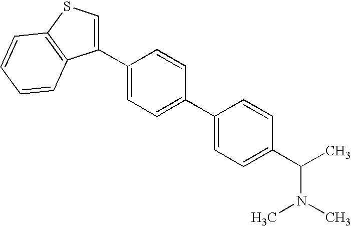

### [1-(4′-Benzo[b]thiophen-3-ylbiphenyl-4-yl)-ethyl]-dimethylamine

Mass spectrum (m/z) calcd for C₂₄H₂₃NS: 357.52. obsd: 358 (M+1).

### EXAMPLE 44

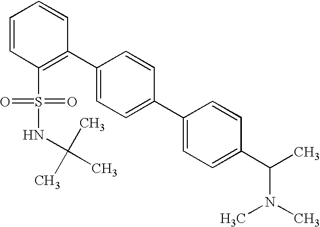

### 4″-(1-Dimethylaminoethyl)-[1,1′;4′,1″]terphenyl-2-sulfonic acid tert-butylamide

Mass spectrum (m/z) calcd for C₂₆H₃₂N₂O₂S: 436.62. obsd: 437 (M+1).

### EXAMPLE 45

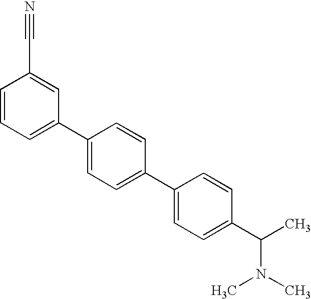

### 4″-(1-Dimethylaminoethyl)-[1,1′;4′,1″]terphenyl-3-carbonitrile

Mass spectrum (m/z) calcd for C₂₃H₂₂N₂: 326.44. obsd: 327 (M+1).

### EXAMPLE 46

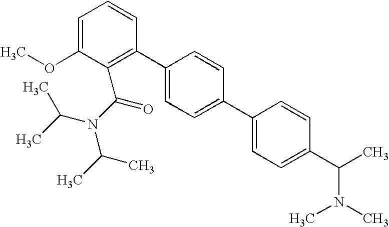

### 4″-(1-Dimethylaminoethyl)-3-methoxy-[1,1′;4′,1″]terphenyl-2-carboxylic acid diisopropylamide

Mass spectrum (m/z) calcd for C₃₀H₃₈N₂O₂: 458.64. obsd: 460 (M+1).

### EXAMPLE 47

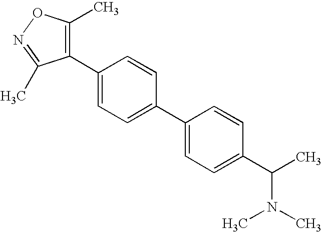

### {1-[4′-(3,5-Dimethylisoxazol-4-yl)-biphenyl-4-yl]-ethyl}-dimethylamine

Mass spectrum (m/z) calcd for C₂₁H₂₄N₂O: 320.43; obsd: 321 (M+1).

### EXAMPLE 48

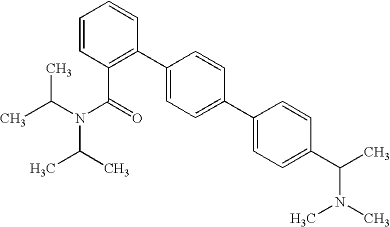

### 4″-(1-Dimethylaminoethyl)-[1,1′;4′,1″]terphenyl-2-carboxylic acid diisopropylamide

Mass spectrum (m/z) calcd for C₂₉H₃₆N₂O: 428.28; obsd: 429 (M+1).

### EXAMPLE 49

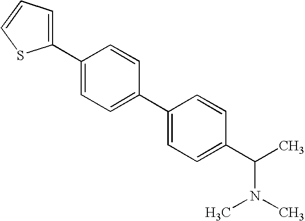

### Dimethyl-[1-(4′-thiophen-2-ylbiphenyl-4-yl)-ethyl]-amine

Mass spectrum (m/z) calcd for C₂₀H₂₁NS: 307.14; obsd: 308 (M+1).

### EXAMPLE 50

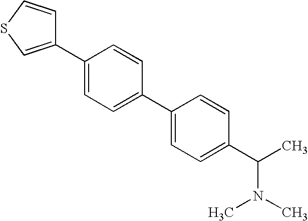

### Dimethyl-[1-(4′-thiophen-3-ylbiphenyl-4-yl)-ethyl]-amine

Mass spectrum (m/z) calcd for C₂₀H₂₁NS: 307.14; obsd: 308 (M+1).

### EXAMPLE 51

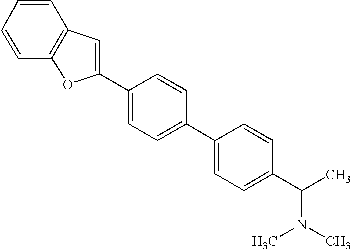

### [1-(4′-Benzofuran-2-ylbiphenyl-4-yl)-ethyl]-dimethylamine

Mass spectrum (m/z) calcd for C₂₄H₂₃NO: 341.18; obsd: 342 (M+).

### EXAMPLE 52

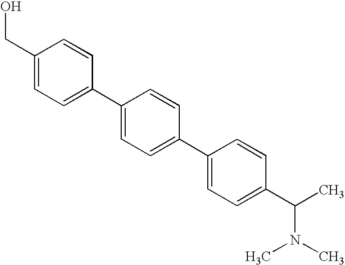

### [4-(1-Dimethylaminoethyl)-[1,1′;4′,1″]terphenyl-4″-yl]-methanol

Mass spectrum (m/z) calcd for C₂₃H₂₅NO: 331.19; obsd: 332 (M+1).

### EXAMPLE 53

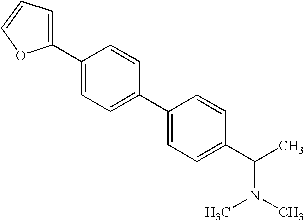

### [1-(4′-Furan-2-ylbiphenyl-4-yl)-ethyl]-dimethylamine

Mass spectrum (m/z) calcd for C₂₀H₂₁NO: 291.16; obsd: 292 (M+1).

### EXAMPLE 54

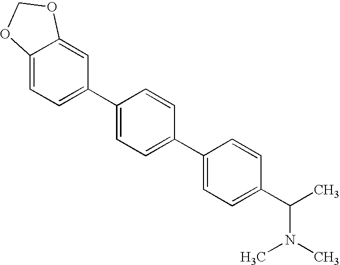

### [1-(4′-Benzo[1.3]dioxol-5-ylbiphenyl-4-yl)-ethyl]-dimethylamine

Mass spectrum (m/z) calcd for C₂₃H₂₃NO₂: 345.17; obsd: 346 (M+1).

### EXAMPLE 55

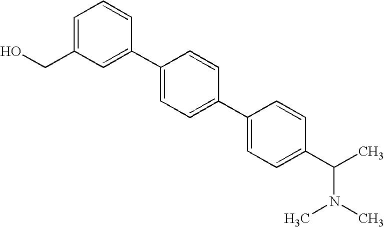

### [4″-(1-Dimethylaminoethyl)-[1,1′;4′,1″]terphenyl-3-yl]-methanol

Mass spectrum (m/z) calcd for C₂₃H₂₅NO: 331.19; obsd: 332 (M+1).

### EXAMPLE 56

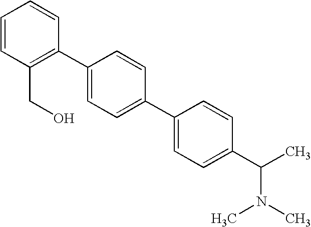

### [4″-(1-Dimethylaminoethyl)-[1,1′;4′,1″]terphenyl-2-yl]-methanol

Mass spectrum (m/z) calcd for C₂₃H₂₅NO: 331.19; obsd: 332 (M+1).

### EXAMPLE 57

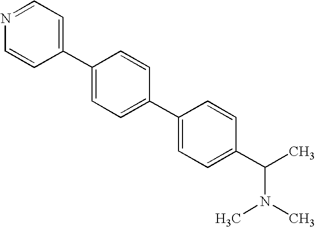

### Dimethyl-[1-(4′-pyridin-4-ylbiphenyl-4-yl)-ethyl]-amine

Mass spectrum (m/z) calcd for C₂₁H₂₂N₂: 302.18; obsd: 303 (M+1).

### EXAMPLE 58

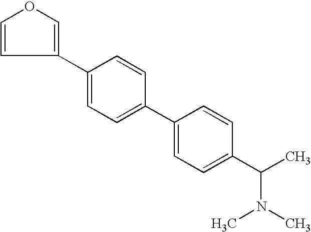

### [1-(4′-Furan-3-ylbiphenyl-4-yl)-ethyl]-dimethylamine

Mass spectrum (m/z) calcd for C₂₀H₂₁NO: 291.16; obsd: 292 (M+1).

### EXAMPLE 59

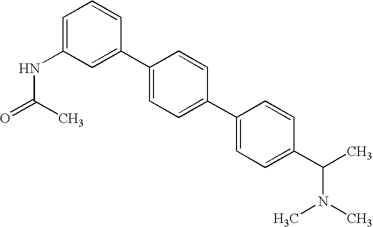

### N-[4″-(1-Dimethylaminoethyl)-[1,1′;4′,1″]terphenyl-3-yl]-acetamide

Mass spectrum (m/z) calcd for C₂₄H₂₆N₂O: 358.20; obsd: 359 (M+1).

### EXAMPLE 60

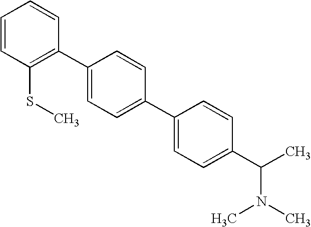

### Dimethyl-[1-(2-methylsulfanyl-[1,1′;4′,1″]terphenyl-4″-yl)-ethyl]-amine

Mass spectrum (m/z) calcd for C₂₃H₂₅NS: 347.17; obsd: 348 (M+).

### EXAMPLE 61

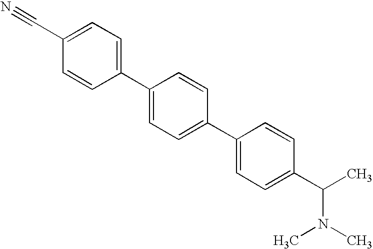

### 4-(1-Dimethylaminoethyl)-[1,1′;4′,1″]terphenyl-4″-carbonitrile

Mass spectrum (m/z) calcd for C₂₃H₂₂N₂: 326.18; obsd: 327 (M+1).

### EXAMPLE 62

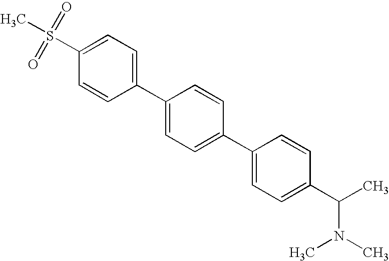

### [1-(4-Methanesulfonyl-[1,1′;4′,1″]terphenyl-4″-yl)-ethyl]-dimethylamine

Mass spectrum (m/z) calcd for C₂₃H₂₅NO₂S: 379.16; obsd: 380 (M+1).

### EXAMPLE 63

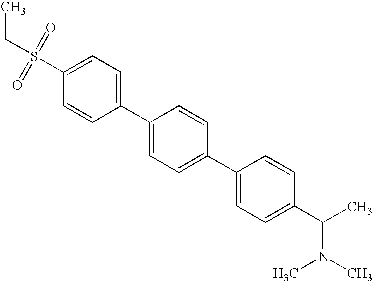

### [1-(4-Ethanesulfonyl-[1,1′;4′,1″]terphenyl-4″-yl)-ethyl]-dimethylamine

Mass spectrum (m/z) calcd for C₂₄H₂₇NO₂S: 393.18; obsd: 394 (M+1).

### EXAMPLE 64

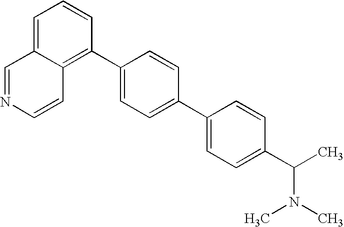

### [1-(4′-Isoquinolin-5-ylbiphenyl-4-yl)-ethyl]-dimethylamine

Mass spectrum (m/z) calcd for C₂₅H₂₄N₂:352.19; obsd: 353 (M+1).

### EXAMPLE 65

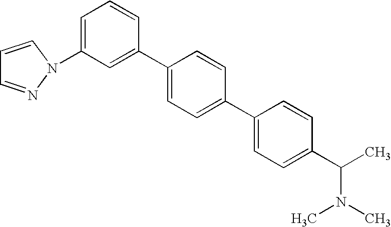

### Dimethyl-[1-(3-pyrazol-1-yl-[1,1′;4′,1″]terphenyl-4″-yl)-ethyl]-amine

Mass spectrum (m/z) calcd for C₂₅H₂₅N₃: 367.2; obsd: 368 (M+1).

### EXAMPLE 66

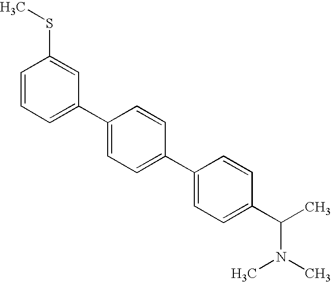

### Dimethyl-[1-(3-methylsulfanyl-[1,1′;4′,1″]terphenyl-4″-yl)-ethyl]-amine

Mass spectrum (m/z) calcd for C₂₃H₂₅NS: 347.17; obsd: 348 (M+1).

### EXAMPLE 67

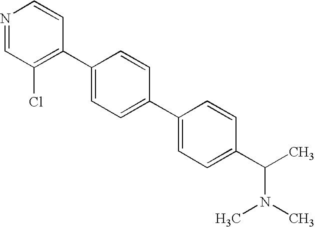

### {1-[4′-(3-Chloropyridin-4-yl)-biphenyl-4-yl]-ethyl}-dimethylamine

Mass spectrum (m/z) calcd for C₂₁H₂₁ClN₂: 336.14; Obsd: 337 (M+1).

### EXAMPLE 68

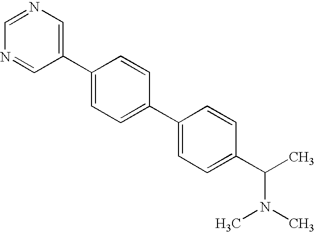

### Dimethyl-[1-(4′-Pyrimidin-5-ylbiphenyl-4-yl)-ethyl]-amine

Mass spectrum (m/z) calcd for C₂₀H₂₁N₃: 303.14; obsd: 304 (M+1).

### EXAMPLE 69

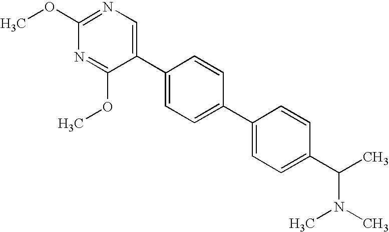

### {1-[4′-(2,4-Dimethoxypyrimidin-5-yl)-biphenyl-4-yl]-ethyl}-dimethylamine

Mass spectrum (m/z) calcd for C₂₂H₂₅N₃O₂: 363.19; obsd: 364 (M+1).

### EXAMPLE 70

### General Procedure B

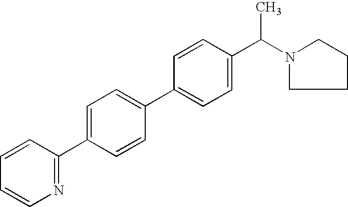

### (R)-(±)-2-[4′-(1-Pyrrolidin-1-ylethyl)-biphenyl-4-yl]-pyridine

A mixture of (R)-(±)-1-{1-[4′-(4,4,5,5-tetramethyl-[1,3,2]dioxaborolan-2-yl)-biphenyl-4-yl]-ethyl}-pyrrolidine (200 mg, 0.5 mmol, intermediate 3), 2-bromopyridine (95 mg, 0.6 mmol), sodium carbonate (212 mg, 4.0 mmol) and tetrakis(triphenylphosphine)palladium(0) in 4.0 mL of water containing 0.8 mL ethanol was stirrer and degassed in a 5.0 mL microwave tube and heated at 150° for 300 sec. The mixture was cooled to room temperature and filtered through d.e., the filtrate was concentrated in vacuo to a light red solid. The crude material was flash chromatographed on silica gel using a gradient of 0-5% MeOH in methylene chloride to give, after removal of the solvents, 53 mg of white solid. Conversion to the HCl salt gave a white powder.

Mass spectrum (m/z) calcd for C₂₃H₂₄N₂: 328; obsd: 330, 329 (M+1).

¹H-nmr (CDCl₃, 400 MHz)-δ 1.48 (d, 3H), 1.81 (bs, 4H), 2.50 (bs, 2H), 2.67 (bs, 2H), 3.33 (m, 1H), 7.24 (m, 1H), 7.45 (m, 2H), 7.59 (m, 2H), 7.70 (m, 2H), 7.76 (m, 2H), 8.05 (d, 2H), 8.69 (m, 1H).

### EXAMPLE 71

### General Procedure B

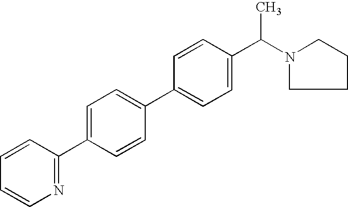

### (±)-2-[4′-(1-Pyrrolidin-1-ylethyl)-biphenyl-4-yl]-pyridine

This was prepared as in Example 70, replacing (R)-(±)-1-{1-[4′-(4,4,5,5-tetramethyl-[1,3,2]dioxaborolan-2-yl)-biphenyl-4-yl]-ethyl}-pyrrolidine with the racemic boronate (intermediate 3), to produce the hydrochloride salt as a white powder.

Mass spectrum (m/z) calcd for C₂₃H₂₄N₂: 328; obsd: 330, 329 (M+1).

### EXAMPLE 72

### General Procedure C

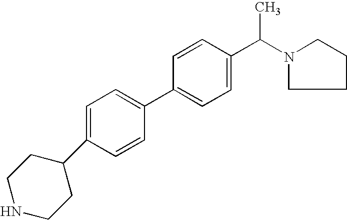

### (R)-(±)-4-[4′-(1-Pyrrolidin-1-ylethyl)-biphenyl-4-yl]-piperidine

A mixture of (R)-(±)-4-[4′-(1-pyrrolidin-1-ylethyl)-biphenyl-4-yl]-pyridine (61 mg, mmol) and platinum (II) oxide (20 mg) in 5 mL of methanol was hydrogenated on a Parr shaker apparatus at an initial hydrogen pressure of 45 psi for 4 hours. The reaction was filtered through d.e., the solids were washed with additional methanol and the solvent removed in vacuo to give a colorless gummy residue, 49 mg. This was converted as above to the hydrochloride salt.

Mass spectrum (m/z) calcd for C₂₃H₃₀N₂: 334.50; obsd: 335 (M+1).

¹H-nmr (CDCl₃, 400 MHz)-δ 1.43 (d, 3H), 1.65-1.88 (m, 10H), 2.40 (m, 2H), 2.57 (m, 2H), 2.65 (m, 1H), 2.76 (t, 1H), 3.22 (m, 2H), 7.26 (m, 3H), 7.38 (m, 2H), 7.54 (m, 3H).

### EXAMPLE 73

### General Procedure C

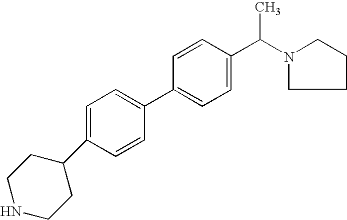

### (S)-(−)4-[4′-(1-Pyrrolidin-1-ylethyl)-biphenyl-4-yl]-piperidine

This was prepared in the same manner as described in Example 72, beginning with (S)-(−)-4-[4′-(1-pyrrolidin-1-ylethyl)-biphenyl-4-yl]-pyridine to produce the hydrochloride salt as a white powder.

Mass spectrum (m/z) calcd for C₂₃H₃₀N₂: 334.50; obsd: 335 (M+1).

### EXAMPLE 74

### General Procedure C

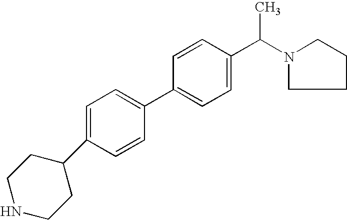

### (±)-4-[4′-(1-Pyrrolidin-1-yl-ethyl)-biphenyl-4-yl]-piperidine

This was prepared in the same manner as described in Example 72, beginning with racemic 4-[4′-(1-pyrrolidin-1-ylethyl)-biphenyl-4-yl]-pyridine to produce the hydrochloride salt as a white powder.

Mass spectrum (m/z) calcd for C₂₃H₃₀N₂: 334.50; obsd: 335 (M+1).

### EXAMPLE 75

### General Procedure C

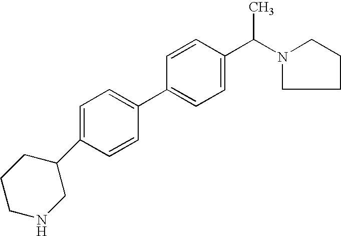

### (R)-3-[4′-(1-Pyrrolidin-1-ylethyl)-biphenyl-4-yl]-piperidine

Prepared as in Example 72 above from 163 mg of (R)-(±)-3-[4′-(1-pyrrolidin-1-ylethyl)-biphenyl-4-yl]-pyridine to give a colorless gummy residue, 141 mg. Converted in the manner previously described to the hydrochloride salt, isolated as a white powder.

Mass spectrum (m/z) calcd for C₂₃H₃₀N₂: 334.50; obsd: 335 (M+1).

¹H-nmr (CDCl₃, 400 MHz)-δ 1.42 (d, 3H), 1.63 (t, 2H), 1.76 (m, 4H), 2.01 (m, 1H), 2.11 (bs, 1H), 2.40 (m, 2H), 2.58 (m, 2H), 2.69 (m, 2H), 3.12 (d, 1H), 3.23 (m, 2H), 7.23 (m, 3H), 7.37 (m, 2H), 7.48 (m, 3H).

### EXAMPLE 76

### General Procedure C

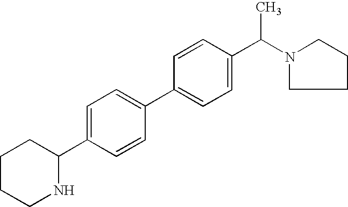

### (R)-2-[4′-(1-Pyrrolidin-1-ylethyl)-biphenyl-4-yl]-piperidine

Prepared as in Example 72 above from 53 mg of (R)-2-[4′-(1-pyrrolidin-1-ylethyl)-biphenyl-4-yl]-pyridine to give a colorless gummy residue, 45 mg. This was converted to the hydrochloride salt as a white powder.

Mass spectrum (m/z) calcd for C₂₃H₃₀N₂: 334.50; obsd: 335 (M+1).

¹H-nmr (CDCl₃, 400 MHz)-δ 1.43 (d, 3H), 1.54 (m, 3H), 1.65-1.90 (m, 6H), 1.92 (m, 1H), 2.41 (m, 2H), 2.56 (m, 2H), 2.81 (t, 1H), 3.22 (t, 2H), 3.62 (m, 1H), 7.11-7.26 (m, 1H), 7.40 (m, 4H), 7.53 (t, 4H).

### EXAMPLE 77

### General Procedure D

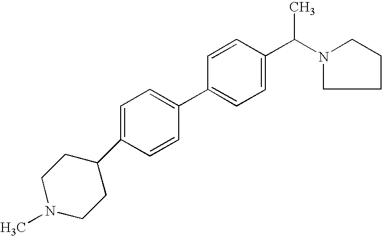

### (R)-1-Methyl-4-[4′-(1-Pyrrolidin-1-ylethyl)-biphenyl-4-yl]-piperidine

A mixture of (R)-(±)-4-[4′-(1-pyrrolidin-1-ylethyl)-biphenyl-4-yl]-piperidine (30 mg, 0.09 mmol, Example 72) and 37% aqueous formaldehyde (0.12 mL, 1.5 mmol) in 2 mL methanol was stirred at room temperature for two hours, then treated with sodium triacetoxyborohydride (95 mg, 0.45 mmol) and stirred overnight. The solvent was removed in vacuo and the residue partitioned with saturated aqueous Na₂CO₃ and methylene chloride. The organic extracts were combined, washed with water and dried over MgSO₄, then concentrated in vacuo to a white solid, 24 mg. This material was redissolved in ethyl acetate and treated with 1.0 M HCl in diethyl ether to give the hydrochloride salt as a white solid, 23 mg.

Mass spectrum (m/z) calcd for C₂₄H₃₂N₂: 348.53; obsd: 350, 349 (M+1).

¹H-nmr (CDCl₃, 400 MHz)-δ 1.40 (d, 3H), 1.75 (m, 4H), 1.83 (m, 4H), 2.04 (dt, 2H), 2.31 (s, 3H), 2.37 (m, 2H), 2.54 (m, 3H), 2.97 (d, 2H), 3.19 (q, 1H), 7.25 (m, 2H), 7.35 (m, 2H), 7.49 (m, 4H).

### EXAMPLE 78

### General Procedure D

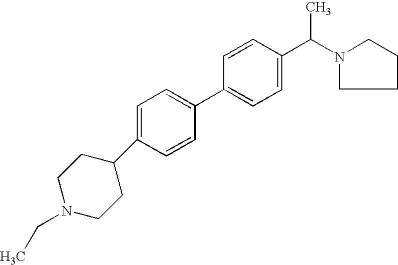

### (R)-1-Ethyl-4-[4′-(1-pyrrolidin-1-ylethyl)-biphenyl-4-yl]-piperidine

This compound was prepared in the same manner as described in Example 77, replacing the formaldehyde solution with acetaldehyde. Thus, 30 mg of (R)-(±)-4-[4′-(1-pyrrolidin-1-ylethyl)-biphenyl-4-yl]-piperidine gave the hydrochloride salt as a white solid, 26 mg.

Mass spectrum (m/z) calcd for C₂₅H₃₄N₂: 362.56; obsd: 364, 363 (M+1).

1H-nmr (CDCl₃, 400 MHz)-δ 1.11 (t, 3H), 1.41 (d, 3H), 1.72-1.86 (m, 9H), 2.01 (dt, 2H), 2.31-2.64 (m, 6H), 3.08 (d, 2H), 3.20 (m, 1H), 7.26 (m, 2H), 7.35 (m, 2H), 7.49 (m, 4H).

### EXAMPLE 79

### General Procedure E

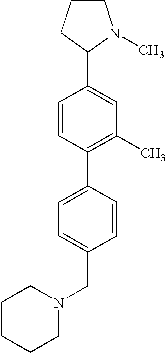

### 1-[2′-Methyl-4′-(1-methylpyrrolidin-2-yl)-biphenyl-4-ylmethyl]-piperidine

To a slurry of 2′-methyl-4′-(1-methylpyrrolidin-2-yl)-biphenyl-4-carboxaldehyde (100 mg, 0.36 mmol, intermediate 5) in 15 mL ethanol, stirred at room temperature, was added piperidine (61 mg, 0.72 mmol), followed by dropwise addition of titanium (IV) isopropoxide (205 mg, 0.72 mmol). The resulting yellow solution was stirred for 22 hr and treated with sodium borohydride (21 mg, 0.72 mmol)—some foaming was noted. The solution was stirred another 5 hr, then quenched with water and ethyl acetate. The organic layer was combined with additional ethyl acetate extractions of the aqueous layer, washed with saturated aqueous NaCl and dried with MgSO₄. The solvent was removed in vacuo to give a light yellow foam, 81 mg. This was flash chromatographed on silica gel using chloroform. The product fractions were combined and concentrated to a clear tan oil, 48 mg. The oil in a minimal amount of ethyl acetate was treated with 0.5 mL of 1.0 M HCl in diethyl ether, stirred for 2 hr and filtered to give a white solid which was dried under vacuum, 28 mg. M.p. 237.1-240° C.

Mass spectrum (m/z) calcd for C₂₄H₃₂N₂: 348.53; obsd: 349 (M+1).

¹H-nmr (CDCl₃, 400 MHz, free base): δ 1.43 (bm, 2H), 1.58 (m, 4H), 1.79 (m, 2H), 1.90 (m, 1H), 2.20 (s, 3H), 2.25 (s, 3H), 2.26 (m, 2H), 2.40 (bs, 4H), 3.01 (t, 1H), 3.24 (t, 1H), 3.49 (s, 2H), 7.16 (s, 1H), 7.22 (d, 2H), 7.24 (d, 2H), 7.32 (d, 2H).

### EXAMPLE 80

### General Procedure F

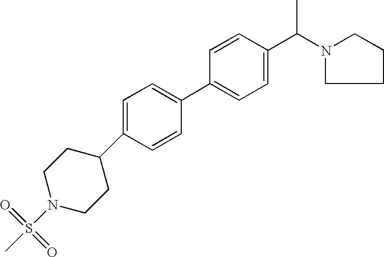

### 1-Methanesulfonyl-4-[4′-(1-pyrrolidin-1-ylethyl)-biphenyl-4-yl]-piperidine

A mixture of (±)-4-[4′-(1-pyrrolidin-1-yl-ethyl)-biphenyl-4-yl]-piperidine (168 mg. 0.5 mmol) and triethylamine (0.14 mL, 1.0 mmol) in 10 mL CH₂Cl₂ was treated with methanesulfonyl chloride (0.047 mL, 0.6 mmol) and stirred at rt overnight. The reaction mixture was washed with water, aqueous NaCl and dried over Na₂SO₄. Removal of the solvent in vacuo gave a white solid which was triturated with EtOAc and filtered. After drying at rt, the product was obtained as a white solid, 142 mg. The free base was converted as described previously to the hydrochloride salt.

Mass spectrum (m/z) calcd for C₂₄H₃₂N₂O₂S: 412.59; obsd: 413 (M+1).

1H-nmr (CDCl₃, 400 MHz, free base): δ 1.80-2.04 (m, 10H), 2.15 (q, 1H), 2.31 (q, 1H), 2.60-2.80 (m, 3H), 2.81 (s, 3H), 2.89 (m, 1H), 3.30 (m, 1H), 3.92-4.03 (m, 4H), 7.23-7.28 (m, 2H), 7.49-7.56 (m, 2H), 7.58-7.66 (m, 2H), 7.71 (d, 2H).

### EXAMPLE 81

### General Procedure G

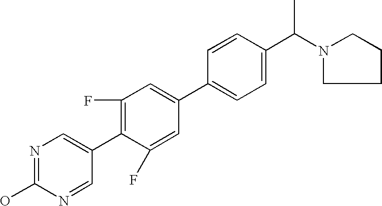

### 5-[3,5-Difluoro-4′-(1-pyrrolidin-1-ylethyl)-biphenyl-4-yl]-pyrimidin-2-ol

A mixture of 5-[3,5-difluoro-4′-(1-pyrrolidin-1-ylethyl)-biphenyl-4-yl]-2-methoxy-pyrimidine (25 mg, the title compound of Example 85) in acetic acid (1.5 mL) was treated with 48% hydrobromic acid and stirred at rt for 72 h, at which time the reaction was judged to be complete. The soldvent was removed in vacuo, dissolved with water and made basic with dilute aqueous NaOH, exrtracted with methylene chloride. The organic extract was washed with water and dried with Na₂SO₄. Removal of the solvent gave a white solid, 21 mg. This was converted to the hydrochloride salt in the usual manner.

Mass spectrum (m/z) calcd for C₂₂H₂₁F₂N₃O: 381.42; obsd: 382 (M+1).

1H-nmr (CH₃OD, 400 MHz, free base): δ 1.43 (d, 3H), 1.79 (m, 4H), 2.40 (m, 2H), 2.62 (m, 2H), 3.29 (m, 1H+ CH₃OH), 7.34 (d, 2H), 7.45 (m, 2H), 7.63 (d, 2H), 8.29 (s, 2H).

### EXAMPLE 82

### General Procedure G

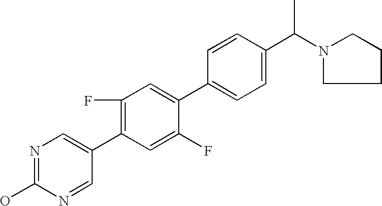

### 5-[2,5-Difluoro-4′-(1-pyrrolidin-1-ylethyl)-biphenyl-4-yl]-pyrimidin-2-ol

Prepared as in Example 81, starting with 13 mg of 5-[2,5-difluoro-4′-(1-pyrrolidin-1-ylethyl)-biphenyl-4-yl]-2-methoxy-pyrimidine (Example 87) and 0.35 mL of 48% HBr in 1.0 mL of acetic acid, to give the hydrochloride as a white solid, 10 mg.

Mass spectrum (m/z) calcd for C₂₂H₂₁F₂N₃O: 381.42; obsd: 382 (M+1).

1H-nmr (CH₃OD, 400 MHz, free base): δ 1.44 (d, 3H), 1.80 (m, 4H), 2.41 (m, 2H), 2.63 (m, 2H), 3.28 (m, 1H), 7.29 (m, 2H), 7.43 (m, 2H), 7.53 (m, 2H), 8.43 (s, 2H).

### EXAMPLE 83

### General Procedure G

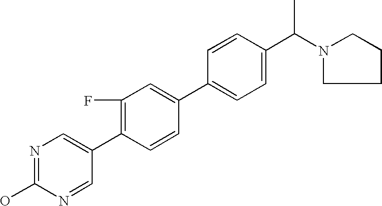

### 5-[3-Fluoro-4′-(1-pyrrolidin-1-ylethyl)-biphenyl-4-yl]-pyrimidin-2-ol

Prepared as in Example 81, starting with 32 mg of 5-[3-fluoro-4′-(1-pyrrolidin-1-ylethyl)-biphenyl-4-yl]-2-methoxypyrimidine (Example 89), and 0.5 mL of 48% HBr in 1.5 mL acetic acid, to give the HCl salt as a pale yellow solid, 27 mg.

Mass spectrum (m/z) calcd for C₂₂H₂₂FN₃O: 363.43; obsd: 364 (M+1).

1H-nmr (CH₃OD, 400 MHz, free base): δ 1.44 (d, 3H), 1.78 (m, 4H), 2.40 (m, 2H), 2.62 (m, 2H), 3.28 (m, 1H), 7.41-7.49 (m, 6H), 7.61 (d, 2), 8.41 (s, 2H).

### EXAMPLE 84

### General Procedure A

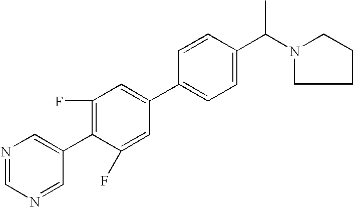

### 5-[3,5-Difluoro-4′-(1-pyrrolidin-1-ylethyl)-biphenyl-4-yl]-pyrimidine

Prepared as in Example 1, starting with 44 mg of 1-[1-(4′-bromo-3′,5′-difluoro-biphenyl-4-yl)-ethyl]-pyrrolidine (Intermediate 8) and 22 mg (0.18 mmol) of pyrimidine-5-boronic acid, to give the hydrochloride salt as a white solid.

Mass spectrum (m/z) calcd for C₂₂H₂₁F₂N₃: 365.43; obsd: 366(M+1).

1H-nmr (CDCl₃, 400 MHz, free base): δ 1.43 (bs (3H), 1.62 (bs, 2H), 1.78 (m, 3H), 2.42 (m, 2H), 2.58 (m, 2H), 3.26 (m, 1H), 7.28 (m, 2H), 7.47 (m, 2H), 7.53 (m, 2H), 8.91 (s, 2H), 9.22 (s, 1H).

### EXAMPLE 85

### General Procedure A

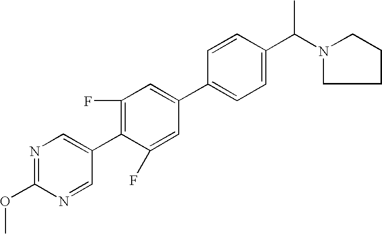

### 5-[3,5-Difluoro-4′-(1-pyrrolidin-1-ylethyl)-biphenyl-4-yl]-2-methoxypyrimidine

Prepared as in Example 1, starting with 130 mg of 1-[1-(4′-bromo-3′,5′-difluoro-biphenyl-4-yl)-ethyl]-pyrrolidine (Intermediate 8) and 115 mg (0.75 mmol) of 2-methoxypyrimidine-5-boronic acid, to give the hydrochloride salt as an off-white solid.

Mass spectrum (m/z) calcd for C₂₃H₂₃F₂N₃O: 395.45; obsd: 396 (M+1).

1H-nmr (CDCl₃, 400 MHz, free base): δ 1.42 (d, 3H), 1.62 (bs, 1H), 1.77 (m, 3H), 2.39 (m, 2H), 2.56 (m, 2H), 3.23 (m, 1H), 4.07 (s, 3H), 7.24 (m, 2H), 7.39-7.51 (m, 4H), 8.68 (s, 2H).

### EXAMPLE 86

### General Procedure A

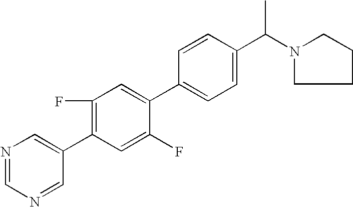

### 5-[2,5-Difluoro-4′-(1-pyrrolidin-1-ylethyl)-biphenyl-4-yl]-pyrimidine

Prepared as in Example 1, starting with 46 mg (0.12 mmol) of 1-[1-(4′-bromo-2′,5′-difluoro-biphenyl-4-yl)-ethyl]-pyrrolidine (intermediate 9) and 22 mg (0.18 mmol) of pyrimidine-5-boronic acid to give, after conversion to the hydrochloride salt, a white solid, 6 mg.

Mass spectrum (m/z) calcd for C₂₂H₂₁F₂N₃: 365.42; obsd: 366 (M+1).

1H-nmr (CDCl₃, 400 MHz, free base): δ 1.42 (d, 3H), 1.71 (bs, 1H), 1.77 (m, 4H), 2.41 (m, 2H), 2.57 (m, 2H), 3.24 (q, 1H), 7.23-7.33 (m, 2H), 7.44 (d, 2H), 7.53 (m, 2H), 8.96 (s, 2H), 9.23 (s, 1H)

### EXAMPLE 87

### General Procedure A

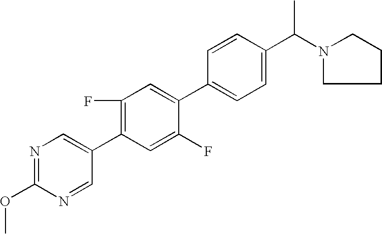

### 5-[2,5-Difluoro-4′-(1-pyrrolidin-1-ylethyl)-biphenyl-4-yl]-2-methoxy-pyrimidine

Prepared according to the method of Example 1 starting with 142 mg (0.39 mmol) of 1-[1-(4′-bromo-2′,5′-difluoro-biphenyl-4-yl)-ethyl]-pyrrolidine (intermediate 9) and 92 mg (0.60 mmol) of 2-methoxypyrimidine-5-boronic acid to give 40 mg of the hydrochloride salt as a white solid.

Mass spectrum (m/z) calcd for C₂₃H₂₃F₂N₃O: 395.45; obsd: 396 (M+1).

1H-nmr (CDCl₃, 400 MHz, free base): δ 1.42 (d, 3H), 1.61 (m, 1H), 1.78 (m, 3H), 2.42 (m, 2H), 2.56 (m, 2H), 3.23 (m, 1H), 4.07 (s, 3H), 7.18-7.31 (m, 2H), 7.43 (m, 2H), 7.50 (m, 2H), 8.73 (s, 2H).

### EXAMPLE 88

### General Procedure A

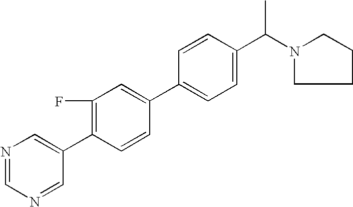

### 5-[3-Fluoro-4′-(1-pyrrolidin-1-ylethyl)-biphenyl-4-yl]-pyrimidine

Prepared according to the method described in Example 1, starting with 452 mg (1.3 mol) of 1-[1-(4′-bromo-3′-fluoro-biphenyl-4-yl)-ethyl]-pyrrolidine (Intermediate 10) and 242 mg (1.95 mmole) of pyrimidine-5-boronic acid to give 247 mg of a tan gummy residue which was converted to the hydrochloride salt in the manner described previously.

Mass spectrum (m/z) calcd for C₂₂H₂₂FN₃: 347.43; obsd: 364 (M+1).

1H-nmr (CDCl₃, 400 MHz, free base): δ 1.44 (d, 3H), 1.61 (m, 1H), 1.79 (m, 3H), 2.42 (m, 2H), 2.58 (m, 2H), 3.24 (m, 1H), 7.38-7.63 (m, 7H), 8.99 (m, 2H), 9.24 (s, 1H).

### EXAMPLE 89

### General Procedure A

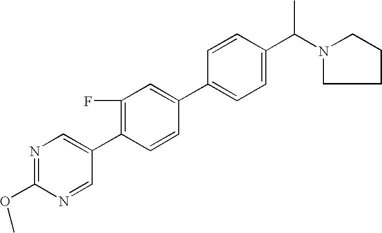

### 5-[3-Fluoro-4′-(1-pyrrolidin-1-ylethyl)-biphenyl-4-yl]-2-methoxypyrimidine

Prepared according to the method described in Example 1, starting with 180 mg (0.52 mol) of 1-[1-(4′-bromo-3′-fluoro-biphenyl-4-yl)-ethyl]-pyrrolidine (Intermediate 10) and 120 mg (0.78 mmole) of 2-methoxypyrimidine-5-boronic acid to give the free base as a pale yellow gum, 102 mg.

Mass spectrum (m/z) calcd for C₂₃H₂₄FN₃O: 377.46; obsd: 378 (M+1).

1H-nmr (CDCl₃, 400 MHz, free base): δ 1.45 (d, 3H), 1.62 (m, 1H), 1.80 (m, 3H), 2.45 (m, 2H), 2.60 (m, 2H), 3.28 (m, 1H), 4.08 (s, 3H), 7.31-7.59 (m, 7H), 8.76 (s, 2H).

### EXAMPLE 90

### General Procedure B

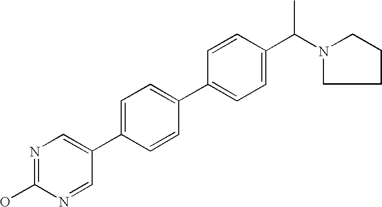

### 5-[4′-(1-Pyrrolidin-1-ylethyl)-biphenyl-4-yl]-pyrimidin-2-ol

This compound was prepared according to the method described in example 70, starting with 100 mg (0.26 mmol) of (±)1-[1-[4′-(4,4,5,5-tetramethyl-[1,3,2]dioxaborolan-2-yl)-biphenyl-4-yl]-ethyl]-pyrrolidine (Intermediate 3) and 68 mg (0.39 mmol) of 2-hydoxy-5-bromopyrimidine. The hydrochloride salt was isolated as a pale yellow solid, 4 mg.

Mass spectrum (m/z) calcd for C₂₂H₂₃N₃O: 345.44; obsd: 346 (M+1).

1H-nmr (CH₃OD, 400 MHz, free base): δ 1.46 (d, 3H), 1.81 (m, 4H), 2.44 (m, 2H), 2.66 (m, 2H), 3.31 (m, 1H), 7.42 (m, 2H), 7.60 (m, 4H), 7.70 (m, 2H), 8.55 (s, 2H).

### EXAMPLE 91

### General Procedure B

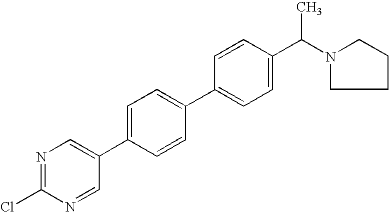

### 2-Chloro-5-[4′-(1-pyrrolidin-1-ylethyl)-biphenyl-4-yl]-pyrimidine

Prepared according to the procedure of Example 70, starting with 71 mg (0.19 mmol) of (±)1-{1-[4′-(4,4,5,5-tetramethyl-[1,3,2]dioxaborolan-2-yl)-biphenyl-4-yl]-ethyl}-pyrrolidine (Intermediate 3) and 55 mg (0.28 mmol) of 5-bromo-2-chloropyrimidine

Mass spectrum (m/z) calcd for C₂₂H₂₂ClN₃: 363.89; obsd: 364 (M+1), 366.

### EXAMPLE 92

### General Procedure B

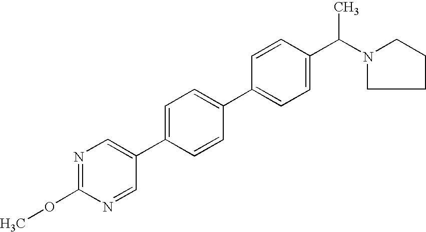

### 2-Methoxy-5-[4′-(1-pyrrolidin-1-ylethyl)-biphenyl-4-yl]-pyrimidine

Prepared according to the procedure described in example 70, starting with 165 mg (0.5 mmol) of 1-[1-(4′-bromobiphenyl-4-yl)-ethyl]-pyrrolidine and 115 mg (0.75 mmol) of 2-methoxypyrimidine-5-boronic acid. Conversion of the free base to the hydrochloride salt as described precviously gave 267 mg of a white solid.

Mass spectrum (m/z) calcd for C₂₃H₂₅N₃O: 359.47; obsd: 360 (M+1).

1H-nmr (CDCl₃, 400 MHz, free base): δ 1.42 (d, 3H), 1.76 (m, 4H), 2.41 (m, 2H), 2.55 (m, 2H), 3.22 (m, 1H), 4.05 (s, 3H), 7.41 (m, 2H), 7.56 (m, 4H), 7.68 (m, 2H), 8.75 (s, 2H).

### EXAMPLE 93

### General Procedure B

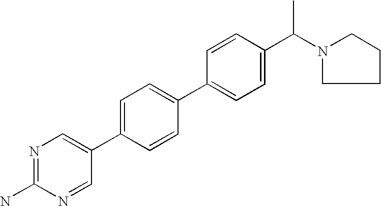

### 5-[4′-(1-Pyrrolidin-1-ylethyl)-biphenyl-4-yl]-pyrimidin-2-ylamine

Prepared according to the procedure described in example 70, starting with 190 mg (0.5 mmol) of (±)1-[1-[4′-(4,4,5,5-tetramethyl-[1,3,2]dioxaborolan-2-yl)-biphenyl-4-yl]-ethyl]-pyrrolidine (Intermediate 3) and 130 mg (0.75 mmol) of 2-amino-5-bromopyrimidine. Conversion of the free base to the hydrochloride salt as described precviously gave 23 mg of a pale yellow solid.

Mass spectrum (m/z) calcd for C₂₂H₂₄N₄: 344.46; obsd: 345 (M+1).

1H-nmr (CDCl₃, 400 MHz, free base): δ 1.44 (d, 3H), 1.78 (m, 4H), 2.43 (m, 2H), 2.59 (m, 2H), 3.25 (m, 1H), 5.10 (s, 2H), 7.42 (m, 2H), 7.55 (m, 4H), 7.66 (m, 2H), 8.57 (m, 2H).

### EXAMPLE 94

### General Procedure B

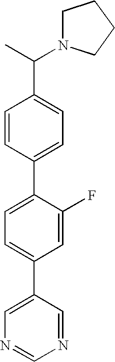

### 5-[2-Fluoro-4′-(1-pyrrolidin-1-ylethyl)-biphenyl-4-yl]-pyrimidine

Prepared according to the method in Example 70 starting with 120 mg (0.41 mmol) of 1-[1-(4′-bromo-2′-fluorobiphenyl-4-yl)-ethyl]-pyrrolidine (intermediate 7) and 51 mg (0.41 mmol) of pyrimidine-5-boronic acid. The HCl salt was prepared as described previously and isolated as an off-white solid, 39 mg.

M.P. 179.9-181.3° C.

Mass spectrum (m/z) calcd for C₂₂H₂₂FN₃: 347.43; obsd: 348 (M+1).

1H-nmr (DMSO-d₆, 400 MHz, HCl salt): δ 1.64 (d, 3H), 1.90 (m, 4H), 2.85 (m, 1H), 2.93 (bs, 1H), 3.10 (m, 1H), 3.66 (m, 1H), 4.43 (m, 1H), 7.67 (m, 3H), 7.77 (m, 3H), 7.88 (dd, 1H), 9.20 (s, 1H), 9.22 (s, 2H), 11.5 (bs, 1H).

### EXAMPLE 95

### General Procedure B

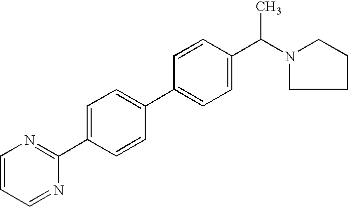

### 2-[4′-(1-Pyrrolidin-1-ylethyl)-biphenyl-4-yl]-pyrimidine

Prepared according to the procedure described in example 70, starting with 86 mg (0.23 mmol) of (±)1-{1-[4′-(4,4,5,5-tetramethyl-[1,3,2]dioxaborolan-2-yl)-biphenyl-4-yl]-ethyl}-pyrrolidine (Intermediate 3) and 54 mg (0.34 mmol) of 2-bromopyrimidine. Conversion of the free base to the hydrochloride salt as described precviously gave 12 mg of a pale yellow solid.

Mass spectrum (m/z) calcd for C₂₂H₂₃N₃: 329.44; obsd: 330 (M+1).

1H-nmr (CDCl₃, 400 MHz, free base): δ 1.43 (d, 3H), 1.60 (bm, 1H), 1.77 (m, 3H), 2.41 (m, 2H), 2.57 (m, 2H), 3.24 (m, 1H), 7.16-7.72 (m, 9H), 8.48 (m, 1H), 8.80 (s, 1H).

### EXAMPLE 96

### General Procedure E

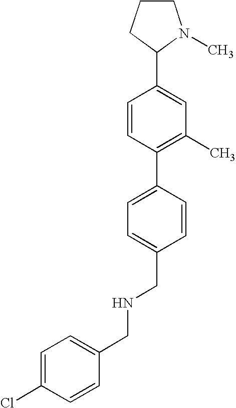

### (4-Chlorobenzyl)-[2′-methyl-4′-(1-methylpyrrolidin-2-yl)-biphenyl-4-ylmethyl]-amine

Prepared using the same procedure described for example 79, starting with 4-chlorobenzylamine (157 mg, 1.11 mmol) and 2′-methyl-4′-(1-methylpyrrolidin-2-yl)-biphenyl-4-carboxaldehyde (155 mg, 0.55 mmol, intermediate 5) in 10 mL ethanol at rt, followed by addition of 315 mg (1.11 mmol) of titanium isopropoxide.

M.p. 238.2-239.3° C.

Mass spectrum (m/z) calcd for C₂₆H₂₉ClN₂: 404.98; obsd: 405 (M+1), 407. 1H-nmr (DMSO-d₆, 400 MHz, dihydrochloride salt): δ 2.09 (bs, 2H), 2.22 (s, 3H), 2.35 (m, 1H), 2.58 (s, 3H), 3.30 (bs, 1H), 3.29 (bs, 1H), 3.70 (bs, 1H), 4.16 (bs, 3H), 4.30 (bs, 1H), 7.24 (d, 1H), 7.34 (dd, 2H), 7.47 (dd, 2H), 7.59 (m, 6H).

### EXAMPLE 97

### General Procedure E

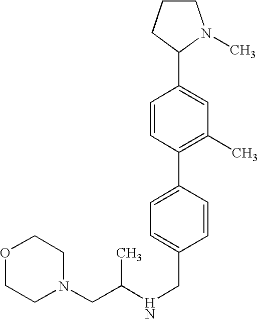

### [2′-Methyl-4′-(1-methylpyrrolidin-2-yl)-biphenyl-4-ylmethyl]-(1-methyl-2-morpholin-4-ylethyl)-amine

Prepared as in Example 96 above, starting with 2′-methyl-4′-(1-methylpyrrolidin-2-yl)-biphenyl-4-carboxaldehyde (304 mg, 1.09 mmol, intermediate 5) and N-(2-aminopropyl)-morpholine (313 mg, 2.18 mmol) in 10 mL ethanol, treated with titanium isopropoxide (619 mg, 2.18 mmol).

Mass spectrum (m/z) calcd for C₂₆H₃₇N₃O: 407.60; obsd: 408 (M+1).

1H-nmr (CDCl₃, 400 MHz, free base): δ 1.80 (m, 2H), 1.97 (m, 1H), 2.10 (m, 2H), 2.20 (s, 3H), 2.23 (s, 3H), 2.32 (m, 4H), 2.77 (m, 1H), 3.02 (t, 1H), 3.25 (t, 1H), 3.62 (bm, 3H), 3.67 (d, 1H), 3.96 (d, 1H), 7.14 (bm, 2H), 7.22 (bs, 1H), 7.28 (q, 4H).

### Determination of Biological Activity

The in vitro affinity of the compounds in the present invention at the rat or human histamine H3 receptors can be determined according to the following procedure. Frozen rat frontal brain or frozen human post-mortem frontal brain is homogenized in 20 volumes of cold 50 mM Tris HCl containing 2 mM MgCl₂ (pH to 7.4 at 4 degrees C.). The homogenate is then centrifuged at 45,000 G for 10 minutes. The supernatant is decanted and the membrane pellet re-suspended by Polytron in cold 50 mM Tris HCl containing 2 mM MgCl₂ (pH to 7.4 at 4 degrees C.) and centrifuged again. The final pellet is re-suspended in 50 mM Tris HCl containing 2 mM MgCl₂ (pH to 7.4 at 25 degrees C.) at a concentration of 12 mg/mL. Dilutions of compounds are made in 10% DMSO/50 mM Tris buffer (pH 7.4) (at 10×final concentration, so that the final DMSO concentration is 1%). Incubations are initiated by the addition of membranes (200 microliters) to 96 well V-bottom polypropylene plates containing 25 microliters of drug dilutions and 25 microliters of radioligand (1 nM final concentration ³H—N-methylhistamine). After a 1 hour incubation, assay samples are rapidly filtered through Whatman GF/B filters and rinsed with ice-cold 50 mM Tris buffer (pH 7.4) using a Skatron cell harvester. Radioactivity is quantified using a BetaPlate scintillation counter. The percent inhibition of specific binding can then be determined for each dose of the compound, and an IC50 or Ki value can be calculated from these results.

TABLE 1
Rat H3 Binding for selected compounds

| Example # | Rat H3 activity (Kᵢ, nM) |
|---|---|
| 1 | 39.4 |
| 2 | 137 |
| 4 | 35.8 |
| 5 | 45 |
| 6 | 61.8 |
| 7 | 22.7 |
| 9 | 50 |
| 20 | 25.2 |
| 24 | 52 |
| 28 | 13.6 |
| 35 | 22.7 |
| 39 | >140 |
| 72 | 3.5 |
| 73 | 72.6 |
| 74 | 10 |
| 75 | 3.5 |
| 76 | 10.8 |
| 77 | 24.3 |
| 78 | 21.3 |
| 79 | 13.9 |
| 90 | 44 |
| 91 | 38 |
| 92 | 42 |
| 93 | 105 |
| 94 | 23 |
| 96 | 14.7 |

1. A compound of formula I 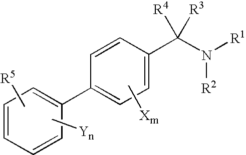
or a pharmaceutically acceptable salt thereof, wherein:
m=1, 2 or 3
n=1, 2, or 3
X and Y are independently selected from H, F, Cl, Br, 1, C₁-C₆ alkyl (optionally substituted by F), C₁-C₆ alkoxyl (optionally substituted by F), (C₁-C₆ alkyl)-S(O)ₚ (optionally substituted by F, NO₂, COOH, COOR⁹, CONR¹⁰R¹¹;
wherein R⁹ is hydrogen, C₁-C₆ alkyl (optionally substituted by F), aryl, heteroaryl, C₁-C₆ alkyl-aryl, C₁-C₆alkyl-heteroaryl;
R¹⁰ and R¹¹ are chosen from the group consisting of hydrogen, C₁-C₆ alkyl, aryl, heteroaryl, C₁-C₆ alkyl-(aryl), or R¹⁰ and R¹¹ taken together with the nitrogen to which they are attached form a ring of 4-8 atoms with up to 3 additional heteroatoms including N, O, S; and
p=0, 1 or 2.
R¹ and R² are independently selected from the group consisting of hydrogen;
C₁-C₈ alkyl optionally substituted with 1 to 4 halogens or OH;
C₃-C₇ cycloalkyl;
C₆-C₁₄ aryl;
3-8-membered heterocycloalkyl optionally substituted with a C₁-C₄ alkyl-carbonyl group;
C₆-C₁₀arylsulfonyl optionally substituted with C₁-C₂ alkyl; and
5-10-membered heteroaryl;
R³ is selected from the group consisting of C₁-C₈ alkyl optionally substituted with 1 to 4 halogens;
C₃-C₇ cycloalkyl;
C₆-C₁₄ aryl; or
R¹ and R² together with the nitrogen of the NR¹R² group form a 4-7 member ring, wherein one of the carbons in the ring is optionally replaced by O, S, NR⁶, or CO, and the ring is optionally fused to a C₆-C₁₀ arylene and is optionally substituted at a ring carbon with one or two C₁-C₄ alkyl groups, wherein R⁶ is
hydrogen;
C₁-C₈ alkyl optionally substituted with 1 to 4 halogens;
5-10-membered heteroaryl optionally substituted with a substituent selected from the group consisting of halogen, C₁-C₄ alkyl, C₁-C₂ alkoxy, C₆-C₁₀ aryl, C₁-C₄ alkylaminocarbonyl, cyano;
C₆-C₁₀ aryl optionally substituted with one or two C₁-C₂ alkyl; or
C₁-C₄ alkyl-carbonyl; or
R¹ and R³ together with the nitrogen of the NR¹R³ group form a 4-7 member ring, wherein one of the carbons in the ring is optionally replaced by O, S, NR^{6′}, or CO, and the ring is optionally fused to a C₆-C₁₀ arylene and is optionally substituted at a ring carbon with one or two C₁-C₄ alkyl groups, wherein R^{6′} is
hydrogen;
C₁-C₈ alkyl optionally substituted with 1 to 4 halogens;
5-10-membered heteroaryl optionally substituted with a substituent selected from the group consisting of halogen, C₁-C₄ alkyl, C₁-C₂ alkoxy, C₆-C₁₀ aryl, C₁-C₄ alkylaminocarbonyl, cyano;
C₆-C₁₀ aryl optionally substituted with one or two C₁-C₂ alkyl; or
C₁-C₄ alkyl-carbonyl;
R⁴ is
hydrogen, or
C₁-C₈ alkyl optionally substituted with 1 to 4 halogens;
R⁵ is (CH)ₜ—W, wherein W is a 5-7 member heteroaryl or heterocycloalkyl ring, optionally substituted by one or more substituents R⁷ and optionally fused to an aryl, 5-10-membered heteroaryl or C₄-C₈ cycloalkyl ring and wherein R⁷ is selected from the group consisting of
hydrogen;
F, Cl, Br or I;
C₁-C₆ alkyl optionally substituted by F;
C₁-C₆ alkoxyl optionally substituted by F;
(C₁-C₆ alkyl)-S(O)ₚ (optionally substituted by F;
NO₂; NH2, NHR1′, NR1′R2′, wherein R^{1′} and R^{2′} are independently as defined in R¹ and R² above;
COOH, COOR^{9′}, CONR^{10′}R^{11′}, wherein R^{9′}, R^{10′} and R^{11′} are as independently defined in R⁹, R¹⁰ and R¹¹ above, and
t is 0, 1 or 2.

2. The compound of claim 1, wherein R¹ is methyl, R² is methyl, and R³ is methyl.

3. The compound of claim 1, wherein R¹ and R³ together with the nitrogen to which they are attached form the 5-membered pyrrolidine ring and R² is methyl.

4. The compound of claim 1, wherein R⁷ is hydrogen or C₁-C₆ alkyl.

5. The compound of claim 1, wherein (B) R¹ and R² together with the nitrogen to which they are attached form the 5-membered pyrrolidine ring, and R³ is methyl.

6. The compound of claim 1, wherein (D) R¹ and R² together with the nitrogen to which they are attached form the 6-membered piperidine ring, and R³ is methyl.

7. The compound of claim 1, wherein (E) R¹ and R³ together with the nitrogen to which they are attached form the 6-membered piperidine ring, and R is methyl.

8. The compound of claim 1, wherein the halogen in C₁-C₈ of R¹, R² and R³ is Fluorine.

9. The compounds of formula I in of claim 1 wherein the compound is selected from the group consisting of:
(R)-3-[4′-(1-Pyrrolidin-1-ylethyl)-biphenyl-4-yl]-piperidine,
(S)-5-[4′-(1-Pyrrolidin-1-ylethyl)-biphenyl-4-yl]-pyrimidine,
(R)-4-[4′-(1-Pyrrolidin-1-ylethyl)-biphenyl-4-yl]-piperidine,
(S)-4-[4′-(1-Pyrrolidin-1-ylethyl)-biphenyl-4-yl]-piperidine,
(±)-4-[4′-(1-Pyrrolidin-1-ylethyl)-biphenyl-4-yl]-piperidine,
(±)-3-[4′-(1-Pyrrolidin-1-ylethyl)-biphenyl-4-yl]-pyridine,
(R)-2-[4′-(1-Pyrrolidin-1-ylethyl)-biphenyl-4-yl]-piperidine,
(±)-Dimethyl-[1-(4′-pyridin-4-yl-biphenyl-4-yl)-ethyl]-amine,
(R)-5-[4′-(1-Pyrrolidin-1-ylethyl)-biphenyl-4-yl]-pyrimidine,
(S)-5-[4′-(1-Pyrrolidin-1-ylethyl)-biphenyl-4-yl]-pyrimidine,
(R)-4-[4′-(1-Pyrrolidin-1-ylethyl)-biphenyl-4-yl]-pyridine,
(R)-3-[4′-(1-Pyrrolidin-1-ylethyl)-biphenyl-4-yl]-pyridine,
(±)-3-[4′-(1-Pyrrolidin-1-ylethyl)-biphenyl-4-yl]-pyridine,(±)-1-[1-(4′-Benzo[b]thiophen-2-ylbiphenyl-4-yl)-ethyl]-pyrrolidine,
(±)-4-(1-Pyrrolidin-1-ylethyl)-[1,1′;4′,1″]terphenyl-3″-carbonitrile,
(±)-3,5-Dimethyl-4-[4′-(1-pyrrolidin-1-ylethyl)-biphenyl-4-yl]-isoxazole,
(±)-4″-(1-Pyrrolidin-1-ylethyl)-[1,1′;4′,1″]terphenyl-3-carboxylic acid dimethylamide,
(±)-1-{1-[4′-(2-Phenylcyclopropyl)-biphenyl-4-yl]-ethyl}-pyrrolidine,
(±)-3-Chloro-4-[4′-(1-pyrrolidin-1-ylethyl)-biphenyl-4-yl]-pyridine,
(±)-1-[1-(3″-Methylsulfanyl-[1,1′;4′,1″]terphenyl-4-yl)-ethyl]-pyrrolidine,
(±)-1-{1-[4′-(2,3-Dihydro-benzo[1,4]dioxin-6-yl)-biphenyl-4-yl]-ethyl}-pyrrolidine,
(±)-4″-(1-Pyrrolidin-1-ylethyl)-[1,1′;4′,1″]terphenyl-3-carboxylic acid amide,
(±)-3-Fluoro-4-[4′-(1-pyrrolidin-1-ylethyl)-biphenyl-4-yl]-pyridine,
(±)-5-[4′-(1-Pyrrolidin-1-ylethyl)-biphenyl-4-yl]-pyrimidine,
(±)-1-Methyl-5-[4′-(1-pyrrolidin-1-ylethyl)-biphenyl-4-yl]-1H-indole,
(±)-1-[1-(4′-Benzo[b]thiophen-3-ylbiphenyl-4-yl)-ethyl]-pyrrolidine,
(±)-4″-(1-Pyrrolidin-1-ylethyl)-[1,1′;4′,1″]terphenyl-2-sulfonic acid tert-butyl-amide,
(S)-4-[4′-(1-Pyrrolidin-1-ylethyl)-biphenyl-4-yl]-pyridine,
(±)-4-[4′-(1-Pyrrolidin-1-ylethyl)-biphenyl-4-yl]-pyridine,
(±)-1-[1-(4′-Furan-2-ylbiphenyl-4-yl)-ethyl]-pyrrolidine,
(±)-1-[1-(4′-Benzo[1,3]dioxol-5-ylbiphenyl-4-yl)-ethyl]-pyrrolidine,
(±)-5-[4′-(1-Pyrrolidin-1-ylethyl)-biphenyl-4-yl]-isoquinoline,
(±)-4″-(1-Pyrrolidin-1-ylethyl)-[1,1′;4′,1″]terphenyl-2-carboxylic acid diisopropylamide,
(±)-[4-(1-Pyrrolidin-1-ylethyl)-[1,1′;4′,1″]terphenyl-4″-yl]-methanol,
(±)-[4-(1-Pyrrolidin-1-ylethyl)-[1,1′;4′,1″]terphenyl-3″-yl]-methanol,
(±)-[4″-(1-Pyrrolidin-1-ylethyl)-[1,1′;4′,1″]terphenyl-2-yl]-methanol,
(±)-1-[4″-(1-Pyrrolidin-1-ylethyl)-[1,1′;4′,1″]terphenyl-3-yl]-1H-pyrazole,
(±)-N-[4″-(1-Pyrrolidin-1-ylethyl)-[1,1′;4′,1″]terphenyl-3-yl]-acetamide,
(±)-4-(1-Pyrrolidin-1-ylethyl)-[1,1′;4′,1″]terphenyl-4″-carbonitrile,
(±)-1-[1-(4-Methanesulfonyl-[1,1′;4′,1″]terphenyl-4″-yl)-ethyl]-pyrrolidine,
(±)-1-[1-(3,5-Dichloro-[1,1′;4′,1″]terphenyl-4″-yl)-ethyl]-pyrrolidine,
(±)-1-[1-(3″,4″-Dichloro-[1,1′;4′,1″]terphenyl-4-yl)-ethyl]-pyrrolidine,
(±)-1-[1-(4′-Thiophen-3-ylbiphenyl-4-yl)-ethyl]-pyrrolidine,
(±)-{1-[4′-(3-Fluoropyridin-4-yl)-biphenyl-4-yl]-ethyl}-dimethylamine,
(±)-{1-[4′-(2,3-Dihydro-benzo[1,4]dioxin-6-yl)-biphenyl-4-yl]-ethyl}-dimethylamine,
(±)-Dimethyl-{1-[4′-(1-methyl-1H-indol-5-yl)-biphenyl-4-yl]-ethyl}-amine,
(±)-[1-(4′-Benzo[b]thiophen-3-ylbiphenyl-4-yl)-ethyl]-dimethylamine,
(±)-4″-(1-Dimethylaminoethyl)-[1,1′;4′,1″]terphenyl-2-sulfonic acid tert-butyl-amide,
(±)-4″-(1-Dimethylaminoethyl)-[1,1′;4′,1″]terphenyl-3-carbonitrile,
(±)-4″-(1-Dimethylaminoethyl)-3-methoxy-[1,1′;4′,1″]terphenyl-2-carboxylic acid diisopropylamide,
(±)-{1-[4′-(3,5-Dimethylisoxazol-4-yl)-biphenyl-4-yl]-ethyl}-dimethylamine,
(±)-4″-(1-Dimethylaminoethyl)-[1,1′;4′,1″]terphenyl-2-carboxylic acid diisopropylamide,
(±)-Dimethyl-[1-(4′-thiophen-2-ylbiphenyl-4-yl)-ethyl]-amine,
(±)-Dimethyl-[1-(4′-thiophen-3-ylbiphenyl-4-yl)-ethyl]-amine,
(±)-[1-(4′-Benzofuran-2-ylbiphenyl-4-yl)-ethyl]-dimethylamine,
(±)-[4-(1-Dimethylaminoethyl)-[1,1′;4′,1″]terphenyl-4″-yl]-methanol,
(±)-[1-(4′-Furan-2-ylbiphenyl-4-yl)-ethyl]-dimethylamine,
(±)-[1-(4′-Benzo[1,3]dioxol-5-ylbiphenyl-4-yl)-ethyl]-dimethylamine,
(±)-[4″-(1-Dimethylaminoethyl)-[1,1′;4′,1″]terphenyl-3-yl]-methanol,
(±)-[4″-(1-Dimethylaminoethyl)-[1,1′;4′,1″]terphenyl-2-yl]-methanol,
(±)-Dimethyl-[1-(4′-pyridin-4-ylbiphenyl-4-yl)-ethyl]-amine,
(±)-[1-(4′-Furan-3-ylbiphenyl-4-yl)-ethyl]-dimethylamine,
(±)-N-[4″-(1-Dimethylaminoethyl)-[1,1′;4′,1″]terphenyl-3-yl]-acetamide,
(±)-Dimethyl-[1-(2-methylsulfanyl-[1,1′;4′,1″]terphenyl-4″-yl)-ethyl]-amine,
(±)-4-(1-Dimethylaminoethyl)-[1,1′;4′,1″]terphenyl-4″-carbonitrile,
(±)-[1-(4-Methanesulfonyl-[1,1′;4′,1″]terphenyl-4″-yl)-ethyl]-dimethylamine,
(±)-[1-(4-Ethanesulfonyl-[1,1′;4′,1″]terphenyl-4′-yl)-ethyl]-dimethylamine,
(±)-[1-(4′-Isoquinolin-5-ylbiphenyl-4-yl)-ethyl]-dimethylamine,
(±)-Dimethyl-[1-(3-pyrazol-1-yl-[1,1′;4′,1″]terphenyl-4″-yl)-ethyl]-amine,
(±)-Dimethyl-[1-(3-methylsulfanyl-[1,1′;4′,1″]terphenyl-4″-yl)-ethyl]-amine,
(±)-{1-[4′-(3-Chloropyridin-4-yl)-biphenyl-4-yl]-ethyl}-dimethylamine,
(±)-Dimethyl-[1-(4′-pyrimidin-5-ylbiphenyl-4-yl)-ethyl]-amine,
(±)-{1-[4′-(2,4-Dimethoxypyrimidin-5-yl)-biphenyl-4-yl]-ethyl}-dimethylamine,
(R)-2-[4′-(1-Pyrrolidin-1-ylethyl)-biphenyl-4-yl]-pyridine,
(±)-2-[4′-(1-Pyrrolidin-1-ylethyl)-biphenyl-4-yl]-piperidine,
(R)-1-Methyl-4-[4′-(1-pyrrolidin-1-ylethyl)-biphenyl-4-yl]-piperidine,
(R)-1-Ethyl-4-[4′-(1-pyrrolidin-1-ylethyl)-biphenyl-4-yl]-piperidine,
(±)-1-[2′-Methyl-4′-(1-methylpyrrolidin-2-yl)-biphenyl-4-ylmethyl]-piperidine,
(R)-2,4-Dimethoxy-5-[4′-(1-pyrrolidin-1-ylethyl)-biphenyl-4-yl]-pyrimidine,
1-Methanesulfonyl-4-[4′-(1-pyrrolidin-1-ylethyl)-biphenyl-4-yl]-piperidine,
5-[3,5-Difluoro-4′-(1-pyrrolidin-1-ylethyl)-biphenyl-4-yl]-pyrimidin-2-ol,
5-[2,5-Difluoro-4′-(1-pyrrolidin-1-ylethyl)-biphenyl-4-yl]-pyrimidin-2-ol,
5-[3-Fluoro-4′-(1-pyrrolidin-1-ylethyl)-biphenyl-4-yl]-pyrimidin-2-ol,
5-[3,5-Difluoro-4′-(1-pyrrolidin-1-ylethyl)-biphenyl-4-yl]-pyrimidine,
5-[3,5-Difluoro-4′-(1-pyrrolidin-1-ylethyl)-biphenyl-4-yl]-2-methoxypyrimidine,
5-[2,5-Difluoro-4′-(1-pyrrolidin-1-ylethyl)-biphenyl-4-yl]-pyrimidine,
5-[2,5-Difluoro-4′-(1-pyrrolidin-1-ylethyl)-biphenyl-4-yl]-2-methoxy-pyrimidine,
5-[3-Fluoro-4′-(1-pyrrolidin-1-ylethyl)-biphenyl-4-yl]-pyrimidine,
5-[3-Fluoro-4′-(1-pyrrolidin-1-yl ethyl)-biphenyl-4-yl]-2-methoxypyrimidine,
5-[4′-(1-Pyrrolidin-1-ylethyl)-biphenyl-4-yl]-pyrimidin-2-ol,
2-Chloro-5-[4′-(1-pyrrolidin-1-ylethyl)-biphenyl-4-yl]-pyrimidine,
2-Methoxy-5-[4′-(1-pyrrolidin-1-ylethyl)-biphenyl-4-yl]-pyrimidine,
5-[4′-(1-Pyrrolidin-1-ylethyl)-biphenyl-4-yl]-pyrimidin-2-ylamine,
5-[2-Fluoro-4′-(1-pyrrolidin-1-ylethyl)-biphenyl-4-yl]-pyrimidine,
2-[4′-(1-Pyrrolidin-1-ylethyl)-biphenyl-4-yl]-pyrimidine,
(4-Chlorobenzyl)-[2′-methyl-4′-(1-methylpyrrolidin-2-yl)-biphenyl-4-ylmethyl]-amine, and
[2′-Methyl-4′-(1-methylpyrrolidin-2-yl)-biphenyl-4-ylmethyl]-(1-methyl-2-morpholin-4-ylethyl)-amine.

10. The compound of formula of claim 1, wherein the compound is selected from the group consisting of:
1-[4′-(1-Pyrrolidin-1-ylethyl)-biphenyl-4-yl]-1H-pyrazole,
2-[4′-(1-Pyrrolidin-1-ylethyl)-biphenyl-4-yl]-pyrazine,
1-[4′-(1-Pyrrolidin-1-ylethyl)-biphenyl-4-yl]-1H-[1,2,4]triazole,
4-[4′-(1-Pyrrolidin-1-ylethyl)-biphenyl-4-yl]-4H-[1,2,4]triazole,
2,4-Dimethyl-1-[4′-(1-pyrrolidin-1-ylethyl)-biphenyl-4-yl]-1H-imidazole,
2-Methyl-5-[4′-(1-pyrrolidin-1-ylethyl)-biphenyl-4-yl]-pyrimidine,
2-Fluoro-5-[4′-(1-pyrrolidin-1-ylethyl)-biphenyl-4-yl]-pyrimidine,
2-Fluoro-4-methyl-5-[4′-(1-pyrrolidin-1-ylethyl)-biphenyl-4-yl]-pyrimidine,
5-[3-Methyl-4′-(1-pyrrolidin-1-ylethyl)-biphenyl-4-yl]-pyrimidine,
5-[3,5-Dimethyl-4′-(1-pyrrolidin-1-ylethyl)-biphenyl-4-yl]-pyrimidine,
2,6-Dimethyl-3-[4′-(1-pyrrolidin-1-ylethyl)-biphenyl-4-yl]-pyridine,
2-Methyl-5-[4′-(1-pyrrolidin-1-ylethyl)-biphenyl-4-yl]-pyridine,
5-{4′-[1-(2-Methylpyrrolidin-1-yl)-ethyl]-biphenyl-4-yl}-pyrimidine,
5-{4′-[1-(2,5-Dimethylpyrrolidin-1-yl)-ethyl]-biphenyl-4-yl}-pyrimidine,
5-{4′-[1-(2,2-Dimethylpyrrolidin-1-yl)-ethyl]-biphenyl-4-yl}-pyrimidine,
5-{4′-[1-(3,3-Dimethylpyrrolidin-1-yl)-ethyl]-biphenyl-4-yl}-pyrimidine,
5-[4′-(1-Piperidin-1-ylethyl)-biphenyl-4-yl]-pyrimidine,
4-[1-(4′-Pyrimidin-5-yl-biphenyl-4-yl)-ethyl]-morpholine,
5-[4′-(1-Methylpiperidin-2-yl)-biphenyl-4-yl]-pyrimidine,
4-Methyl-3-(4′-pyrimidin-5-yl-biphenyl-4-yl)-morpholine,
5-[4′-(1,4-Dimethylpiperazin-2-yl)-biphenyl-4-yl]-pyrimidine,
5-[4′-(1,5-Dimethylpyrrolidin-2-yl)-biphenyl-4-yl]-pyrimidine,
3-(4′-Pyrimidin-5-yl-biphenyl-4-yl)-octahydro-indolizine,
5-[4′-(1-Isopropyl-pyrrolidin-2-yl)-biphenyl-4-yl]-pyrimidine,
5-[4′-(1-Benzylpyrrolidin-2-yl)-biphenyl-4-yl]-pyrimidine,
5-[2′-Fluoro-4′-(1-methylpyrrolidin-2-yl)-biphenyl-4-yl]-pyrimidine,
5-[2′,6′-Difluoro-4′-(1-methylpyrrolidin-2-yl)-biphenyl-4-yl]-pyrimidine,
5-[2-Methyl-4′-(1-methylpyrrolidin-2-yl)-biphenyl-4-yl]-pyrimidine,
5-[4′-(1-Methyl-1-pyrrolidin-1-yl-ethyl)-biphenyl-4-yl]-pyrimidine,
2-Methyl-4-[4′-(1-pyrrolidin-1-yl-ethyl)-biphenyl-4-yl]-piperidine,
2,6-Dimethyl-4-[4′-(1-pyrrolidin-1-yl-ethyl)-biphenyl-4-yl]-piperidine,
1,2,6-Trimethyl-4-[4′-(1-pyrrolidin-1-yl-ethyl)-biphenyl-4-yl]-piperidine,
2-Methyl-6-[4′-(1-pyrrolidin-1-yl-ethyl)-biphenyl-4-yl]-piperidine,
3,6-Dimethyl-2-[4′-(1-pyrrolidin-1-yl-ethyl)-biphenyl-4-yl]-piperidine,
1,2-Dimethyl-6-[4′-(1-pyrrolidin-1-yl-ethyl)-biphenyl-4-yl]-piperidine,
1-[4′-(1-Methylpyrrolidin-2-yl)-biphenyl-4-ylmethyl]-pyrrolidine,
1-[4′-(1-Methylpyrrolidin-2-yl)-biphenyl-4-ylmethyl]-2methylpyrrolidine,
2-[4′-(1-Methylpyrrolidin-2-yl)-biphenyl-4-ylmethyl]-2,3-dihydro-1H-isoindole,
2-[4′-(1-Methylpyrrolidin-2-yl)-biphenyl-4-ylmethyl]-octahydro-isoindole,
1-[4′-(1-Methylpyrrolidin-2-yl)-biphenyl-4-ylmethyl]-1-azaspiro[4.5]decane,
8-[4′-(1-Methylpyrrolidin-2-yl)-biphenyl-4-ylmethyl]-8-azabicyclo[3.2.1]octane,
2-[4′-(1-Methylpyrrolidin-2-yl)-biphenyl-4-ylmethyl]-2-azabicyclo[2.2.2]octane,
4-[4′-(1-Methylpyrrolidin-2-yl)-biphenyl-4-ylmethyl]-morpholine,
4-[4′-(1-Methylpyrrolidin-2-yl)-biphenyl-4-ylmethyl]-thiomorpholine,
4-[4′-(1-Methylpyrrolidin-2-yl)-biphenyl-4-ylmethyl]-thiomorpholine 1-oxide,
4-[4′-(1-Methylpyrrolidin-2-yl)-biphenyl-4-ylmethyl]-thiomorpholine 1,1-dioxide,
1-[4′-(1-Methylpyrrolidin-2-yl)-biphenyl-4-ylmethyl]-azepine,
Dicyclopropyl-[4′-(1-methylpyrrolidin-2-yl)-biphenyl-4-ylmethyl]-amine,
Methyl-[4′-(1-methylpyrrolidin-2-yl)-biphenyl-4-ylmethyl]-phenylamine,
1-[4′-(1-Methylpyrrolidin-2-yl)-biphenyl-4-ylmethyl]-2,3-dihydro-1H-indole,
3-[4′-(1-Methylpyrrolidin-2-yl)-biphenyl-4-ylmethyl]-2,3-dihydro-benzothiazole,
Cyclohexyl-methyl-[4′-(1-methyl-pyrrolidin-2-yl)-biphenyl-4-ylmethyl]-amine,
Methyl-[4′-(1-methylpyrrolidin-2-yl)-biphenyl-4-ylmethyl]-(tetrahydropyran-4-yl)-amine,
4-[4′-(1-Pyrrolidin-1-ylpropyl)-biphenyl-4-yl]-pyridine,
2-Methyl-5-[1-(4′-pyridin-4-ylbiphenyl-4-yl)-ethyl]-octahydro-pyrrolo[3,4-c]pyrrole,
2-[1-(4′-Pyridin-4-ylbiphenyl-4-yl)-ethyl]-octahydro-isoindole,
(1-Azabicyclo[2.2.2]oct-3-yl)-[1-(4′-pyridin-4-ylbiphenyl-4-yl)-ethyl]-amine,
Dimethyl-[phenyl-(4′-pyridin-4-ylbiphenyl-4-yl)-methyl]-amine,
tert-Butyl-[1-(4′-pyridin-4-yl biphenyl-4-yl)-ethyl]-amine,
tert-Butyl-methyl-[1-(4′-pyridin-4-ylbiphenyl-4-yl)-ethyl]-amine,
4-[4′-(1-Pyrrolidin-1-ylethyl)-biphenyl-4-yl]-4H-[1,2,4]triazole, and
1-[4′-(1-Pyrrolidin-1-ylethyl)-biphenyl-4-yl]-1H-imidazole.

11. A pharmaceutical composition for treating a disorder or condition that may be treated by antagonizing histamine-3 receptors, the composition comprising a compound of formula I as described in claim 1, and optionally a pharmaceutically acceptable carrier.

12. A method of treatment of a disorder or condition that may be treated by antagonizing histamine-3 receptors, the method comprising administering to a mammal in need of such treatment a compound of formula I as described in claim 1.

13. A pharmaceutical composition comprising a compound of formula I as described in claim 1, and optionally a pharmaceutically acceptable carrier.

14. A method of treatment of a disorder or condition selected from the group consisting of depression, mood disorders, schizophrenia, anxiety disorders, Alzheimer's disease, attention-deficit hyperactivity disorder (ADHD), psychotic disorders, sleep disorders, obesity, dizziness, epilepsy, motion sickness, respiratory diseases, allergy, allergy-induced airway responses, allergic rhinitis, nasal congestion, allergic congestion, congestion, hypotension, cardiovascular disease, diseases of the GI tract, hyper and hypo motility and acidic secretion of the gastro-intestinal tract, the method comprising administering to a mammal in need of such treatment a compound of formula I as described in claim 1.

15. The method of claim 14, wherein the disorder or condition is selected from the group consisting of anxiety disorders, attention-deficit hyperactivity disorder, respiratory diseases, and obesity.

16. The method of claim 14, wherein the disorder or condition is a respiratory disease selected from the group consisting of adult respiratory distress syndrome, acute respiratory distress syndrome, bronchitis, chronic bronchitis, chronic obstructive pulmonary disease, cystic fibrosis, asthma, emphysema, rhinitis and chronic sinusitis.

17. A pharmaceutical composition for treating allergic rhinitis, nasal congestion or allergic congestion comprising
a) an H3 receptor antagonist compound of formula 1; or a pharmaceutically acceptable salt thereof;
b) an H1 receptor antagonist or a pharmaceutically acceptable salt thereof; and
c) a pharmaceutically acceptable carrier;
wherein the active ingredients (a) and (b) above are present in amounts that render the composition effective in treating allergy rhinitis, nasal congestion or allergic congestion

18. A pharmaceutical composition for treating depression and mood disorder comprising:
a) an H3 receptor antagonist compound of Formula 1 or a pharmaceutically acceptable salt thereof;
b) a neurotransmitter re-uptake blocker or a pharmaceutically acceptable salt thereof;
c) a pharmaceutically acceptable carrier;
wherein the active ingredients (a) and (b) above are present in amounts that render the composition effective in treating depression and mood disorder.

19. The composition according to claim 18 wherein the H3 receptor antagonist and the neurotransmitter blocker are given simultaneously.

20. The composition according to claim 17 wherein the H3 receptor antagonist and the H1 receptor antagonist are given simultaneously.

21. The pharmaceutical composition of claim 18 wherein the neurotransmitter uptake blocker are selected the group consisting of sertraline, fluoxetine and paroxetine.

22. The pharmaceutical composition of claim 17, wherein the H1 receptor antagonist is cetirizine.
